(19) United States
(12) Patent Application Publication
(10) Pub. No.: US 2014/0147860 A1
(43) Pub. Date: May 29, 2014

(54) Acoustic Cytometry Methods and Protocols

(71) Applicants: Gregory Kaduchak, Eugene, OR (US); Michael Ward, Eugene, OR (US); Jolene Bradford, Eugene, OR (US); Bradley Dubbels, Eugene, OR (US); Barbara Seredick, Eugene, OR (US); Rickie Kerndt, Eugene, OR (US); Kristi Haataja, Tracy, CA (US); April Anderson, Eugene, OR (US); Penny Melquist, Eugene, OR (US); Christopher Langsdorf, Eugene, OR (US); Justin Hicks, Eugene, OR (US); Kathleen Kihn, Lorane, OR (US)

(72) Inventors: Gregory Kaduchak, Eugene, OR (US); Michael Ward, Eugene, OR (US); Jolene Bradford, Eugene, OR (US); Bradley Dubbels, Eugene, OR (US); Barbara Seredick, Eugene, OR (US); Rickie Kerndt, Eugene, OR (US); Kristi Haataja, Tracy, CA (US); April Anderson, Eugene, OR (US); Penny Melquist, Eugene, OR (US); Christopher Langsdorf, Eugene, OR (US); Justin Hicks, Eugene, OR (US); Kathleen Kihn, Lorane, OR (US)

(73) Assignee: LIFE TECHNOLOGIES CORPORATION, Carlsbad, CA (US)

(21) Appl. No.: 14/129,777

(22) PCT Filed: Jun. 27, 2012
(86) PCT No.: PCT/US2012/044463

§ 371 Date: Feb. 14, 2014

Related U.S. Application Data
(60) Provisional application No. 61/501,617, filed on Jun. 27, 2011.
(60) Provisional application No. 61/507,975, filed on Jul. 14, 2011.

(51) Int. Cl. G01N 33/569

(52) U.S. Cl. CPC G01N 33/56966
USPC 435/7.21

(57) ABSTRACT

Various embodiments disclosed herein comprise acoustic cytometry based methods, kits, computer software methods and systems to analyze a variety of bioparticles. In one embodiment, a method for analyzing bioparticles comprises: acoustically focusing one or more bioparticles through an interrogation zone; optically exciting the one or more bioparticles in the interrogation zone with an excitation source; detecting an optical signal from the bioparticles; and analyzing the optical signal to characterize at least one quality or quantity parameter of the bioparticles. Properties of biomolecules that may be analyzed include but are not limited to cell proliferation analysis, live/dead cell discrimination, cell cycle analysis, basic phenotyping, immunophenotyping, rare-event detection, apoptosis, phagocytosis, pinocytosis, detection of phosphoproteins, detection of one or more cellular markers, detection of one or more intracellular marker, detection of cancer cells, detection of pathological markers on a cell, microbial cell analysis and/or picophytoplankton analysis.

### CROSS-REFERENCE TO RELATED APPLICATIONS

This application claims priority of U.S. Provisional Patent Application Ser. No. 61/501,617, entitled “Acoustic Cytometry Methods and Protocols”, filed Jun. 27, 2011, and of U.S. Provisional Patent Application Ser. No. 61/507,975, entitled “Acoustic Cytometry Methods and Protocols”, filed Jul. 14, 2011, and the entire contents of which are incorporated herein by reference.

### FIELD OF THE DISCLOSURE

Embodiments of the present disclosure relate to methods of analyzing bioparticles using acoustic flow cytometry and to kits based on method protocols.

### BACKGROUND

Note that the following discussion refers to a number of publications by author(s) and year of publication, and that due to recent publication dates certain publications are not to be considered as prior art vis-a-vis the present disclosure. Discussion of such publications herein is given for more complete background and is not to be construed as an admission that such publications are prior art for patentability determination purposes.

Flow cytometry is a powerful tool used for analysis of particles and cells in a myriad of applications primarily in bioscience research and medicine. The analytical strength of the technique lies in its ability to parade single particles (including bioparticles such as cells, bacteria and viruses) through the focused spot of light sources, typically a laser or lasers, in rapid succession, at rates up to thousands of particles per second. The high photon flux at this focal spot produces scatter of light by a particle and or emission of light from the particle or labels attached to the particle that can be collected and analyzed. This gives the user a wealth of information about individual particles that can be quickly parleyed into statistical information about populations of particles or cells.

In traditional flow cytometry, particles are flowed through the focused interrogation point where a laser directs a laser beam to a focused point that includes the core diameter within the channel. The sample fluid containing particles is focused to a very small core diameter of around 10-50 microns by flowing sheath fluid around the sample stream at a very high volumetric rate on the order of 100-1000 times the volumetric rate of the sample. This results in very fast linear velocities for the focused particles on the order of meters per second. This in turn means that each particle spends a very limited time in the excitation spot, often only 1-10 microseconds. Further, once the particle passes the interrogation point the particle cannot be redirected to the interrogation point again because the linear flow velocity cannot be reversed. Further, a particle cannot be held at the interrogation point for a user defined period of time for further interrogation because focusing is lost without the flow of the hydrodynamic sheath fluid. Because of the very high photon flux at the excitation point, flow cytometry is still a very sensitive technique, but this fast transit time limits the sensitivity and resolution that can be achieved. Often, greater laser power is used to increase the photon flux in an effort to extract more signal but this approach is limiting in that too much light can often photobleach (or excite to non-radiative states) the fluorophores being used to generate the signal and can increase background Rayleigh scatter, Raman scatter and fluorescence.

Acoustic cytometers, using relatively large dimension flow channels, concentrate particles from the entire volume of the channel to a small acoustic trap in the center of the channel and can therefore offer both controllable flow and high particle analysis rates without resorting to highly concentrated samples.

### SUMMARY

Various embodiments of methods for analyzing bioparticles using acoustic flow cytometry are disclosed. In some embodiments, bioparticles can be intrinsically or naturally fluorescent. In some embodiments, bioparticles may naturally comprise one or more components which when excited with an excitation source during acoustic flow cytometry methods can emit detectable optical signals. In some embodiments a bioparticle to be analyzed may be labeled prior to performing acoustic flow cytometry such that the labeled bioparticle, when excited with an excitation source during acoustic flow cytometry, can emit detectable optical signals.

In one embodiment, the disclosure describes methods for analyzing a bioparticle comprising acoustically focusing one or more bioparticles through an interrogation zone; optically exciting the one or more bioparticles in the interrogation zone with an excitation source; detecting an optical signal from the bioparticles; and analyzing the optical signal to characterize at least one quality or quantity parameter of the bioparticles.

In one embodiment, the present disclosure describes a method for analyzing a labeled bioparticle comprises acoustically focusing one or more labeled bioparticles through an interrogation zone; optically exciting the one or more labeled bioparticles in the interrogation zone with an excitation source; detecting an optical signal from the labeled bioparticles; and analyzing the optical signal to characterize at least one quality or quantity parameter of the labeled bioparticles.

The present methods may be used to analyze a variety of bioparticles including but not limited to a cell, a protein, a peptide, a fusion protein, a tagged protein, a nucleic acid, a DNA molecule, an RNA molecule, a hybrid nucleic acid, a polynucleotide, an oligonucleotide, a triple helical molecule.

In some embodiments, bioparticles may be obtained from a sample which may be a biological sample, a clinical sample, a veterinary sample, a food sample, a beverage sample and/or an environmental sample. Biological samples may include without limitation examples such as cells, cell culture medium, serum, blood, bone marrow, semen, vaginal fluid, urine, spinal fluid, saliva, sputum, bile, peritoneal fluid, amniotic fluid, cerebrospinal fluid, and aspirate from hollow organs, cysts and tissue.

Various types of analysis can be performed on a bioparticle using the present methods such as but not limited to cell proliferation analysis, live/dead cell discrimination, cell cycle analysis, basic phenotyping, immunophenotyping, rare-event detection, apoptosis, phagocytosis, pinocytosis, detection of phosphoproteins, detection of one or more cellular markers, detection of one or more intracellular marker, detection of cancer cells, detection of pathological markers on a cell, microbial cell analysis and/or picophytoplankton analysis.

In some embodiments, a method for analyzing a labeled bioparticle may comprise analyzing a cell (cellular bioparticle) for cell proliferation analysis and may comprise subjecting the labeled cellular bioparticles to a cell proliferation stimulus prior to the acoustic focusing step; followed by acoustically focusing the labeled cellular bioparticles through an interrogation zone; optically exciting the cellular labeled bioparticles in the interrogation zone with an excitation source; detecting an optical signal from the labeled cellular bioparticles; and analyzing the optical signal to characterize at least one quality or quantity parameter of the labeled cellular bioparticles. The method may additionally comprise comparing the optical signal obtained in the steps above to an optical signal obtained with a control sample of identically labeled cellular bioparticles that are not subject to any cell proliferation stimulus.

In some embodiments, a method of analysis of a bioparticle may comprise immunophenotyping analysis which further includes labeling the bioparticles with one or more conjugated antibodies prior to the acoustic focusing in the method steps described above. In these embodiments, certain optical signals are indicative of a particular immunophenotype. In some embodiments, the labeled bioparticles are cells which are labeled with multiple conjugated antibodies. Any cell type can be immunophenotyped by the present methods. In some embodiments, cells that can be immunophenotyped by the present methods include blood cells such as human blood cells. In some embodiments, human blood cells may be immunophenotyped based on the expression of an antigenic marker such as but not limited to a CD45 marker, a CD3 marker, a CD4 marker, a CD8 marker, a CD19 marker and/or a CD56 marker. In some embodiments, a human blood cell can be immunophenotyped into cellular groups such as T-cells, B-cells, NK-cells, CD3 T-cells, CD19B-Cells, CD56-NK cells, CD4 T-helper cells and/or CD8 T-suppressor cells lymphocytes. In some embodiments, the method further comprises performing a multi-color immunophenotyping to simultaneously immunophenotype multiple cell populations into different immunophenotypes. In one example embodiment, six-color immunophenotyping can be performed simultaneously.

Some embodiments describe methods for detecting phosphoproteins on a cell (a cellular bioparticle) disposed within a fluid medium, comprising: stimulating or inhibiting the cell with a kinase or a kinase inhibitor respectively to phorsporylate or de-phosphorylate one or more proteins on the cell; contacting the cell with one or more antibody specific to detect the one or more phosphorylated protein; acoustically focusing the cell in the fluid medium; optically exciting the cell with an excitation source; detecting an optical signal from the cell; and analyzing the optical signal, wherein the optical signal is indicative of the presence or absence of the one or more phosphorylated protein. Several phosphorylated proteins determine the metabolic and pathological status of cells since phosphorylation and de-phosphorylation of these proteins are triggers of several cell signaling pathways.

Some embodiments describe methods for detecting fluorescent protein expression on a cell disposed within a fluid medium, comprising: transfecting the cell with one or more fluorescent proteins; acoustically focusing the cell in the fluid medium; optically exciting the cell with an excitation source; detecting one or more optical signals from the cell; and analyzing the optical signal, wherein the detection of an optical signal corresponding to one or more fluorescent protein is indicative of the presence of expression of the one or more fluorescent proteins and the absence of an optical signal corresponding to one or more fluorescent protein is indicative of the absence of expression of the fluorescent protein. In some embodiments, detection of an optical signal corresponding to one or more fluorescent protein is indicative of successful transfection. Accordingly, this method may also be a method of detecting successful transfection of a construct and/or a method of detecting successful protein expression in a system.

In some embodiments, the method may be further used to analyze transfection and/or expression of two proteins, wherein the detection of a first optical signal corresponding to a first fluorescent protein and the detection of a second optical signal corresponding to a second fluorescent protein is indicative of transfection of the cell by the first and the second fluorescent proteins. In some embodiments, analyzing an optical signal may further comprise analyzing the percentage of cells transfected with the one or more fluorescent proteins to quantify the number of transfected cells. Some embodiments describe methods comprising acoustically focusing one or more bioparticles expressing or co-expressing one or more fluoroscent proteins. Detection of any fluorescent protein is possible by the present methods and some example fluorescent proteins include, but are not limited to, a red fluorescent protein, a green fluorescent protein, a blue fluorescent protein and/or a yellow fluorescent protein.

In some embodiments, the disclosure describes methods for detection a rare event within a population of cells, comprising: acoustically focusing the population of cells; optically exciting the population of cells with an excitation source; detecting one or more optical signals from the population of cells; and analyzing the optical signal, wherein the detection of an optical signal corresponding to a rare event is indicative of the presence of the rare event and the absence of an optical signal corresponding to a rare event is indicative of the absence of the rare event. In some embodiments, a rare event can comprise detection of a rare subset of cells within the population of cells. In some embodiments, a rare subset of cells can comprise: less than 5% the population of cells or less than 0.5% the population of cells or from about 1 to about 20 cells in a 1 milliliter (ml) sample volume. Exemplary rare subsets of cells that may be detected by the methods described here include plasmocytoid dendritic cells, CD34+ cells from a population of peripheral blood cell, human mesenchymal cells, angiogenic cells, circulating endothelial cells and circulating hematopoietic progenitor cells in human blood. Some example embodiments comprise a method for detecting rare events, comprising acoustically focusing one or more bioparticles including at least one of a plasmacytoid dendritic cell, a circulating endothelial cell, a human mesenchymal cell and/or a CD34⁺ cell. In some embodiments, a method of the disclosure may additionally comprise identification of the rare subset of cells such as by phenotyping, immunophenotyping, determining protein expression, protein phosphorylation status, cell phase status etc.

In some embodiments, methods of analysis may comprise analyzing the cell cycle phase of a cell and comprise acoustically focusing one or more labeled cellular bioparticles through an interrogation zone; optically exciting the one or more labeled cellular bioparticles in the interrogation zone with an excitation source; detecting an optical signal from the labeled cellular bioparticles; and analyzing the optical signal to characterize at least one quality or quantity parameter of the labeled cellular bioparticles, wherein different optical signals correspond to different cell cycle phases. Some embodiments may further comprise steps for quantitating the percentage of cells (cellular bioparticles) in one or more cell cycle phases comprising additional analysis of the optical signal to quantify the different cell cycle phase signals to determine the number of cells in a particular cell cycle phase.

In some embodiments, methods of analysis comprise analyzing a microbe comprising acoustically focusing one or more microbial bioparticles through an interrogation zone; optically exciting the one or more microbial bioparticles in the interrogation zone with an excitation source; detecting an optical signal from the microbial bioparticles; and analyzing the optical signal to characterize at least one quality or quantity parameter of the microbial bioparticles, wherein different optical signals correspond to different types of microbial events. Detection of one or more optical signals are indicative of microbial cell events such as microbial viability, number of microbial cells, detection of gram positive status of a microbe, detection of gram negative status of a microbe, microbial membrane potential, microbial metabolism and combinations thereof. In some embodiments, microbial viability comprises detecting live microbial cells separately from dead microbial cells. In some embodiments the method comprises analyzing a prokaryotic cell such as a bacterial cell, a picophytoplankton cell using an acoustic focusing cytometer.

In some embodiments, methods of the disclosure comprise detecting apoptosis in a cell comprising: acoustically focusing one or more cells disposed within a fluid; optically exciting the one or more cells with an excitation source; detecting one or more optical signals from the cells; and analyzing the detected optical signals to identify morphological or biochemical changes that are indicative of cell apoptosis. An optical signal corresponding to detecting an apoptotic event in the cell is indicative of an apoptotic cell and the absence of an optical signal corresponding to detecting an apoptotic event in the cell is indicative of the absence of apoptosis. A variety of optical signal may correspond to detecting an apoptotic event and may include examples such as but not limited to detecting a change in the cells mitochondrial membrane potential, a change in the cells mitochondrial redox potential, a change in the protein composition in the cells plasma membrane, translocation of cellular and/or membrane components (proteins, lipids, nucleic acids) and combinations thereof. In one example embodiment, an optical signal corresponding to detecting translocation of phosphatidylserine (PS) from the inner leaflet of the plasma membrane of the cell to the outermembrane of the plasma membrane of the cell is indicative of an apoptotic cell.

Some embodiments describe methods for analysis of a labeled bioparticle comprising: performing a no-lyse and/or a no-wash cell preparation step prior to the analysis to minimize sample loss. Such methods are especially important for low volume samples and hard to obtain samples. Analysis may comprise immunophenotyping a cell, cell cycle analysis, rare event detection, detection of fluorescence, detection of transfection, detection of protein expression.

In some embodiments, the disclosure describes a computer program product comprising computer readable instructions, which, when executed by a computer in or in communication with an acoustic focusing cytometer, are configured to perform one or more of the steps embodied in any one or more of the methods described herein. The disclosure also describes an apparatus and/or a system comprising a computer and a controller configured to control the acoustic focusing of particles and to perform one or more of the steps embodied in any one of the methods for analyzing bioparticles as described herein.

Another embodiment of the present disclosure comprises a kit for acoustically focusing and analyzing at least one bioparticle. This kit preferably includes in one or more container means one or more of the following: a population of bioparticles, a container means having a reagent for labeling the bioparticles, a labeled bioparticle population, reagents and/or buffers and other compositions needed to perform acoustic focusing and analysis of the labeled bioparticle.

### BRIEF DESCRIPTION OF THE DRAWINGS

The accompanying drawings, which are incorporated into and form a part of the specification, illustrate one or more embodiments of the present disclosure and, together with the description, serve to explain the principles of the disclosure. The drawings are only for the purpose of illustrating one or more preferred embodiments of the disclosure and are not to be construed as limiting the disclosure. In the drawings:

FIG. 1 is an illustration of field focused particles according to various embodiments.

FIG. 2 is an illustration of a single line acoustic focusing device according to various embodiments.

FIG. 3 illustrates a schematic of an acoustically driven flow cell focusing particles to the center of a flowing liquid stream across laminar flow lines according to various embodiments.

FIG. 4 illustrates one embodiment of an acoustically driven flow cell with two laminar flow streams in contact.

FIG. 5 illustrates the separation of micron sized polystyrene fluorescent orange/red particles from a background of nanometer sized green particles in a homogeneous fluid according to various embodiments.

FIG. 6 illustrates particle separation across laminar flow boundaries for particles of different size according to various embodiments.

FIG. 7 illustrates multiple embodiments of analysis in a flow cytometer like configuration for particles.

FIG. 8 illustrates a schematic of an acoustically focused flow cell in combination with an acoustic flow cytometer according to various embodiments.

FIG. 9 illustrates a flow diagram according to various embodiments.

FIG. 10 illustrates the flow diagram in FIG. 9 modified to include in-line laminar washing according to various embodiments.

FIGS. 11A and 11B illustrate field focusing of laminar wash fluid according to various embodiments.

FIG. 12 illustrates a schematic of parallel fluid switching device according to various embodiments.

FIG. 13 is a schematic for stream switching of unlysed whole blood according to various embodiments.

FIG. 14 is a schematic of an acoustic stream switching particle impedance analyzer according to various embodiments.

FIG. 15 is a schematic example of separation of negative contrast carrier particles from a core of blood sample according to various embodiments.

FIG. 16 illustrates a schematic example of multi-plexed competitive immunoassaying in an acoustic wash system according to various embodiments.

FIG. 17 illustrates a flow chart for high throughput/high content screening using acoustic fluid switching according to various embodiments.

FIG. 18 illustrates a schematic example of a two chamber culturing/harvesting vessel using acoustic washing to harvest a spent medium and place cells in a fresh medium according to various embodiments.

FIG. 19 illustrates a diagram of an aptamer selection from a library, multiplexed beads or cells with target molecules incubated with aptamer library according to various embodiments.

FIG. 20 illustrates an example of a dual stage acoustic valve sorter according to various embodiments.

FIG. 21 illustrates a system and method for optical analysis of acoustically repositioned particles and a medium.

FIG. 22 illustrates a diagram of particle groupings with different parameters.

FIG. 23 illustrates an image of acoustically repositioned particles imaged by an imager.

FIG. 24 illustrates acoustic positioning of particles for fusion or reaction.

FIG. 25 illustrates a first acoustic focuser focusing particles in a tight, single file line and then a second acoustic focuser separating particles based on size.

FIGS. 26A and 26B depict acoustic focusing fluorescent microsphere beads and depict beads flowing through prior to acoustic focusing (left panel FIG. 26A), i.e., acoustic focusing is off and the sample is unfocused and after acoustic focusing beads focused into a single line (right panel, FIG. 26B), i.e., acoustic focusing is on and the sample is focused, according to one embodiment.

FIGS. 27A and 27B depicts acoustic focusing vs. traditional hydrodynamic focusing, according to various embodiments.

FIG. 28 depicts is a block diagram that illustrates a computer system 700 that may be employed to carry out a method of the disclosure, according to some exemplary embodiments of the disclosure.

### DETAILED DESCRIPTION

This disclosure relates to systems using acoustic radiation pressure. Acoustic radiation pressure can be used to concentrate and align particles in fluids. This ability has many applications in the fields of particle analysis and sample preparation. As described herein acoustic radiation pressure is applied primarily to flow cytometry, reagents for use in flow cytometry and sample preparation for flow cytometry. Acoustic cytometers, using relatively large dimension flow channels, concentrate particles from the entire volume of the channel to a small acoustic trap in the center of the channel and can therefore offer both controllable flow and high particle analysis rates without requiring highly concentrated samples. Many of the sample preparation methods have wider application and a few of these embodiments are disclosed.

As used herein “acoustic contrast” means the relative difference in material properties of two objects with regard to the ability to manipulate their positions with acoustic radiation pressure. The acoustic force due to acoustic radiation pressure on a compressible, spherical particle of volume V in an arbitrary acoustic field (neglecting viscosity and thermal conductivity) can be written in terms of an acoustic radiation pressure force potential U:

$$U = \frac{4}{3} π {a}^{3} [ ( {β}_{o} \frac{〈 {p}^{2} 〉}{2} ) {f}_{1} - \frac{3}{2} ( \frac{{ρ}_{0} 〈 {v}^{2} 〉}{2} ) {f}_{2} ] . \qquad ( 1 )$$

Here, a is the particle radius, β₀ is the compressibility of the surrounding fluid, and ρ₀ is the density of the surrounding fluid. The pressure and velocity of the acoustic field in the absence of the particle are described by p and v, respectively, and the brackets correspond to a time-averaged quantity. The terms f₁ and f₂ are the contrast terms that determine how the mechanical properties (compressibility and density) of the particle differ from the background medium. They are given by:

$$\begin{matrix}{f}_{1} = 1 - \frac{{β}_{p}}{{β}_{o}} & ( 2 a ) \\ {f}_{2} = \frac{2 ( {ρ}_{p} - {ρ}_{o} )}{( 2 {ρ}_{p} + {ρ}_{o} )} & ( 2 b )\end{matrix}$$

The subscript p corresponds to intrinsic properties of the particle. The force F acting on a particle is related to the gradient of the force potential U by:

F=−∇U (3)

Particles will be localized at positions where the potential U displays a minimum (stable equilibrium). The acoustic contrast of a particle (or medium or fluid) is determined by the density and compressibility differences between it and the background medium or fluid as defined by terms f₁ and f₂ in Eqs. 2a and 2b. The relative magnitudes and signs of f₁ and f₂ determine the behavior of the radiation force potential U and thus determine the magnitude and direction of the acoustic radiation pressure force. As an example, if a particle and the background medium or fluid share the same density value (ρₚ=ρ₀), then f₂ is zero and the acoustic contrast is due only to compressibility differences in f₁. If both f₁ and f₂ are zero, then acoustic contrast is zero. Viscosity and thermal conductivity will also have effects on acoustic contrast, but theses are widely neglected in the literature. Equation 1 is generally sufficient to describe the acoustic contrast relationship for most samples of interest.

As used herein “a” means one or more.

As used herein “assaying” means a method for interrogating one or more particles or one or more fluids.

As used herein “assay” “method” or “protocol” means a product, including but not limited to, a list of steps of a method, a workflow, an assay kit, data and/or report.

As used herein “flow cell” means a channel, chamber or capillary having an interior shape selected from rectangular, square, elliptical, oblate circular, round, octagonal, heptagonal, hexagonal, pentagonal, and triagonal.

As used herein “channel” means a course, pathway, or conduit with at least an inlet and preferably an outlet that can contain an amount of fluid having an interior shape selected from rectangular, square, elliptical, oblate circular, round, octagonal, heptagonal, hexagonal, pentagonal, and triagonal.

As used herein “acoustically focusing”, “acoustically focused”, “acoustically focuses” and “acoustic focusing” means the act of positioning particles within a flow cell by means of an acoustic field. An example of acoustic focusing of particles is the alignment of particles along an axis of a channel. The spatial extent of the focal region where particles are localized is determined by the flow cell geometry, acoustic field, and acoustic contrast. As viewed in the cross sectional plane of a flow cell, the shape of observed focal region can resemble a regular geometric shape (e.g. point, line, arc, ellipse, etc.) or be arbitrary. The primary force used to position the objects is acoustic radiation pressure. The acoustic systems of the present disclosure are sometimes referred to herein as flow cytometers, acoustic cytometers, flow cells or long transit time devices, however all such systems have acoustic radiation pressure.

As used herein “acoustically reorienting” and “acoustically reorients” means the act of repositioning the location of miscible, partially miscible, or immiscible laminar flow streams of fluid or medium within a device with acoustic radiation pressure. This technique utilizes differences in the mechanical properties (acoustic contrast) of separate laminar streams in a flow channel. When two fluids are brought into contact, a large concentration gradient can exist due to differences in their molecular makeup's resulting in an interfacial density and/or compressibility gradient (acoustic contrast between streams). For diffusion, time scales that are larger than the time scales of the acoustic excitation, the laminar flow streams can be acted upon with acoustic radiation pressure. Under the action of the acoustic field, the streams are reoriented within the flow cell with an acoustic field based upon their acoustic contrast.

As used herein “particle” means a small unit of matter, to include but not limited to: biological cells, such as, eukaryotic and prokaryotic cells, archaea, bacteria, mold, plant cells, yeast, protozoa, ameba, protists, animal cells; cell organelles; organic/inorganic elements or molecules; microspheres; and droplets of immiscible fluid such as oil in water.

As used herein a “bioparticle” means a particle or molecule of biological origin and may include without limitation a cell (any cell), a cell organelle, a protein, a peptide, a nucleic acid and/or a virus.

As used herein “analyte” means a substance or material to be analyzed.

As used herein “probe” means a substance that is labeled or otherwise marked and used to detect or identify another substance in a fluid or sample.

As used herein “target” means a binding portion of a probe.

As used herein “reagent” is a substance known to react in a specific way.

As used herein “microsphere” or “bead” means a particle having acoustic contrast that can be symmetric as in a sphere, asymmetric as in a dumbbell shape or a macromolecule having no symmetry. Examples of microspheres or beads include, but are not limited to, silica, glass and hollow glass, latex, silicone rubbers, polymers such as polystyrene, polymethylmethacrylate, polymethylenemelamine, polyacrylonitrile, polymethylacrylonitrile, poly(vinilidene chloride-co-acrylonitrile), and polylactide.

As used herein “label” means an identifiable substance, such as a dye, a fluorescent molecule, or a radioactive isotope that is introduced in a system, such as a biological system, and can be followed through the course of a flow cell or channel, providing information on the particles or targets in the flow cell or channel.

As used herein “signaling molecule” means an identifiable substance, such as a dye or a radioactive isotope that is introduced in a system, such as a biological system, and can be used as a signal for particles.

As used herein “inherently axially symmetric” means an object that displays a high degree of axial symmetry. Examples of inherently axially symmetric geometries include oblate circular cross section cylinders, elliptical cross section cylinders, and oval cross section cylinders, but not limited thereto.

Field based focusing of particles via magnetic fields, optical fields, electric fields and acoustic fields, enables the localization of particles without the need for sheath fluid. Focused particles can be flowed past interrogating light sources at whatever linear velocity is chosen using an adjustable external pumping system such as a syringe pump. Field based focusing also concentrates particles in the medium which allows for high particle analysis rates without the need to pre-concentrate samples. Field based focusing for flow cytometry, where particles are analyzed one by one, has been accomplished with dielectrophoretic and acoustic systems. This can be done using other fields, such as magnetic fields, optical fields or electrophoretic fields.

Field based focusing of particles relies on contrasts in physical properties between the particle being focused and the medium. For dielectrophoretic focusing, this relies on dielectric properties. For magnetic focusing, magnetic susceptibility and for acoustic manipulation this relies on acoustic properties, primarily density and compressibility.

Magnetic focusing of cells typically requires binding of magnetic material to the cells and dielectrophoretic focusing typically requires careful control of the media conductivity as well as very small dimensions for high field gradients. This makes acoustic focusing particularly attractive for many analytes as it typically does not require reagents to change the contrast of particles and can be performed in relatively large dimension channels with complex one or more media of highly variable conductivity and or pH.

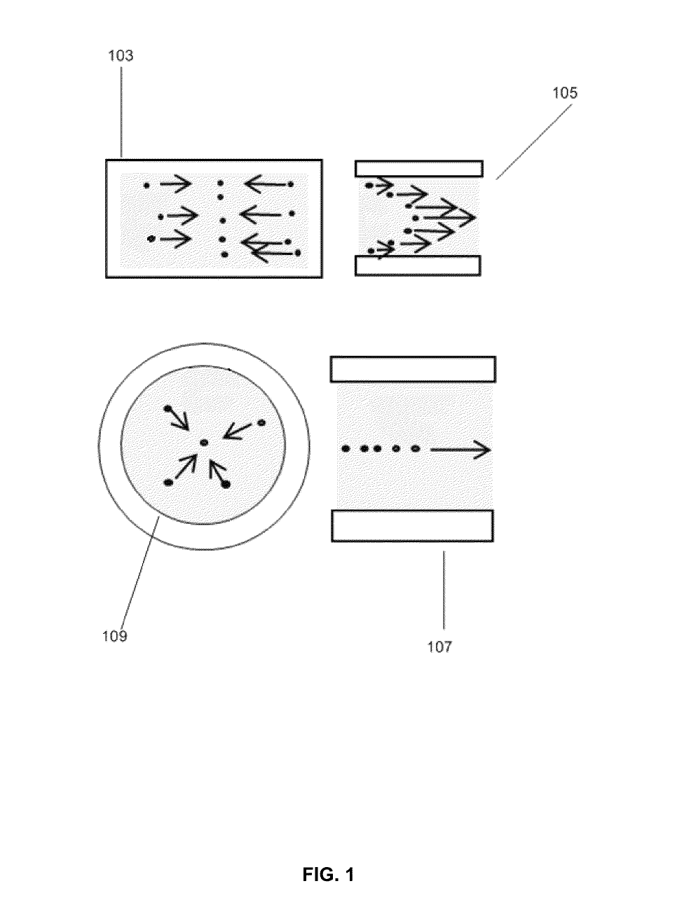

Referring now to FIG. 1, a schematic comparison of planar microchannel focusing 103 and 105 and line driven capillary focusing 107 and 109. Planar focusing results in a two dimensional sheet of particles 102 with varying velocities arrows along the flow direction. Cylindrical line drive focusing places particles 102 in the center where they travel at the same rate single arrow.

Particle Manipulation in Acoustically Driven Capillaries

To calculate the acoustic force on particles within an ultrasonic standing wave, the acoustic radiation pressure force on a compressible, spherical particle of volume V in an arbitrary acoustic field can be written in terms of an acoustic radiation pressure force potential U (Gor'kov 1962):

$$U = \frac{4}{3} π {a}^{3} [ ( {β}_{o} \frac{〈 {p}^{2} 〉}{2} ) {f}_{1} - \frac{3}{2} ( \frac{{ρ}_{0} 〈 {v}^{2} 〉}{2} ) {f}_{2} ] . \qquad ( 1 )$$

Here, a is the particle radius, β₀ is the compressibility of the surrounding fluid, and ρ₀ is the density of the surrounding fluid. The pressure and velocity of the acoustic field in the absence of the particle are described by p and v, respectively, and the brackets correspond to a time-averaged quantity. The terms f₁ and f₂ are the contrast terms that determine how the mechanical properties of the particle differ from the background medium. They are given by:

$$\begin{matrix}{f}_{1} = 1 - \frac{{β}_{p}}{{β}_{o}} & ( 2 a ) \\ {f}_{2} = \frac{2 ( {ρ}_{p} - {ρ}_{o} )}{( 2 {ρ}_{p} + {ρ}_{o} )} & ( 2 b )\end{matrix}$$

The subscript p corresponds to intrinsic properties of the particle. The force F acting on a particle is related to the gradient of the force potential U by:

F=−∇U (3)

Particles will be localized at positions where the potential U displays a minimum.

According to various embodiments disclosed herein a round or oblate cross-section capillary that acoustically focuses particles either along the axis of the capillary or along the capillary wall is tuned with an acoustic wave. The position of the particle within the capillary depends upon the value of its density and compressibility relative to the background medium as shown in the acoustic contrast terms f₁ and f₂ above.

A cylindrical geometry according to various embodiments disclosed herein creates a radial force profile with radial restoring forces that hold the particles in a single stream line along the axis of the flow. This affords single file particle alignment along the axis of the capillary while using only a single acoustic excitation source. There are several benefits identified with the cylindrical (inherently axially symmetric geometries such as oblate cylinders, ellipses, etc.) for particle manipulation. The benefits include: higher throughput on the order of several ml/min versus several hundred μl/min per fluid channel. Fine positioning of blood cells along the axis of a capillary at flow rates of 0 to 5 mL/minute have been achieved in 340 micron diameter capillaries.

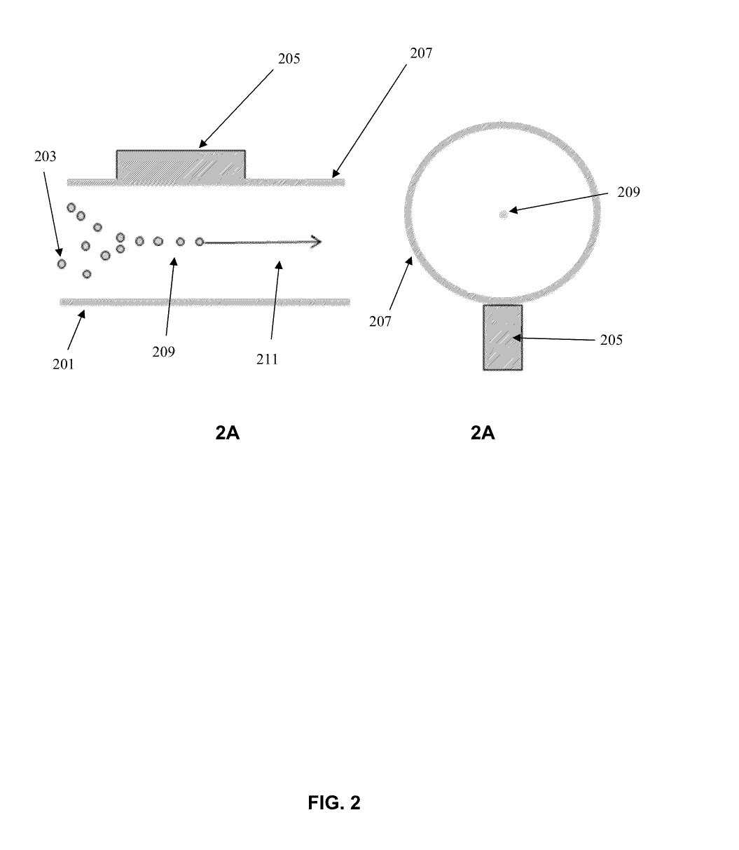

Referring now to FIG. 2, a single line acoustic focusing apparatus is illustrated according to various embodiments. FIG. 2 gives a side view A and axial view B of a cylindrical tube 201 acoustic focusing device that acoustically focuses particles 203 to a pressure minimum in the center of the tube 209 by transducer 205. The stream of particles is sent to analysis 211. Analysis includes any post focusing interrogation or further processing. The flow cell is not limited to a tube or a cylindrical shape.

The particles are maintained in a single velocity stream line that allows uniform residence time for similar size and acoustic contrast particles. This is important for any process for which reaction kinetics are important.

Radial force driven acoustic focusing of particles coupled with tight central focusing of a light source on the particles allows analysis of particles one by one as in a flow cytometer and simultaneous concentration of the particles. This type of analysis is much more powerful than a simple fluorescent readout step as it allows multiplexed identification and quantification of each particle/assay as well as single particle statistics.

### Acoustic Manipulation of Background Media

According to another embodiment, a method for acoustically reorienting a medium provides that the medium within the device is acoustically manipulated in addition to the position of the particles. This embodiment utilizes differences in the mechanical properties (acoustic contrast) of separate laminar streams in a flow channel. As used herein, medium is used interchangeable with fluid.

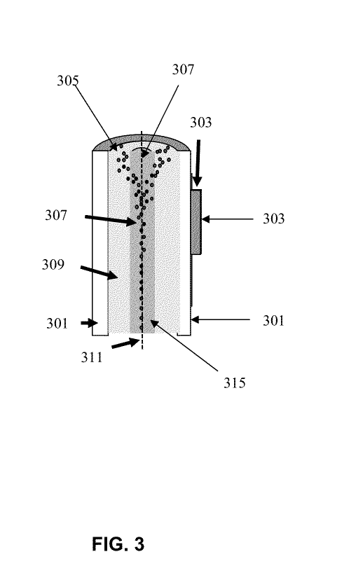

Referring now to FIG. 3, a schematic of a line driven capillary 301 acoustically focusing particles to the center of a flowing fluid stream 311 comprising clean fluid 307 as the particles move across laminar flow lines 315 is illustrated according to various embodiments. Particles in sample 305 are acoustically focused from sample stream 309 and can be tightly acoustically focused for single file analysis. Wash buffer 307 provides fluid stream in which particles are finally contained. Transducer 330 provides acoustic standing wave.

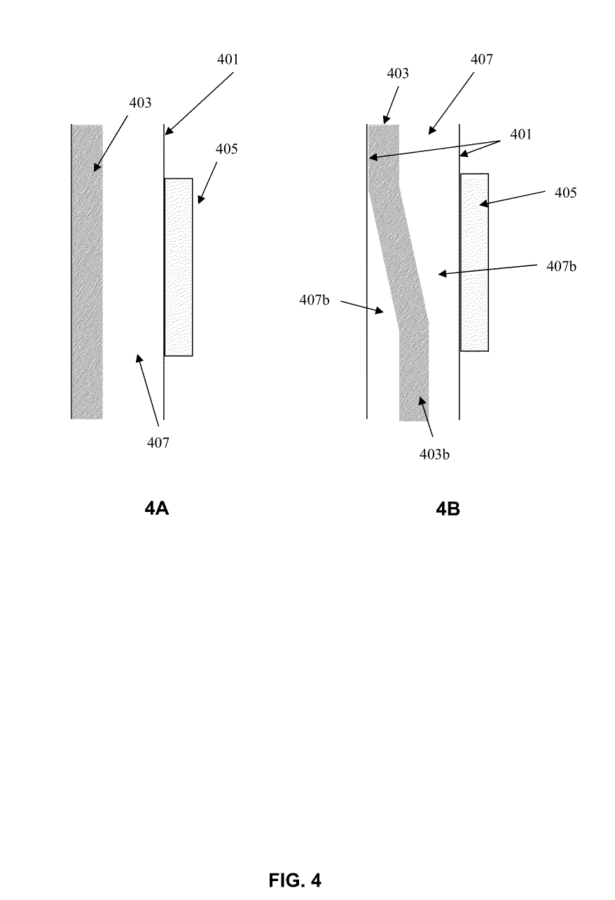

Referring now to FIG. 4, one embodiment of an acoustically driven capillary 401 and 405 with two laminar flow streams 403 and 407 in contact is illustrated in FIG. 4A. Upon activation of the acoustic field in FIG. 4B, the positions of the fluid streams are acoustically reoriented based upon the acoustic contrast of each stream. In one embodiment, the flow stream with greater acoustic contrast 403b is reoriented to the center of the acoustically driven focused capillary while the flow stream with lower acoustic contrast 407b is acoustically reoriented near the capillary walls. In FIG. 4A, the acoustic field is OFF and streams flow parallel down the channel. As illustrated in FIG. 4B, when the acoustic device is activated in a dipole mode, Stream 1 403a moves coincident with the central axis of the capillary partially displacing Stream 2 407b. Equations 1-3 approximate the stream that is more dense and/or less compressible is forced to the central axis position. Flow direction is downward on the plane of the page.

Equations (Eqs.) 1 and 2 describe an acoustic contrast that predicts the magnitude and direction of the acoustic radiation pressure force on particles in a fluid or medium. The force depends upon the differences in the density and/or compressibility of a particle relative to the density and/or compressibility of the background medium. Although this type of effect has traditionally been used to study particles, emulsions, and bubbles in fluids, it has also been applied to extended objects in fluids. For example, the acoustic radiation pressure force has been shown to effectively stabilize liquid bridges of silicone oil in water. It was observed that liquid bridges density-matched to a background water medium can be driven with modulated acoustic radiation pressure. The force results from a difference in the compressibilities (acoustic contrast) of the liquid bridge and background medium. Similarly, experiments using air as a background medium have proven the acoustic radiation force is effective for the manipulation of both small diffusion flames of natural gas and dense gases surrounded by air.

The effect shown in FIG. 4 takes advantage of differences in the composition of the laminar flow streams. The streams can be immiscible, partially-miscible, or miscible. When two fluids are brought into contact, a large concentration gradient can exist due to differences in their molecular makeups. For immiscible fluids, this interface is assumed to be infinitely narrow. For miscible fluids, the concentration gradient is a transient interfacial phenomena that relaxes over time due to diffusion and other transport mechanisms. For the description of acoustic processes, the concentration gradient is viewed as a density and/or compressibility gradient. For diffusion time scales that are much larger than the time scales of an acoustic excitation, the laminar flow streams can be considered isolated entities with different densities and compressibilities (acoustic contrast) that can be acted upon with acoustic radiation pressure. Multiple laminar stream systems have been developed where the flow streams are manipulated consistent with the density and compressibility relationships shown in Eqs. 1 and 2. Examples of these systems are illustrated herein.

It should be noted that Eq. 1 is approximated in the long wavelength limit, where it is assumed that the particle acted upon by the acoustic radiation pressure force is much smaller than the wavelength of sound excitation (λ>>a). It also ignores multiple scattering from the particle. (Contributions from wave reflections at the media interfaces to the resident acoustic field can also become considerable as the acoustic contrast between streams increases.) For this reason, it is assumed that Eq. 1 is not an exact description of the interaction of the acoustic field with the laminar flow streams in the devices described here. Acoustic radiation pressure induced manipulation of miscible laminar flow streams of diameter b in the limit where λ>>b is upheld, as well as for larger diameter streams where λ˜4 b, have been observed. Equations 1-2 serve as qualitative predictors for the location of the final stream position by defining a relationship between the relative density and compressibility of the streams within the flow channel. Corrections to the final shape of the streams due to shaping associated with acoustic radiation pressure and gravity will affect their final cross sectional geometry within the cavity, but the approximate position of the stream is still predicted by density and compressibility contrasts (acoustic contrast).

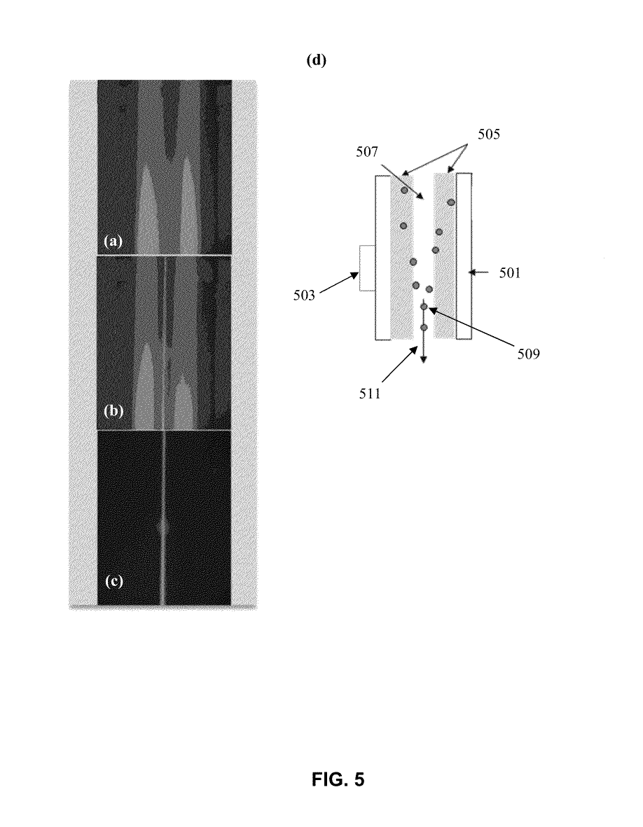

FIG. 5 illustrates the separation of micron sized polystyrene fluorescent orange/red particles from a background of nanometer sized green particles in homogeneous media according to various embodiments. The time-averaged acoustic force scales with the volume of a cell/particle. Because of this it is possible to fractionate particles not only by acoustic contrast to the media but also by size. By flowing a clean stream in the radial center of a separation device however, it is possible to prevent the smaller particles from reaching the center before the point of axial particle collection. Furthermore, if the center stream has higher specific gravity and/or lower compressibility than the outer sample stream, the particles/cells with greater acoustic contrast than the center wash fluid will continue to focus to the capillary axis while particles/cells of lesser contrast will be excluded.

Referring now to FIG. 5a, polysciences fluoresbrite polychromatic red 5.7 μm latex particles are mixed with Polysciences 200 nm fluoresbrite green particles in the coaxial stream. A particle stream flowing through the capillary under epi-fluorescent illumination (FITC long-pass filter) with acoustic field off is illustrated. FIG. 5b is an activation of the acoustic field that acoustically focuses the 5.7 μm particles (which fluoresce yellow under blue illumination) to a line along the central axis of the capillary, leaving the 200 nm particles not acoustically focused and remain in their original flow stream. The 5.7 μm particles are like particles with like acoustic contrast. FIG. 5c shows green illumination with red band pass filter. The 5.7 μm particles fluoresce red while the 200 nm particles are not excited. FIG. 5d illustrates clean core stream 507 introduced alongside coaxial stream containing fluorescent background 505. Transducer 503 includes acoustic standing wave (not shown). Particles 509 are acoustically focused upon entering standing wave. The acoustically focused particles cross from sample stream 509 to core stream 507 and thereby are removed from sample fluid.

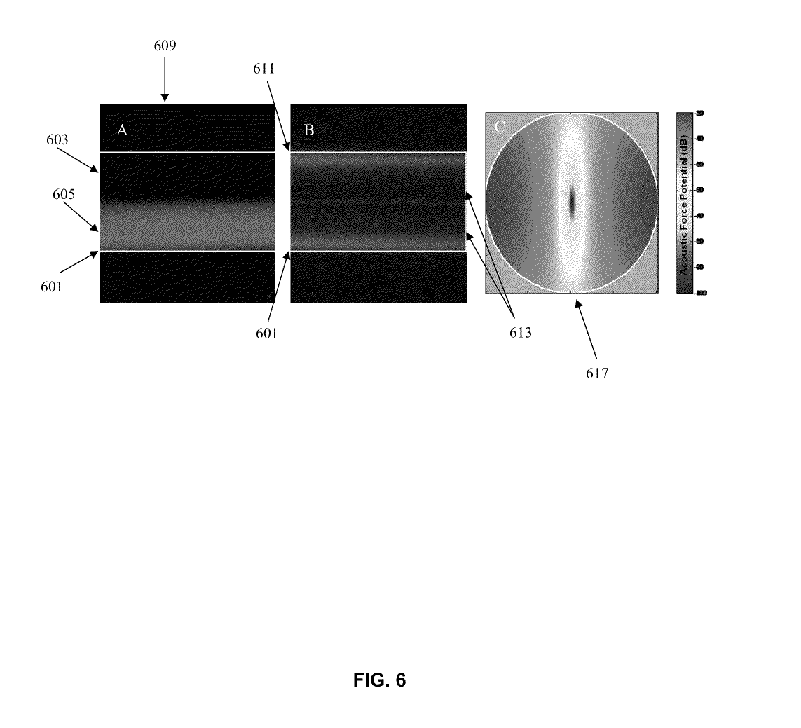

Referring now to FIG. 6, particle acoustic focusing of particles across laminar streams and acoustic reorientation of medium is illustrated. FIG. 6A illustrates the fluorescence image of an optical cell coupled to the end of an acoustic focusing cell with acoustic field off. White lines are added to indicate edges of 250 μm flow cell. Excitation light passes through a 460 nm bandpass filter and emission is filtered through a 510 nm long-pass filter. Flowing through the bottom half of the flow cell is a mixture of 10% whole blood in PBS buffer spiked with 25 μg/ml of R-Phycoerythrin fluorescent protein (orange fluorescence). White blood cell DNA is stained with SYTOX green. At the top is 6% iodixanol in PBS buffer (dark).

FIG. 6B illustrates the same optical cell and media after acoustic field is turned on. The 6% iodixanol in PBS buffer acoustically reorients to center while the PBS/PE/blood plasma mixture is acoustically reoriented toward both the left and right sides of the cell (top and bottom in the figure). The white cells leave their original medium and are acoustically focused to the center where they are observed as a green line. Red cells also acoustically focus to this location but are not visible in the fluorescent image.

FIG. 6C illustrates MATLAB plot of the approximate acoustic force potential (Eqs. 1 and 2) for particles that are more dense/less compressible than the background. This is an axial view of an acoustically driven capillary with an extended source aperture (flow is into the page). More dense, less compressible particles/media e.g. cells and iodixanol/PBS medium, are acoustically focused/acoustically reoriented toward the center (dark blue region) and less dense and/or more compressible media e.g. PBS/PE/blood plasma mixture are acoustically focused/acoustically reoriented toward the left and right sides (dark red regions).

When separating particles or cells using heterogeneous media in which the wash stream fluid's specific gravity and/or compressibility (acoustic contrast) differs from that of the sample fluid stream, the separate laminar streams can be affected by the acoustic field. For example, if blood cells are to be separated from the protein in serum and the wash stream has higher specific gravity/lower compressibility, then the entire sample stream is pushed toward the center of the fluid cavity (e.g. capillary axis in an acoustically driven capillary). This condition is met when even very dilute blood in physiological saline is the sample stream and physiological saline is the wash stream. If however, the wash stream is made to have higher specific gravity/lower compressibility than the sample stream, but lower specific gravity/higher compressibility than the cells, the wash remains in the central core, the cells move toward the center of the cavity and the sample medium is pushed to the sides.

Modeling for the acoustic field distribution shows where the sample media should approximately be positioned for the case described above (FIG. 6C). Using Eq. 1-3, a potential minimum exists in the center of the capillary for particles (or approximately for flow streams) that possess higher specific gravity/lower compressibility. Conversely, particles (or flow streams) that possess lower specific gravity/higher compressibility will be positioned at the potential maxima in the figure as the sign is reversed in Eqs. 2. An interesting result occurs when a sample stream of lower density (and/or higher compressibility) is flowed along the axial center of the capillary and a higher density (and/or lower compressibility) fluid stream is flowed adjacent to it. The streams will acoustically reorient to comply with the potential shown in FIG. 6C. This kind of stream separation has not been demonstrated or reported in planar systems.

The ability to place samples in the central core stream and still separate the sample fluid from the cells or particles can be used to increase throughput. The acoustic force is strongest near the minimum potential U in FIG. 6C and the distance the particle must travel to the minimum is minimized.

The data in FIG. 6 shows that phycoerythrin in the acoustically reoriented streams is positioned further from the center of the flow stream than with the acoustic field turned off. This may be used to advantage in an in-line system designed to exclude free antibody or other species, for example, particularly for slow flow rates/long residence times where diffusion might otherwise significantly penetrate the wash stream.

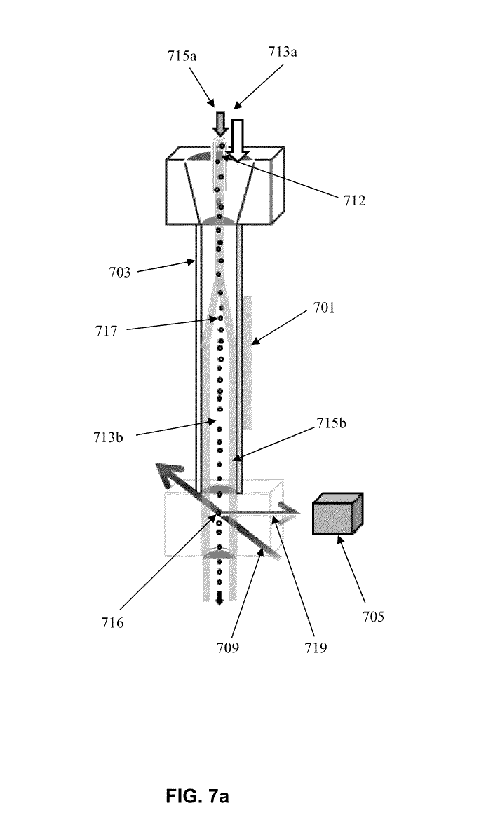

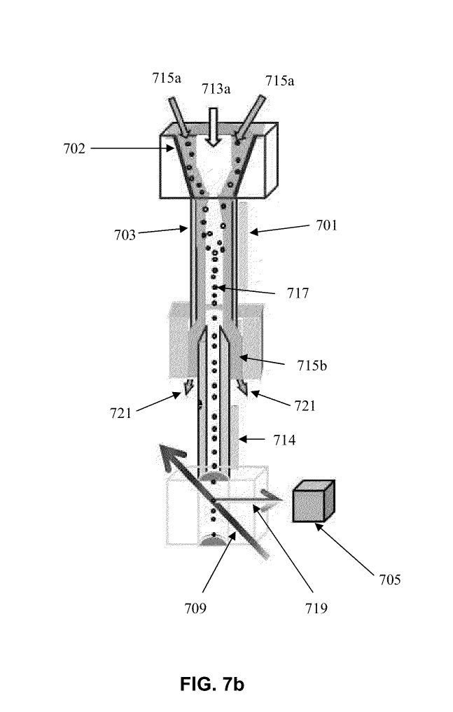

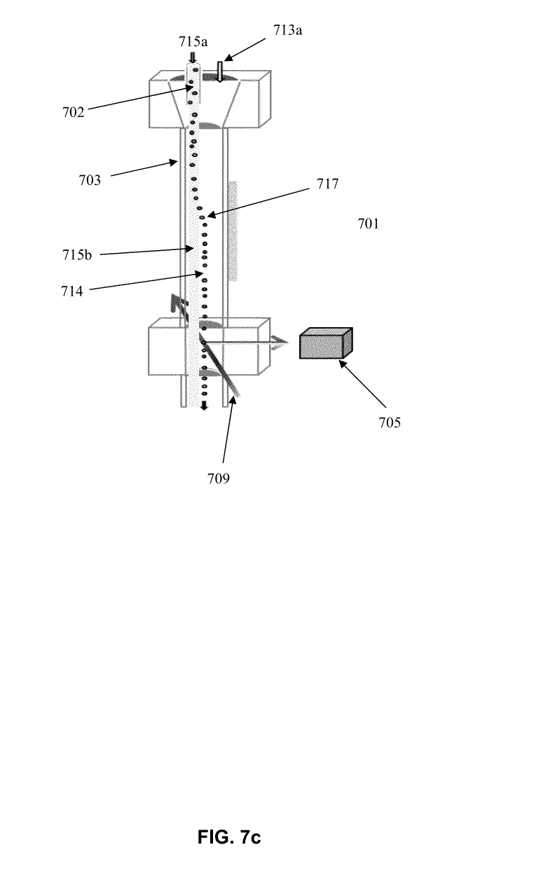

Referring now to FIG. 7, analysis in a flow cytometer like configuration where cells/particles are paraded through a tightly focused laser, illustrated such that the laser can be focused so that it does not excite the “dirty” media (FIG. 7A). Alternatively, the clean stream can be flowed independently through the optics cell (FIG. 7B). For this method clean media and target cells reach the detection region but the particles may need refocusing by a second focusing element. If the sample is injected slightly to one side of center and the stream is a small enough fraction of the total flow, the sample stream can be confined to one side of the optics cell (FIG. 7C). This is advantageous in flow cytometry for separating free vs. bound fluorophores in the analysis region.

Referring now to FIG. 7A, various embodiments comprise sample 715a which is introduced into the system alongside wash buffer 713a. Particles 712 in sample 715, sample 715 and wash buffer 713 are introduced into capillary 703a. A line drive 701 on capillary 703 introduces acoustic standing wave (not shown) naming a user defined mode (dipole mode is this example). The sample 715a and buffer 713a are acoustically reoriented and particles are acoustically focused based upon the acoustic contrast of each. Acoustically focused particles 717 are transited to an interrogation point 716 where laser 717 impinges electromagnetic radiation. An optical signal from the interrogated sample 719 is detected by the detector 705. The detector may be a PMT array for example.

Referring now to FIG. 7B, sample 715 comprising particle 702 is introduced into the system. The sample 715a, wash buffer 713a and particle are introduced into capillary 703. An acoustic wave (dipole mode) is induced into the capillary 703 by a first acoustic wave inducing means 701 such as a PZT drive but not limited thereto as other acoustic wave inducing means will produce same standing wave. Acoustic focusing of particles 702 cause each particle to be acoustically focused such that each particle having high enough acoustic contrast will focus in a line 717. Sample buffer with a lower concentration of particles after acoustic focusing will be discarded to waste 721 buffer 713 and 717 particle will be transited to a second acoustic wave inducing means 714. Particles are interrogated with a laser 709. The optical signal 719 for interrogated sample is sent to detector 705.

Referring now to FIG. 7C, sample 715 comprising particle 702 and buffer 713 are introduced into the system. Sample 715 flow next to capillary wall 703 and buffer 713a flows against the opposite wall. Transducer 717 induces acoustic wave that acoustically reorients sample 715b, acoustically focuses particles 714 and acoustically reorients buffer 713b. The particles 714 are transited to the interrogation point for interrogation of the particles 714 and buffer 713b by laser 709. The optical signal from interrogated particle 714 and/or buffer 713b is detected by detector 705. The velocity of the sample stream, buffer stream, particles is controlled by pumping system (not shown) such that the velocity is variable between 0 meters/second to 10 meters/second in the forward, reverse or stopped direction. Particles are washed in an acoustically reoriented first fluid which replaces the second fluid to produce washed particles.

One aspect of the various embodiments disclosed herein provides for an acoustic particle focusing technology in a cytometer that is capable of both high particle analysis rates up to 70,000 particles/second and/or capturing images from user selected subpopulations of cells.

Another aspect of the various embodiments disclosed herein provides for a system and method to analyze more than one hundred thousand cells per minute using traditional flow cytometry measurements and periodically adjust the velocity of the focused stream to collect images of only those cells that meet user defined criteria.

A further aspect of the various embodiments disclosed herein provides for a system and method wherein a first and a second fluid are acoustically reoriented and wherein the second fluid suppresses non-specific binding of a reagent that binds to a population of the particles.

Yet another aspect of the various embodiments disclosed herein provides for a system and method wherein particles are acoustically reoriented from a first fluid to a second fluid. The second fluid has a higher concentration of particles suspended therein after acoustically focusing the particles as compared to the second fluid prior to acoustically focusing the particles. Acoustically focusing the particles preferably creates a line of particles through about a center axis of a channel that flows parallel to an axis of flow.

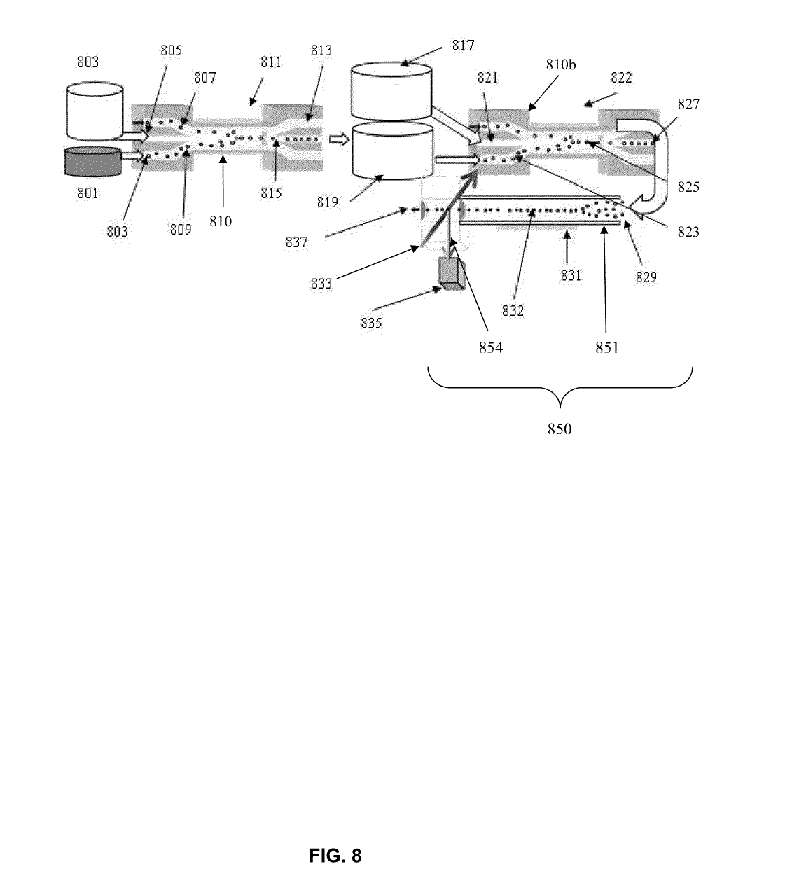

FIG. 8 illustrates various embodiments comprising a schematic of an acoustically focused flow cell for acoustically orienting particles and flow streams prior to collecting the acoustically focused sample. The sample 801 is introduced into a flow cytometer 850 that contains a transducer 831 for acoustically focusing particles 832 prior to analysis. Sample container 801 comprising sample particles 803, 807 and 809 is introduced to a flow cell 810. A transducer 811 provides acoustic wave to flow cell 810 to produced acoustically focused particle 815. Wash or other reagent 802 in introduced to flow cell 810. Particles 809 and 807 are acoustically focused into stream 805. The acoustically focused particle 815 exists with the wash stream 815 and is collected at collection/incubation site 819. Wash stream 821 is introduced from wash 817. Flow cell 810b with acoustic field generator 822 receives particles 823. The particles are acoustically focused 825 prior to introduction into the focus cytometer 850. A transducer 831 provides to flow cell 851 acoustic field and particles are acoustically focused 832 prior to reaching an interrogation point 852. Interrogation light 833 impinges on particle. A signal 854 from impinged particles is sent to detector 835 for analysis. The particle flows through system to point 837 for collection. In this embodiment, sample particles 803, 807 and 809 preferably have a particle acoustic contrast that is different from the acoustic contrast of sample container 801.

Advantages of Controllable Flow

The ability to control the linear velocity of particles in a stream for a field focused system while maintaining high particle analysis rates in relatively large dimension channels enables improved analysis of particles and practical analysis of particles in ways that were previously not feasible.

By using lower linear velocities than conventionally used in flow cytometry and allowing each particle to spend longer times in the interrogation light, for example a laser, one can achieve the extremely high sensitivity seen in slow flow hydrodynamic systems without the drawbacks of clogging and low throughput. In addition, the utility of markers that are not typically used in cytometry because of fast transit times can be greatly increased. Among these markers are luminescence probes such as lanthanides and absorptive dyes such as cytological stains and trypan blue. Imaging of particles is also much more easily achieved by using slow flow or even stopped flow without resorting to specialized tracking technology like that used in imaging hydrodynamically focused particles (Amnis, Seattle, Wash.).

While nearly all labels currently used in cytometry would benefit from lower laser power to reduce photobleaching and non-radiative states and the longer integration of signal afforded by longer transit times, some that will benefit more than others are discussed herein. These include fluorophores/luminophores that have long lifetimes and or low quantum yields/extinction coefficients. Most chemi bioluminescent species also benefit tremendously from longer transit times as their energy is given off on time scales much greater than those used in conventional cytometry analysis. Long lifetime labels are for example labels with life times greater than about 10 ns. For example: labels with life time between about 10 ηs to about 1 μs, labels with life times between about 1 μs to about 10 μs labels with life times between about 10 μs to about 100 μs, or labels with life times between about 100 μs to about 1 ms and above.

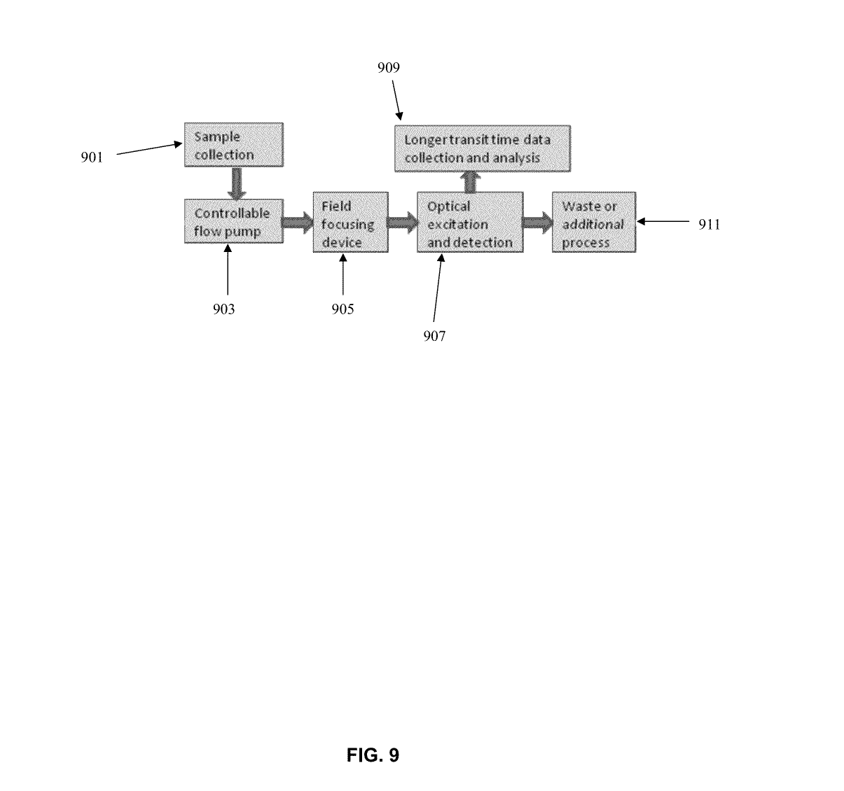

One aspect of the various embodiments disclosed herein provides for controllable linear velocity ranging from 0 m/s to 10 m/s without compromising core diameter and particle concentration. In a preferred embodiment the linear velocity is in the range of about 0 m/s to about 0.3 m/s. In a more preferred embodiment the linear velocity is in the range of about 0.3 m/s to about 3 m/s. In a more preferred embodiment the range is between about 3 m/s to about 10 m/s. Referring now to FIG. 9, a flow diagram illustrating various embodiments is illustrated. A field based means 905 focuses particles into a line or plane, preferably acoustically. The particles are transited through the system preferably by a pumping system 903 that can be adjusted to the desired flow rate for the desired linear velocities. A means for optical excitation 907 of the particles and a means for collection and analysis 909 of fluorescent/luminescent light given off by the sample comprising the particles. Average linear fluid velocity is given by the flow rate divided by the cross sectional area but particles will generally travel at nearly the same speed as the fluid lamina they are in. Particles focused to the center of a channel for most channel geometries used would travel about twice the average velocity. Preferably, the system provides possible pulsed or modulated excitation at slower rates, data systems to accommodate longer transit times and slower pulse rates and reduced waste that can readily be run again or transferred to another process 911 without concentration.

Slowing Linear Velocities—Single-Line Focused System

Various embodiments comprise methods to improve signal by increasing the number of photons given off by a fluorescent/luminescent label by illuminating it for a longer time period with a continuous light source and particles with a linear velocity of 0.3 m/s, this number increases over the prior art by about 10 fold. At this velocity and assuming an average of 100 microns distance between particle centers, about 3000 particles per second can be analyzed. It is the combined ability to focus particles and concentrate them that allows these long transit times for high particle analysis rates. If the velocity is further decreased to 0.03 m/s, 100 fold more photons would be given off and 300 particles per second could be analyzed.

In another example, semiconductor nanocrystals also referred to as quantum dots are highly resistant to photobleaching, so the gains predicted in the above example might not be so dramatic for other fluorophores that are prone to photobleach. All fluorophores however, are limited in continuous excitation by a power threshold that achieves “photon saturation” by exciting a maximum number of the fluorophores at any given time. Any more excitation photons will not produce any more fluorescence and will in fact decrease fluorescence by increasing photobleaching or exciting to non-radiative states. Often, one must balance excitation power with photobleaching rate and non-radiative state excitation such that the most fluorescence is emitted for a given transit time. In short, longer transit times will yield more photons for a given excitation power, but reducing light source power can further yield more photons per fluorophore during the analysis time. This is particularly important for high sensitivity applications in which very few labels may be bound to the target. Lower laser power also reduces fluorescent background, which can further increase sensitivity and resolution of particle populations.

Slower linear velocities can increase the signal from any label, but it also makes it practical to use dimmer labels and long lifetime labels that are not commonly used for lack of photon yield in short transit times. Lanthanide chelates, for instance, have very long Stokes shifts and very narrow emission wavelengths so they can be highly specific labels but they will emit relatively few photons over a short transit period. Nanoparticles using europium, for instance, may have lifetimes of about 0.5 milliseconds. In a field focused system, transit times can be slowed to milliseconds or more allowing several cycles of excitation and emission to be monitored. Downstream optics are not required.

Pulsed or Modulated Excitation

According to various embodiments an excitation source is pulsed or modulated. Many commercial light sources are available to do this by affecting the light source itself and using digital or analog control. Methods such as chopper wheels and acoustoptic modules can also modulate the interrogation light source externally. For some applications it is also desirable to sync the detectors with the light source in time such that events can be correlated to excitation peaks or valleys. Correlating a Rayleigh scatter detector that detects light scattered from passing particles with the fluorescence detector(s) in time is one preferred method. Lock-in amplification can be used to help eliminate electronic noise at frequencies other than the modulation frequency but averaging to eliminate noise can be accomplished digitally if the data is collected in a digital format.

Using pulses with relatively long rest times that allow relaxation from triplet states can increase the overall fluorescence yield of fluorophores vs. equivalent power strong continuous wave excitation. This is again of particular importance to high sensitivity applications where only a few fluorophores are present. For a slow transit system, as in the various embodiments disclosed herein, particularly a field focused system, 67 pulses with microsecond timing can be monitored for a 0.3 m/s linear velocity for probes such as perCP where the triplet state is estimated to be about 7 microseconds. Pulsed or modulated light sources have the additional advantage of allowing phased locked amplification or averaging of time correlated data, either of which will reduce electronic noise.

Photobleaching of Undesired Fluorescence

One can take advantage of labels that have a high resistance to photobleaching by strongly illuminating a cell or particle having undesired fluorescence (often cellular autofluorescence) or the medium the particle is in, e.g. serum. The use of long transit times to accomplish this allows more photobleaching for a given excitation power. While in-flow upstream photobleaching is effective in a long transit time system, it can also be done in the various embodiments disclosed herein with a single light source in a long transit time system by examining the signal as the cell passes. The fluorescence of the less resistant species will decrease more quickly than that of the resistant specific label. This decrease not only increases specific signal to noise but the rate of decay can also be used to separate the non-specific signal from the specific signal by allowing a quantitative subtraction of the autofluorescence present. Quantum dots are an example of a good label for this purpose not only because of their high photobleaching resistance but because of their long Stokes shift. The Stokes shift can move the signal out of the primary cellular auto-fluorescence peak which improves signal to noise already but it also opens that spectral wavelength for use of an additional detector to monitor the auto-fluorescence. This by itself has been used to subtract cellular auto-fluorescence but the technique could be made more effective by also monitoring the decay rate. Decay rate can also be used to compensate bleed-through for different channels (colors) of fluorescence. If, for example a particle is labeled with both fluorescein and phycoerythrin and is excited with 488 nm light, the relative decay in the green and red channels can be used as a quantitative measure of how much fluorescein fluorescence is picked up by the red channel. This method improves compensation accuracy and eliminates the need for running compensation controls.

New Useful Probes

Lanthanide chelates, especially those using europium and terbium have dominated the time resolved probe market. These complexes are generally excited by wavelengths shorter than 400 nm but developments in these probes, e.g. Eu(tta)3DEADIT (Borisov and Klimant), have resulted in complexes that can be very efficiently excited by 405 nm light. This is significant because the low cost, high quality 405 nm diode lasers developed in the entertainment industry promise to lower the cost of violet excitation. Many other metal ligand complexes excited at a variety of wavelengths have high potential for use in longer transit time cytometers. The list can be further increased by including luminescence resonance energy transfer (LRET) probes which general combine a long lifetime fluorophore like a metal ligand complex with a shorter lived dye. Lackowiz, Piszczek and Kang (2001) found that in such complexes it was possible to achieve a high quantum yield fluorophore by combining a low quantum yield metal ligand complex donor with a high quantum yield acceptor by combining a ruthenium ligand complex with short lifetime dyes.

These types of tandem probes are particularly useful in a long transit time cytometer because the long lifetime of the donor and the short lifetime of the acceptor combine to give a medium lifetime probe that would have too long a lifetime for a conventional cytometer but a short enough lifetime to increase throughput in a long transit time cytometer. For example, the ultra long lifetime of a terbium complex in a DELFIA™ assaying format has a lifetime of 1045 microseconds as compared with a Terbium fluorescein complex in a LanthaScreen™ assaying which has a lifetime of 160 microseconds. Lifetime can also be manipulated with changes to the metal chelating ligands (Castellano et at. 2000). With numerous possible metal ligand complex as donors and no limit to the number of acceptors many useful probes can be developed on the basis of spectral and lifetime properties. The multiplex idea can be carried even further using probes having several differing lifetimes e.g. short, medium and long that can be resolved individually by lifetime. A great advantage for the lifetime multi-plexing scheme is that the same detectors can be used for overlapping colors, e.g. a fluorescein/terbium complex can be used in conjunction with plain fluorescein.

Qdots® although shorter lived than luminescent probes, have lifetimes that are long enough (˜10-100 ns) to be well separated from most conventional fluorophores and short enough to be used in conventional cytometers but the practical use of lifetimes on this scale has been limited. Developments in high speed detectors, lasers and electronics make this more practical.

Other luminescent materials such as phosphors and up-converting phosphors have not achieved success in bioassaying, largely due to their large size. These materials might be very useful however in multiplex beadsets for cytometry. Their emissions can be distinguished using time resolved techniques and the up-converting phosphors can be excited using long wavelength lasers that would not excite most fluorophores used in assaying.

Secondary Reagents

Secondary reagents using ligands such as biotin, streptavidin, secondary antibodies and protein A and G will be of particular utility in inexpensive cytometers and long transit time cytometers. For instruments taking advantage of violet diodes, availability of violet excited dyes conjugated to the antibodies or other ligands necessary for assaying may be in short supply, so violet excited secondary conjugates will be very useful, e.g. Pacific Blue® or Orange® conjugated to streptavidin/biotin or protein A/G or anti-species specific or probe specific (fluorescein, PE, APC) secondary antibodies can all be used to increase the utility of an instrument with fewer lasers than are typically necessary to excite probes of choice. If, for example, assaying requires antibodies that are only available as unconjugated or conjugated to Fluorescein or PE or biotin needed to be performed in a violet only or a violet and red instrument, protein A or G or species specific secondary reagents can be used for unconjugated antibodies, labeled streptavidin for the biotin antibodies and anti-fluorescein or PE for the dyed antibodies. Qdots® are also excellent examples of violet excited labels that are typically used in a secondary format, usually streptavidin conjugates. Their broad excitation spectrum makes them particularly suited for a single violet laser system or a violet/red laser system where inter beam compensation can be minimized.

With acoustic washing, secondary labeling can be accomplished very quickly and easily by primary labeling, acoustic washing, secondary labeling and a second acoustic wash. An automated system or semi automated system to do this will reduce not only assaying time but operator error.

Secondary long-lifetime labels are particularly suited to a long transit time cytometer with modulated or pulsed excitation as they allow adding the lifetime parameter for analysis using commonly available antibodies/ligands.

Other Long Lifetime Methods

For bio/chemi/electro luminescence one can use a pulsed/modulated system that analyzes the level of luminescence in between pulses and subtracted this from the fluorescence for short-lived labels. This luminescence might be measured using reagents internal to the cell or can be membrane bound enzyme labels that interact with substrate added to the sample. Acoustic washing just prior to analysis could ensure that luminescence from the medium could be associated with the proper cell. Monitoring enzyme cleaved substrates in a more conventional manner after sorting is another possibility for drug discovery assaying but it can also be applied to low level marker assaying that require enzyme amplification for detection.

Various embodiments disclosed herein comprise methods for measuring chemi, bio or electro luminescence in an acoustic particle analyzer. In these methods, particles capable of producing a chemi, bio or electro luminescence are moved through a channel and are acoustically focused using acoustic radiation pressure. The particles are then passed through a zone for collection of luminescence and collect light from the particle produced from chemi, bio or electroluminescence. In these embodiments, the particles are preferably focused with a radial acoustic field. Luminescence is preferably collected between excitation pulses from a light source.

Time-Resolved Fluorescence/Luminescence

Another embodiment of the present disclosure provides a method for circumventing background fluorescence using probes that continue to emit light for some time after the background fluorescence has substantially decayed. The advantages of slower linear particle velocity make the technique much more attractive. This method uses a modulated or pulsed laser as above but light is also collected and correlated in time to the excitation valleys where there is little or no excitation light. The longer time intervals that are not implemented in conventional flow cytometry cost much less with lower cost lasers and electronics, but their primary advantages lie in the ability of maximizing fluorophore output and to use very long lifetime probes such as lanthanide chelates and lanthanide energy transfer probes. Pulsing at the very slow (for flow cytometry) rate of a thousand times per second with a 10 microsecond pulse, would for a transit time of 10 milliseconds for example, allow 10 cycles of excitation and luminescence collection in which virtually all of the luminescence decay of a europium chelate could be monitored. This pulse rate with a conventional cytometer transit time would allow >90% of the particles to pass without ever being hit by the laser. If the pulse rate were increased to 100 kilohertz with a 1 microsecond, pulse there would still be nearly 9 microseconds in which to monitor the lanthanide luminescence as most fluorophores have 1-2 nanosecond lifetimes and most autofluorescence decays within 10 nanoseconds. For some lanthanides specificity can further be increased by monitoring fluorescence of different emission peaks.

The most commonly used lanthanides, terbium and europium, are prime examples. For the Seradyn europium particles for example, their two primary emission peaks are at 591 and 613 nm. The ratio of these peaks (˜13) is a highly definitive signature of this label. The peaks can be readily distinguished from each other as their bandwidth is so narrow (90% bandwidth for the 613 nm peak at 25 nm) an additional degree of specificity could also be achieved from tracking the kinetics of this emission as the 591 nm peak has a shorter lifetime than the 613 nm peak and the rate of change of the ratio would be extremely specific. In a preferred embodiment increasing luminescence of the label is monitored during subsaturation excitation pulses to monitor specificity. If the labels are not excited to saturation and if the dead time between the pulse is less than the fluorescence decay, each successive excitation cycle would increasingly excite more labels before the decay of other excited labels is complete. This gives an increasing trend in signal that is specific to the lifetime of the probe. With this method, it is not specifically the phase or lifetime that is being measured but the increase in the phase shifted emission. In principle, this can be done with other labels such as quantum dots but as their decay times are much shorter (10-100 ns), a much quicker pulse or modulation rate is required (˜10-100 MHz). Careful attention must also be paid to the excitation intensity and formation of triplet states such that the specificity advantage is not lost by collection of fewer photons.

One of the primary sources of unwanted signals in flow cytometry and label based techniques in general is the specific fluorescence of unbound or non-specifically bound labels. For flow cytometry, squeezing the sample core size to a very small dimension and optical spatial filtering alleviate the problem of unbound labels to some degree, but ultrasensitive applications often require prewashing of the analyte species from the unbound labels. This is problematic if the labels are not of extremely high affinity as they may tend to dissociate from their targets once washing disturbs the binding equilibrium or kinetics.

Field based in-line particle washing is used to solve this problem. Laminar flow washing relies on the fact that only particles affected strongly enough by the field to move across the laminar boundary will enter the clean fluid. This generally leaves most of the labels behind. According to one embodiment of the present disclosure, the fluidics are constructed such that substantially clean fluid reaches the collection or analysis region. Some clean fluid is discarded with the waste in order to account for diffusion across the laminar boundary and insure high purity.

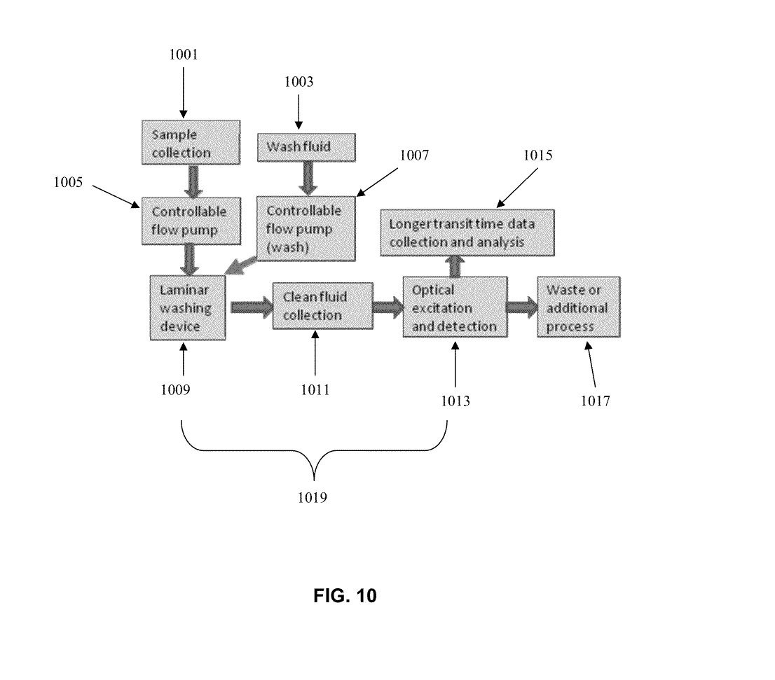

Using in-line field based washing allows for very small time intervals between the alteration of binding kinetics and the analysis. By placing the wash step very close to the analysis point, washing can be achieved readily in fractions of a second. Even for relatively low affinity binding reactions, background reduced analysis can be done before significant dissociation occurs. This capability is of high importance for many applications where sensitivity is important, but binding affinity is relatively low. Many monoclonal antibodies, synthesized ligands and drug candidates fall into this category. FIG. 10 illustrates the flow chart in FIG. 9 modified to include in-line laminar washing. FIG. 10 comprises an additional pumping system 1007 used to introduce the wash fluid and fluidics which are modified to extract the cleaned particles after washing. Laminar wash devices can also be installed in series to increase purity or to process particles in different media, see example FIG. 8.

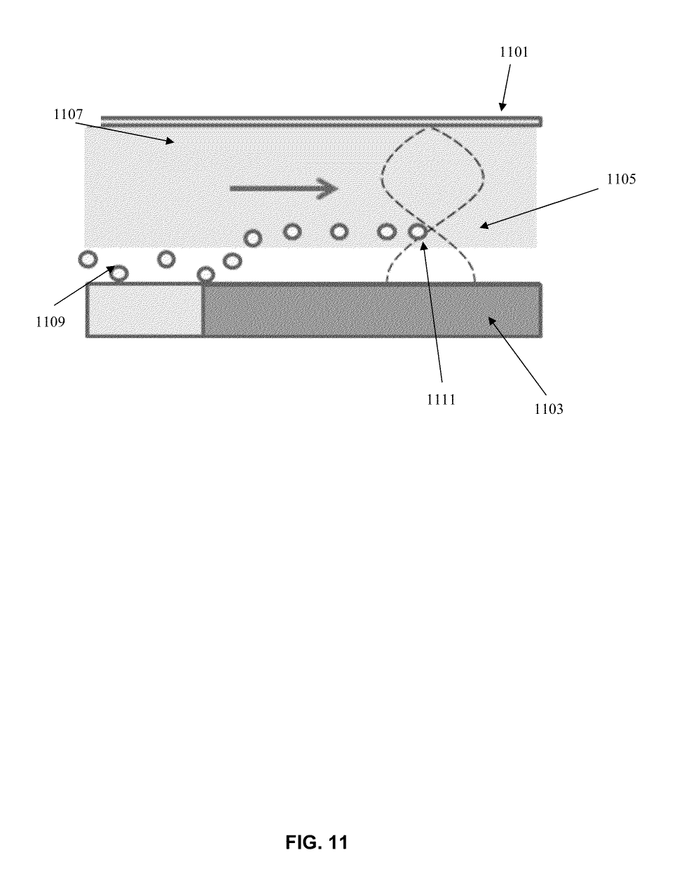

As illustrated in FIG. 10, an acoustic focusing device 1019 is modified with wash stream 1007 into which target particles can be focused. Washed particles can be analyzed within fractions of a second of being washed. For the planar device in FIG. 11, focusing is only in one dimension but this dimension can be stretched out over a large area to increase flow rates.

FIG. 11A is a planar acoustic flow cell comprising laminar wash fluid 1107. Sample containing particles 1109 is introduced into flow cell 1101. An acoustic wave is introduced 1105. Particles are acoustically focused based on acoustic contrast. Channel tuning is dictated by height rather than width. This allows high width to high aspect ratio channels with higher throughput. Different standing waves can be used in accordance with FIG. 11A.

FIG. 11B illustrates a planar acoustic flow cell wherein the acoustic node is located outside flow cell 1101. In this embodiment, particles 1109 are acoustically focused to the top of the flow cell where acoustic wave 1105 is introduced.

Multi-Color Analysis

Most of multiplexing in flow falls into two distinct categories. The first is often referred to as multicolor analysis in which several markers on or in cells are examined simultaneously in order to extract as much information as possible from the cells being analyzed. Probes of different spectral wavelengths are chosen such that there is maximal excitation overlap and minimal emission overlap and usually the markers that are known to bind the fewest labels will be given the brightest probes in hopes that all markers can be resolved. Tandem probes are useful such that one or two lasers could be used to excite many fluorophores with different emission spectra. In practice there is considerable spectral overlap between probes and a great deal of effort is expended on subtracting the signal contribution of these overlaps such that each individual probe is accurately quantified.

One embodiment of the present disclosure provides a system and method to produce greater signal to noise resolution of the specific signals and improved methods for isolating the different probes. First, the use of longer lifetime probes such as the narrow emission of quantum dots and of lanthanides can be better exploited to extract individual signals with less compensation. By using pulsed or modulated lasers, the fluorescence lifetime of the probes can be monitored to determine the individual contributions of different dyes. Even if there is fluorescence contributed to detection channels monitoring shorter lifetime probes, this fluorescence can be subtracted based on the quantity of time resolved fluorescence detected. In an embodiment, the increased signal generated for the longer transit times by using narrower bandwidth filters is utilized. These filters collect less light but do a better job of separating fluorescence signals from different probes. The band width can be made narrower than in conventional systems because there is more signal to spare. The narrow bandwidth approach collects the entire spectrum with a prism or grating and multi-element detectors. In this case the resolution of the spectrum dictates how much bandwidth per element is collected. Longer transit times make spectral collection much more practical.

Multiplexed Assaying

The second form of multiplexing in flow is the use of multiple bead populations to encode simultaneous assaying such that each can be distinguished from each other. Specific chemistry is placed on each population and then the populations are mixed in a single reaction vessel and are then processed in flow. The distinctive properties of each population such as size and or fluorescence color and or fluorescence intensity are then detected to distinguish the beads from each other. The assay on each bead must be distinguished from the bead's intrinsic properties and this is typically done by using a different color fluorescence for the assaying itself. These multiplexed soluble arrays may be used in diverse applications in accordance with the present disclosure including but not limited to immunoassaying, genetic assaying, and drug discovery assaying.

One type of soluble bead array uses two fluorescent dyes that are doped into the beads in varying concentrations to produce populations with distinct fluorescent color ratios. Between two colors and ten intensities an array of 100 distinct beads is made. It is sometimes difficult to resolve all of the beads due to variance in the beads and the detection systems. Longer transit times and the associated higher signal increase sensitivity and resolution. This means that for any color coded multiplex system more intensity levels can be resolved and lower concentrations of dyes or other labels need be used. Quantum dots are excellent for creation of soluble arrays owing to their narrow spectral emission and their ability to all be excited by a single laser. According to one embodiment of the present disclosure a system and method provides for longer transit times of particles having quantum dots. Beads may be made with much fewer quantum dots. This makes them less expensive with a lower background interference for assaying. It also makes for more resolvable intensity levels such that greater numbers of distinct beads can be made. A set using 4 colors of Q-dots and 5 intensities for example yields a set of 625 distinct beads. The same 4 colors and 10 intensities yields a set of 10,000.

A difficult problem in multiplexed arrays is distinguishing the coding fluorescence from assaying fluorescence. This is a particular problem when high sensitivity is required from a high intensity coded microsphere. The methods detailed above for multi-color analysis can all be used to make this easier. In particular, long fluorescence lifetime probes, including but not limited to lanthanide labels with quantum dot arrays and time-resolved fluorescence, may be used. Lanthanide labels are mostly excited by ultra violet light (europium 360 nm max absorption). This allows them to be used in conjunction with quantum dots and a single excitation source (e.g. 375 nm diode laser, pulsed or modulated for life-time applications). The lanthanides' very narrow spectral emission can also be effectively used for lanthanide based microsphere arrays.

By choosing dyes with different fluorescence lifetimes, arrays can also be made based on lifetime alone. If for example both a conventional short lifetime UV excited dye and a long lifetime lanthanide dye were impregnated at discrete concentrations into bead sets, both dyes can be excited by a pulsed/modulated UV source and the rate of emission decay would be specific for each discrete concentration. Neither dye would be excited by longer wavelength lasers, particularly the blue, green and red lasers most common in cytometry. This allows for monitoring assaying across a wide range of probe colors. In addition, this lifetime decay method can be implemented with a single photodetector and a simple filter to block scattered light at the UV excitation wavelength. The lifetime method can also be combined with other multiplexing methods such as particle size and or multicolor multiplexing to increase array size.

In another embodiment of the present disclosure, fluorescence is separated from luminescence by collecting light as the particle travels in and out of a continuous light source. Fluorescence is collected while the particle is illuminated and luminescence just after the particle has left the illumination. This can be done with properly spaced collection optics or over the entire space using a multi-element detector such as multi-anode PMT or a charge coupled device (CCD).

In another embodiment of the present disclosure, coupled with imaging optics, the CCD above, or another imaging detector can also perform time resolved imaging on focused particles. This is not done in flow cytometry. In fact any imaging technique that requires longer excitation and or emission exposure than is afforded in conventional hydrodynamic focusing is possible in a field focused system.

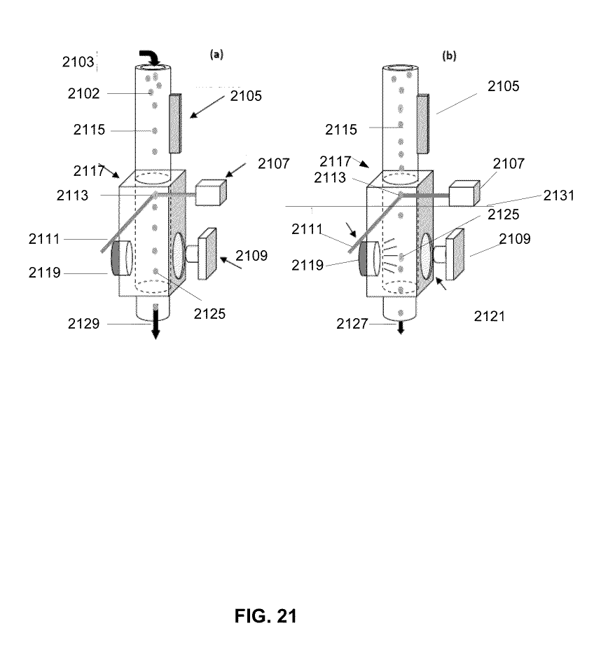

Referring now to FIG. 21(a) which illustrates an embodiment of the present disclosure, sample 2103 containing particles 2102 is introduced in the system. Line drive 2105 induces an acoustic wave and particles 2102 are acoustically focused based upon their acoustic contrast 2115.

Optics cell 2117 receives particles and interrogates each particle at an interrogation site where interrogation source 2111 impinges electromagnetic radiation upon particle. An optical signal 2113 from each particle and/or sample is collected by 2107.

The signal is analyzed and based upon user determined criteria and optical signal from a particle, a selected particle or group of particles is imaged by an imager 2109. The particle may receive an illuminating light from a light source 2119 for imaging. In FIG. 21(a), no image is acquired and the flow rate 2129 remains unchanged.

The flow rate can be altered once a particle meeting a user defined criteria is detected at 2107. The particle at 2125 in FIG. 21(b) is moving slower than particle 2125 in FIG. 21(a) because the flow rate is decreased to acquire an image in FIG. 21(b). The flow rate is decreased once a particle having the user defined criteria is detected at 2107. An image of the particle is acquired by imager 2109 and the image may be illuminated by an illumination source 2119. Once the image is acquired the flow rate remains decreased 2107. Alternatively, the flow rate is increased for improved particle throughput through the system.

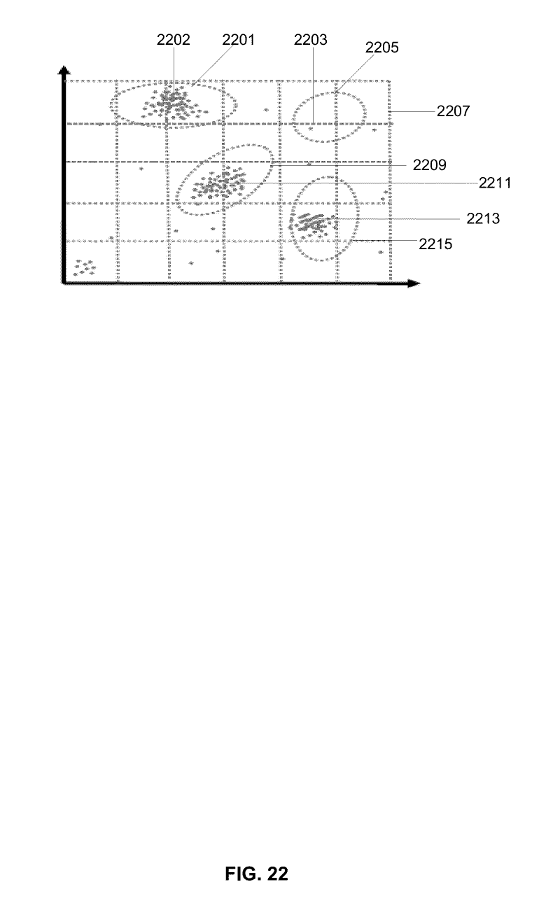

Referring now to FIG. 22, a bivariant plot of particles analyzed in a system as shown FIG. 21 is provided according to one embodiment of the present disclosure. Each particle within a group of particles 2202 are similar as to Parameter 1 and Parameter 2. Parameter 1 can be, for example, forward scatter, side scatter or fluorescence. Parameter 2 can be, for example, forward scatter, side scatter or fluorescence. The user defined threshold 2201 indentifies particles that meet a threshold for imaging. A particle having a value for Parameter 1 and for Parameter 2 that is greater or lesser than the threshold defined by the user triggers the imager and is imaged. If the particle meets the user defined criteria then the flow of the stream carrying the particles is reduced to a rate that allows the imager to capture an in-focus image of the particle or particles as the particle transits past the imager 2109 of FIG. 21.

Other detection thresholds 2205, 2209 and 2215 can be established for particles having similar Parameter 1 and/or Parameter 2 values.

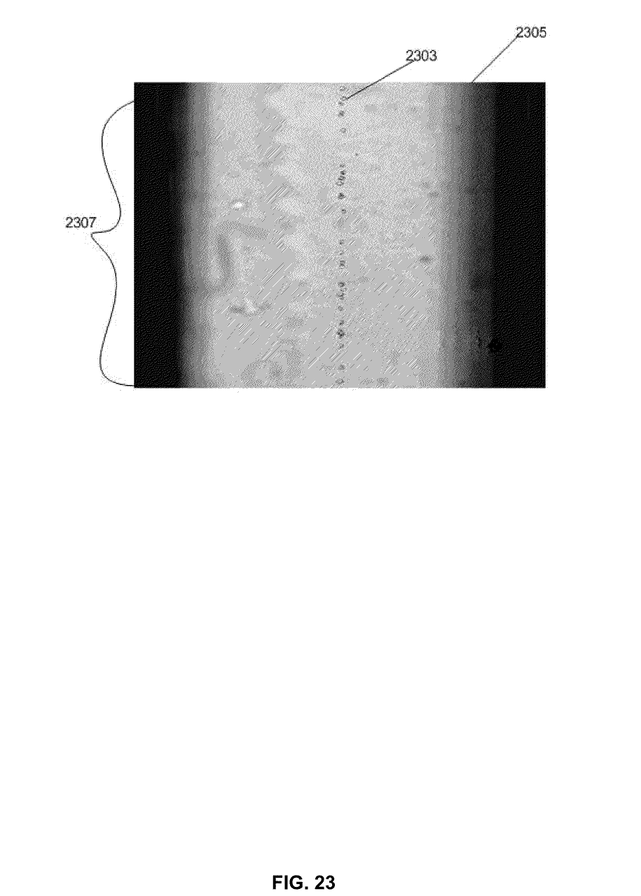

Referring now to FIG. 23, is a photograph of blood cells 2303 are captured for a stream 2305 that is acoustically reoriented. The stream in the optics cell 2307 is slowed so the imager (not shown) can capture particles 2303 in focus. To create the image, a line-driven capillary of inner diameter 410 μm is truncated with an optical cell. The optical cell is a borosilicate glass cube with an interior circular cylindrical channel the same diameter as the inner diameter of the line-driven capillary. The frequency of excitation is approximately 2.1 MHz and the power consumption of the acoustic device is 125 milliWatts. The cells are lined up single file coincident with the axis of the capillary. In this image, flow is nearly static for imaging purposes, but our recent engineering advances in constructing line-driven capillary prototypes have proven fine focusing of 5 μm latex particles and blood cells at volumetric flow rates exceeding 5 mL/minute.

In a preferred embodiment of the present disclosure, a line-driven capillary is attached to a square cross-section quartz optics cell. The inner cavity of the optical cell is circular in cross section and has the same inner diameter as the line-driven capillary to extend the resonance condition of the fluid column thereby extending the acoustic focusing force into the optical cell. In operation, the particles are aligned along the axis of the capillary by the acoustic force and fluid flow transports them through the system. The particles first enter the analysis stage where an incident laser beam excites the particle and attached fluorescent markers. When a target of interest is identified by its scatter and fluorescence signature (user defined), the control system decelerates the flow velocity to a value appropriate for the required imaging resolution. A flash LED (wideband or UV) then illuminates the particle to capture the image. Once the image is captured, the system re-accelerates the flow in the original direction and analysis continues until another particle of interest is encountered.

To achieve the high analysis rates expected from a traditional flow cytometer analyzer, the embodiment will not capture an image of every particle analyzed in the system. Rather, the user will construct a sampling matrix of particles from gated subpopulations to define a set of particle images to be captured based upon their scatter and fluorescence signatures. With this hybrid approach, high particle analysis rates are achievable (in excess of 2000/s to search through large populations of cells while capturing only a representative set of high content images that are correlated with traditional flow cytometry parameters. Images will capture cellular morphology, orientation, and internal structure (e.g. position and number of nuclei) that will be available to the researcher to correlate with localized data distributions generated by the analyzer. The ability to control flow rate while maintaining particle focus along the axis of the flow stream in the acoustic system is the key component necessary for the selective particle imaging after sample analysis.

Fast coaxial flow streams are not required as with conventional hydrodynamically sheath-focused systems. This alleviates the need for differential pressure or flow based delivery systems reducing total system cost. One of the unique capabilities of acoustically driven flow cells is the ability to select the sample delivery rate. By not accelerating the particles with the coaxial sheath flow, particle transit times through the laser interrogation region of a flow cytometer are ˜20-100 times longer than conventional hydrodynamically focused systems. In comparison, higher sensitivity optical measurements can be made while retaining similar particle analysis rates. This enables the use of inexpensive optical components to lower system costs. Additionally, acoustically reoriented sample streams can be operated at slow fluid velocities or even stopped and reversed without degrading alignment of the particle stream within the flow chamber allowing rare targets to be repeatedly analyzed or even imaged.

Imaging is performed commercially in flow cytometry using imaging optic by fluid imaging. The fluid imaging uses deep focus optics without hydrodynamic focusing to take pictures of cells or particles, or even organisms as they pass through a rectangular imaging cell. The method has adjustable flow but it is limited to taking pictures of particles in focus so many are missed and magnification power and resolution is limited. One system uses hydrodynamic focusing coupled with electronic CCD panning technology that can track the flowing cells. The system is designed to keep cells or particles from being blurred. Imaging rates for this system are relatively slow (up to 300 cells/sec) but slower flow and the tracking technology allow long integration times that keep sensitivity high and allow good spatial resolution (up to 0.5 microns). Unfortunately, this technology is very expensive and is limited by the hydrodynamic focus.

Acoustic cytometry of the present disclosure, in which hydrodynamic focusing of target cells or particles is replaced or partly replaced by acoustic radiation pressure, adjusts the linear velocity of cells or particles transiting an interrogation laser while maintaining tight particle focus. Therefore, light from photoactive probes can be collected for much longer times than are normally possible in hydrodynamically focused cytometers without loss of precision from poor particle focus. Flow in an acoustic cytometer can even be stopped or reversed, allowing very long observation or imaging for resolution of spatial information.

One advantage that field focused systems have over hydrodynamic focused systems, is again the control of linear velocity while maintaining particle focus and high analysis rates. Greater transit times can be used all the way up to stopped flow. Pulsed flow is a viable option in field focused systems in which fluid delivery is triggered by upstream detectors such that cells or particles are stopped in the imaging region and flow is maximized when no particles are present. This method increases throughput while maximizing exposure times.

Another embodiment of the present disclosure provides a method to increase throughput in field focused systems with a planar focusing system such as that shown in FIG. 11. For this system, many particles can be imaged simultaneously. By using a wider field of view, some spatial resolution may be lost, but many applications do not require diffraction limited resolution. Controllable velocity can make this technique extremely sensitive and also easier to implement. Pulsed flow can also be used by taking images of particles/cells in batches: take a picture, then flush out the already imaged cells while replacing them with a new batch of cells.

The statistical power of the cytometer of the present disclosure and the spatial resolving power of imaging are combined when additionally imaging particles. This combination is very significant for numerous applications where cell morphology and or localization of markers are important. Fluorescence in-situ hybridization (FISH), cancer screening, intracellular and membrane protein/drug localization and co-localization are a few of the analyses that could benefit tremendously. Other applications such as industrial process monitoring or monitoring of environmental samples can also benefit.

Another application for imaging in flow is the use of spatially barcoded particles for multiplexed assaying. The microfluid method ensures that the particles align properly by using very small channel dimensions. The acoustic focusing preferentially orients such particles in the field making it possible to use much larger channels that are not prone to clogging.

Methods for Monitoring Cell/Particle Kinetics in Field Focused Particle/Cell Analysis Systems

Another embodiment of the present disclosure provides for cell monitoring or particle reaction kinetics by the imaging methods above but these kinetics can also be monitored without imaging in field focused systems with long transit times. The transit times can be adjusted to monitor whatever process is of interest. The method lends itself very well to techniques that use light activated species such as caged fluorophors or ions, photoactivated GFP or photoactivated ATP or GTP. Such techniques are absent in flow cytometry due to the need for longer analysis times.

Kinetics Quantification

The quantification of kinetic parameters such as antibody binding constants and enzyme substrate cleavage rates can be quantified using in-line acoustic washing and analysis. By placing reactant of a known concentration in a laminar stream and acoustically transferring the reactive particles into the stream such that the time of exposure to each other is known, one can determine kinetic parameters using data collected from the beads in a cytometer. For example, a new antibody can be tested by switching antigen coated beads stained with fluorescent antibody with known constants into a stream containing a known concentration of the new antibody. The fluorescence of the beads relative to controls indicate the new antibody's ability to displace the known antibody. The time of interaction can be varied with flow rate. If the constants to be measured are longer lived, starting the reaction by prediluting with reagent and measuring fluorescence over the course of analyzing the whole sample is another alternative.

Microsphere beads are used for a myriad of applications in sample preparation and purification. Among the most common are nucleic acid separation, protein fractionation and affinity purification, cell isolation and cell expansion. Beads are generally separated from sample media by centrifugation or magnetic means. The microsphere beads can also be separated by acoustic means and are typically denser and less compressible than most biological materials. For these beads, acoustic washing with a fluid stream of high enough acoustic contrast to largely exclude sample materials while allowing central focus of the beads allows execution of protocols otherwise employing magnetic means and centrifugation. For many protocols, this allows cheaper non-magnetic beads to be used. It also provides for automation of steps that are typically carried out in sample tubes.

Magnetic and acoustic forces can also be used in tandem, allowing ternary separations of magnetic and acoustic beads or magnetically and acoustically labeled cells. Of course, magnetic beads can be used in a conventional manner and then be further processed or analyzed using acoustic sample prep and or an acoustic cytometer. This combination can be quite powerful as magnetic forces generally excel at quickly and conveniently separating targets from concentrated samples while acoustic cytometry excels at quickly processing dilute samples. Multiplexed magnetic bead arrays are a prime example of this where the convenience of tube preparation is combined with the power of acoustic cytometry analysis.

### Example

### Negative Contrast Particles Nucleic Acid Isolation

Negative contrast particles modified for nucleic acid capture are incubated with a lysed sample and flowed through the acoustic separator where they are forced to the outside walls. They are then washed with the acoustic field on and resuspended with the field off. Washing in this way can be repeated as many times as is desired. If the particles are of low enough acoustic contrast, they can also be washed with isopropanol and ethanol as required. Nucleic acid elution can be performed with the appropriate buffer and particles can be removed by simply turning on the acoustic field. Alternatively, further reagents can be added for nucleic acid amplification directly in the chamber, as the required thermal control if implemented.

### Example

### Microbe Isolation from Blood

The low level of microbes found in blood during sepsis poses many challenges to sample prep for concentration and isolation of the pathogen. Acoustic washing can be implemented in ways that solve many of these challenges. By flowing clean media down the center and flowing contaminated sample around the clean central core, smaller microbes can be effectively excluded from collection in the central core. The central collector removes blood cells and the outer collector, which at this point contains mostly platelets and the contaminating microbes, proceeds to a second separation in which a core dense enough to exclude platelets but not most microbes is used to collect and concentrate microbes. The collected sample can then be concentrated further or be sent to analysis and/or be cultured.

Electroporation

The possibility of washing cells into a medium for lysis or permeating membranes is possible using acoustic washing. This idea can also be applied to electroporation whereby cells are acoustically focused into a reagent loaded stream and are flowed through an electric field that permeates the cell membrane and allows reagent entry into the cell. The field can also be used for other reactions like electroluminescence.

The single line focusing possible in a line-driven system allows precise control of the field parameters that each individual cell is exposed to. It also enables post electroporation analysis and sorting. For example, a host of different cell types can be processed and then sorted on the basis of cell surface markers and cultured and or analyzed for the effect as single populations. Electroporation can also be performed without acoustic washing but may be preferable for conservation of reagents or limiting cell exposure to reagents.

High Resolution Continuous Field Flow Fractionation Using Pre-Focusing

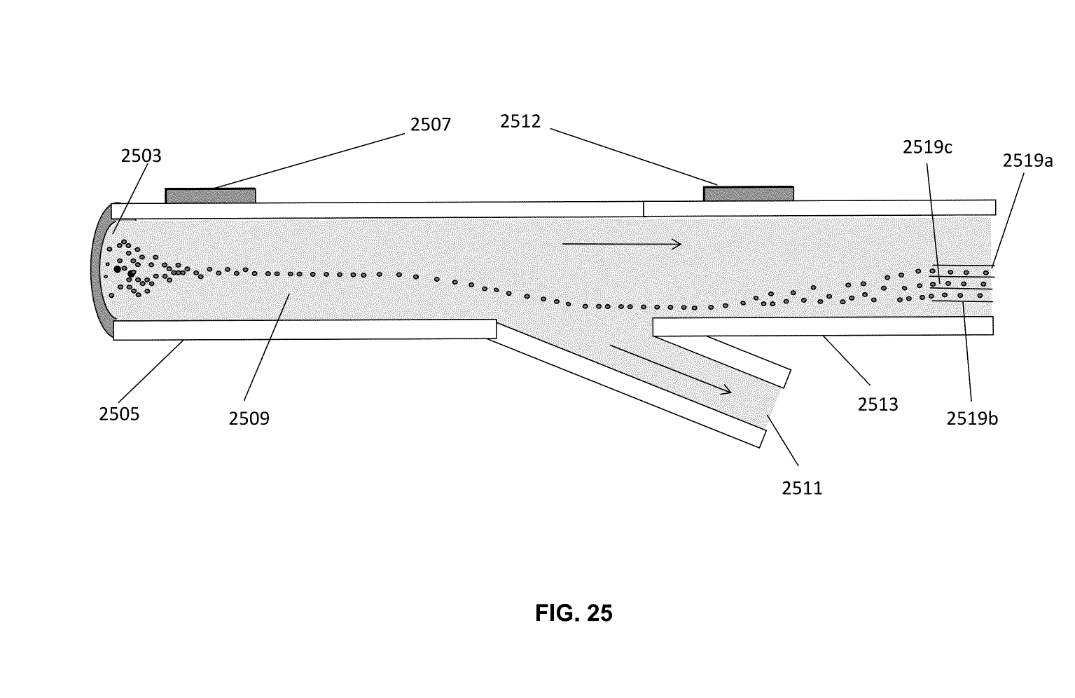

According to one embodiment of the present disclosure acoustic fields are used to separate particles based upon one or more characteristics of the particles such as size, acoustic properties or a combination of both. The uniformity of separation conditions with respect to each particle contributes to the precision of the separation efficiency. The ability to separate into discrete populations becomes compromised if a particle flows more slowly than another or if the particle is exposed to a different gradient field than another. Referring now to FIG. 25, focusing particles in acoustic capillaries to form a single file line is illustrated. The method can be used to provide uniform distance and acoustic field exposure during a particle separation by first passing particles in a sample into channel 2503. Particles within the sample are moved to first acoustic focuser 2505 which focuses them in single file line 2509 with first transducer 2507. An initial concentration can be adjusted to minimize aggregate formation in the acoustic field and insure minimal particle to particle interaction. The line of particles can subsequently be fed into acoustic separator 2513 equipped with transducer 2512 and multiple exit bins 2519a, 2519b, 2519c for separation and collection. The position of line of particles 2509 can be adjusted as it enters the separator portion of acoustic separator 2513 by drawing fluid away or otherwise removing fluid through, for example side channel 2511. Both flow rate and power can be adjusted to accomplish the desired separation. If several fractions are desired, the collector portion of the channel can be constructed in layers or bins to extract different fluid lamina. This layered construction can also aid in automated operation of the separations by reducing the need to adjust for parameters that might affect separations such as viscosity. If, for example, the viscosity changes in a particular separation due to for example, temperature change, a particular desired fraction might end up in a different bin than expected, but it can then simply be collected from that bin. Particles according to this embodiment can have a coefficient of variation improved by >40% or even >80%.

The system and method of the present disclosure holds particular utility for separation of microspheres that tend to be more poly-disperse as their manufactured size increases beyond about 3 microns. It is not uncommon for even relatively uniform size standards to have coefficients of variation above 10%. This corresponds to a standard deviation of 0.6 microns for a 6.0 micron particle. Resolution of the separation of populations within this variation can be very fine when the particles are well separated such that they do not interact with each other. Given that each particle has similar density and compressibility, the acoustic radiation force is proportional to the volume of the particle. Therefore, the force on a 6.2 micron particle is about 10% greater than on a 6.0 micron particle while the drag force is only about 3.2% greater. This means that in a uniform acoustic field, if the 6.0 micron particle is forced by the acoustic field to move 1 mm in a fluid, the 6.2 micron particle will move about 1.08 mm. This is more than enough movement to separate these particles into different bins. If by using this process standard deviation for collected fractions in the above example is reduced to 0.3 microns this represents a 50% improvement. Reducing it to 0.1 microns represents about an 83% improvement.

### Acoustic Cytometry Methods and Protocols:

According to exemplary embodiments of the present disclosure, a flow cytometer may be an acoustic flow cytometer configured to acoustically focus a sample in a flowing fluid using acoustic energy. For example, a flow cytometer may be an acoustic flow cytometer embodying one or more of the teachings of any one or more of U.S. Pat. No. 7,340,957, issued Mar. 11, 2008, U.S. Pat. Appl. Pub. No. 2009/0050573, published Feb. 26, 2009, U.S. Pat. Appl. Pub. No. 2009/0053686, published Feb. 26, 2009, U.S. Pat. Appl. Pub. No. 2009/0029870, published Jan. 29, 2009, U.S. Pat. Appl. Pub. No. 2009/0048805, published Feb. 19, 2009, U.S. Pat. Appl. Pub. No. 2009/0042239, published Feb. 12, 2009, U.S. Pat. Appl. Pub. No. 2009/0045107, published Feb. 19, 2009, U.S. Pat. Appl. Pub. No. 2009/0042310, published Feb. 12, 2009, U.S. Pat. Appl. Pub. No. 2009/0178716, published Jul. 16, 2009, U.S. Pat. Appl. Pub. No. 2008/0106736, published May 8, 2008, U.S. Pat. Appl. Pub. No. 2008/0245709, published Oct. 9, 2008, U.S. Pat. Appl. Pub. No. 2008/0245745, published Oct. 9, 2008, U.S. Pat. Appl. Pub. No. 2009/0162887, published Jun. 25, 2009, U.S. Pat. Appl. Pub. No. 2009/0158823, published Jun. 25, 2009, and U.S. patent application Ser. No. 12/955,282, filed Nov. 29, 2010, the entire contents of every one of which being incorporated by reference herein as if set forth in full.

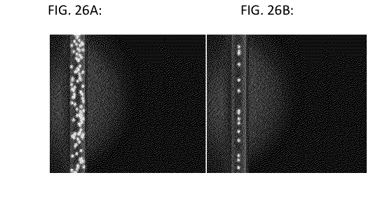

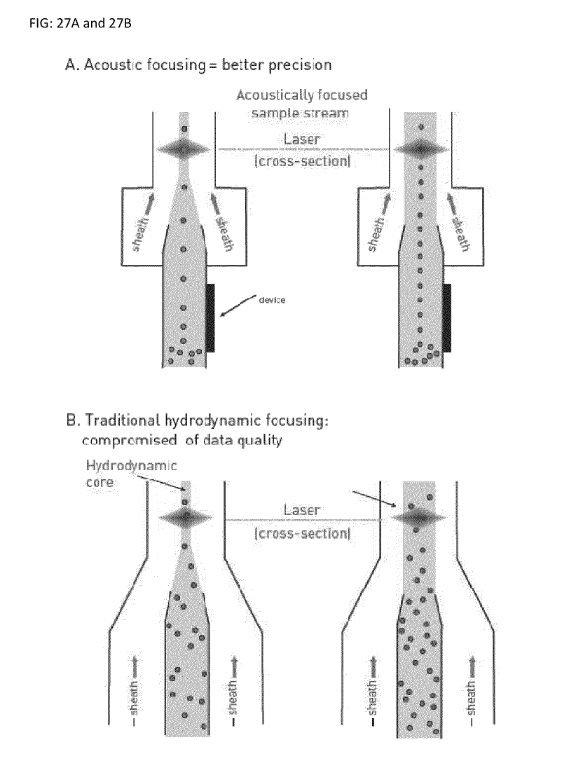

In some embodiments, one exemplary cytometer that may be used in the methods and protocols described herein is the Attune® Acoustic Focusing Cytometer (Life Technologies Corporation) which uses ultrasonic waves (over 2 MHz, similar to those used in medical imaging), rather than hydrodynamic forces, to position cells into a single focused line along the central axis of a capillary (FIGS. 26A and 26B). Acoustic focusing is largely independent of the sample input rate, enabling cells to be tightly focused at the point of laser interrogation regardless of the sample-to-sheath ratio (FIGS. 27A and 27B). This, in turn, allows slowed cell velocity to collect more photons for high-precision analysis at unprecedented volumetric sample throughput. The Attune® cytometer accomplishes all this without high velocity or high volumetric sheath fluid, which can damage cells. In addition, volumetric syringe pumps enable absolute cell counting without beads thereby minimizing cost and sample preparation time. In contrast, cytometers that use hydrodynamic focusing maintain the same sample speed at all flow rates, causing cells to lose focus as the sample core widens to increase flow rate.

FIGS. 26A and 26B depict acoustic focusing in action. Fluorescent microspheres were applied to the capillary system of an acoustic focusing cytometer. Beads flow through randomly without any acoustic focusing (left panel FIG. 26A), i.e., acoustic focusing is off and the sample is unfocused. With the application of acoustic focusing, the beads are focused into a single line (right panel, FIG. 26B), i.e., acoustic focusing is on and the sample is focused.

FIGS. 27A and 27B depicts acoustic focusing vs. traditional hydrodynamic focusing. FIG. 27A: In acoustic focusing, cells remain in tight alignment even at higher sample rates. With this tight alignment, cells pass through the laser beam at its optimal focal point, resulting in less signal variation and improved data quality. FIG. 27B: In traditional hydrodynamic focusing, increasing the sample rate results in widening of the sample core stream. The speed at which cells pass through the laser is not changed, and is determined by the speed of the sheath fluid flow. Cells are distributed throughout the sample core stream because of reduced differential pressure between sample stream and sheath stream, resulting in reduced cell focusing. Cells are not in tight alignment as they pass through the laser beam, resulting in increased signal variation and compromised data quality.

### Various Methods Based on Application of Flow Cytometry:

Various embodiments of the present disclosure describe methods of characterization and analysis of bioparticles comprising using acoustic focusing.

In one embodiment, a method for analyzing bioparticles comprises: acoustically focusing one or more bioparticles through an interrogation zone; optically exciting the one or more bioparticles in the interrogation zone with an excitation source; detecting an optical signal from the bioparticles; and analyzing the optical signal to characterize at least one quality or quantity parameter of the bioparticles.

A bioparticle is a particle or molecule of biological origin and may include without limitation a cell, an organelle, a protein, a peptide, a nucleic acid and/or a virus. Cells that can be analyzed and characterized by the present methods include without limitation prokaryotic cells, eukaryotic cells, bacterial cells, plant cells, fungal cells, phytoplankton cells, picophytoplankton cells, mammalian cells, cancer cells, blood cells, viruses, rare populations of cells (including but not limited to progenitor cells, stem cells, cells that are markers of disease, angiogenisis markers, neovasculatization markers). Proteins that can be analyzed and characterized by the present methods include without limitation peptides, proteins, precursors of proteins, synthetic proteins, proteins with tags attached thereon, enzymes, hormones, growth factors, antibodies, antigens, cell membrane proteins, pathogenic marker proteins, cancer markers. Nucleic acids may comprise without limitation a DNA, genomic DNA, an RNA molecule, triple helical molecules, oligonucleotides and/or polynucleotides.

In some embodiments, a bioparticle to be analyzed is an intrinsically fluorescent bioparticle (such as but not limited to microbial cells, picophytoplankton, algae etc.). In some embodiments, a bioparticle has a component which when excited by an excitation source during acoustic focusing is able to produce an optical signal. In some embodiments, a bioparticle is a labeled bioparticle. Labeled bioparticles or intrinsically fluorescent bioparticles or bioparticles with components that can be excited to produce an optical signal can all produce an optical signal that can be detected when excited during acoustic focusing.

In one embodiment, a method for analyzing labeled bioparticles comprises: acoustically focusing one or more labeled bioparticles through an interrogation zone; optically exciting the one or more labeled bioparticles in the interrogation zone with an excitation source; detecting an optical signal from the labeled bioparticles; and analyzing the optical signal to characterize at least one quality or quantity parameter of the labeled bioparticles.

Analysis of an optical signal provides data regarding one or more of the following: the nature of the bioparticle, the composition of the biomolecule and/or the properties of a biomolecule. The present disclosure describes, in various embodiments, different type of methods of analysis that can be performed on bioparticles and include but are not limited to: cell proliferation analysis, live/dead cell discrimination, cell cycle analysis, basic phenotyping, immunophenotyping, rare-event detection, apoptosis, phagocytosis, pinocytosis, detection of phosphoproteins, detection of one or more cellular markers, detection of one or more intracellular marker, detection of cancer cells, detection of pathological markers on a cell, microbial cell analysis and/or picophytoplankton analysis. Various exemplary descriptions of analysis, detection and/or characterization of various bioparticles using one or more flow cytometers and one or more labels are provided in sections below. However, as will be recognized by the skilled artisan, these examples are merely descriptive examples of embodiments of methods described herein and the present disclosure is not to be construed to be restricted to these exemplary examples. Methods and protocols described herein may be used to assemble kits for analysis of bioparticles and events associated therewith as described herein.

Examples of cells (cellular bioparticles) that may be analyzed and characterized by the present methods include, but are not limited to, Jurkat cells; HL60 promyoblast cells; U266 myeloma cells; mouse splenocytes; mouse blood; HeLa human cervical carcinoma cells; bovine pulmonary artery epithelial (BPAE) cells; 3T3 mouse embryo fibroblast cells; Chinese hamster ovary (CHO) cells; human mesenchymal stem cells (hMSC); E. coli, S. aureus; plasmocytoid dendritic cells; human platelets; human whole blood, red blood cell, nucleated peripheral blood cell, microbial cells; picophytoplankton cells.

The methods based on acoustic focusing described here may have one or more advantages such as: high collection rates; rapid rare event detection, shorter acquisition time; simple methods; and/or a no-lyse preparation protocol and/or a no-wash method both of which eliminate or greatly reduces cell loss of smaller and difficult to obtain samples. Described in sections below are exemplary methods. In some examples discussed below data that is described but not shown expressly as Figures may be found in the provisional U.S. patent applications relied on for priority, i.e., U.S. Provisional Patent Application Ser. No. 61/501,617, entitled “Acoustic Cytometry Methods and Protocols”, filed Jun. 27, 2011, and of U.S. Provisional Patent Application Ser. No. 61/507,975, entitled “Acoustic Cytometry Methods and Protocols”, filed Jul. 14, 2011, the entire contents of which are incorporated herein by reference.

### Cell Proliferation Analysis

In some embodiment methods of the disclosure of analysis of a bioparticle comprises cell proliferation analysis, wherein the bioparticle is a cell or a group of cells. A method of cell proliferation analysis can comprise: subjecting a labeled cell (bioparticle) to a cell proliferation stimulus; acoustically focusing the labeled cell or the group of labeled cells following the cell proliferation stimulus through an interrogation zone; optically exciting the labeled cell with an excitation source in the interrogation zone; detecting an optical signal from the labeled cell; and analyzing the optical signal to characterize at least one quality or quantity parameter of the labeled cells. In some embodiments, this method may be performed on a cell that has intrinsic fluorescent properties and hence the method can be performed on a cell that is not labeled.

In some embodiments, the method can comprise additionally: acoustically focusing the cell prior to subjecting the cell to a cell proliferation stimulus through an interrogation zone; optically exciting the cell with an excitation source in the interrogation zone; detecting an optical signal from the cell prior to subjecting the cell to a cell proliferation stimulus; analyzing the optical signal from the cell prior to subjecting the cell to a cell proliferation stimulus; and comparing the optical signal from the cell prior to subjecting the cell to a cell proliferation stimulus to the optical signal from the cell following subjecting the cell to the cell proliferation stimulus.

Cell proliferation analysis by dye dilution depends on sensitive instrumentation and an extremely bright dye to accurately distinguish fluorescently labeled cells from autofluorescence after several cell divisions. In one example embodiment, the combination of the Attune® Acoustic Focusing Cytometer and Molecular Probes® CellTrace™ Violet dye allows the identification of up to 10 population doublings following cell proliferation stimulation. In this example, CellTrace™ Violet emissions are collected from the violet laser of the Attune® cytometer, and are fully compatible with Green Fluorescent Protein (GFP)—expressing cells for further multiplexing capabilities. The example described here provides superior resolution of population peaks of optical signals that are close together and also provide simplified multiplexing capabilities (such as in one non-limiting example with GFP or other fluorescent protein detection in the cells undergoing cell proliferation). Ten cell divisions were characterized, analyzed and identified with the Attune® Acoustic Focusing Cytometer and the Molecular Probes® CellTrace™ Violet Cell Proliferation Assay. Human peripheral blood mononuclear cells were isolated from whole blood, stained with CellTrace™ Violet (Invitrogen Cat. No. C34557), and stimulated to proliferate in culture. Cells were stained with mouse anti-human CD4 Alexa Fluor® 488 prior to analysis on the Attune® Acoustic Focusing Cytometer at a flow rate of 25 μL/min. A histogram of fluorescence intensity with each peak representing one subsequent generation of proliferating cells was generated (data not shown). To provide statistics about each generation of cells in a population, the fluorescence histogram was further analyzed with proliferation modeling software (ModFit LT™, Verity Software House). Each generation of cells can be represented by a unique peak color and two-dimensional plot allowing the simultaneous analysis of cell proliferation between CD4+ and CD4− cell populations (data not shown).

### Phenotyping and Immunophenotyping

In some embodiment methods of the disclosure of analysis of a bioparticle comprises phenotyping or immunophenotyping and comprises: acoustically focusing one or more labeled bioparticles through an interrogation zone; optically exciting the one or more labeled bioparticles in the interrogation zone with an excitation source; detecting an optical signal from the labeled bioparticles; and analyzing the optical signal to characterize at least one quality or quantity parameter of the labeled bioparticles.

In some embodiments, a method for analyzing labeled bioparticles comprises immunophenotyping analysis which includes labeling bioparticles with one or more conjugated antibodies prior to the acoustic focusing step. Such a method may comprise: labeling bioparticles with one or more conjugated antibodies wherein the bioparticles are cells; acoustically focusing one or more labeled bioparticles through an interrogation zone; optically exciting the one or more labeled bioparticles in the interrogation zone with an excitation source; detecting an optical signal from the labeled bioparticles; and analyzing the optical signal to characterize at least one quality or quantity parameter of the labeled bioparticles. In one embodiment of the method, certain optical signals are indicative of a particular immunophenotype. A method of analyzing an immunophenotype may comprise analysis of cells (bioparticles) such as but not limited to blood cells, human blood cells. In some examples, human blood cells may be immunophenotyped based on the expression of markers such as but not limited to a CD45 marker, a CD3 marker, a CD4 marker, a CD8 marker, a CD19 marker or a CD56 marker. In some examples human blood cells may be immunophenotyped as T-cells, B-cells, NK-cells, CD3 T-cells, CD19B-Cells, CD56-NK cells, CD4 T-helper cells, CD8 T-suppressor cells lymphocytes. In some embodiments, the method may comprise performing a multi-color immunophenotyping.

An example describing a six-color immunophenotyping analysis performed by methods described herein on the Attune® Acoustic Focusing Cytometer is described below for normal human blood cells that were labeled for six-color immunophenotyping with the following directly labeled mouse anti-human antibody conjugates: CD45-Pacific Orange™, CD3-FITC, CD8-Pacific Blue™ CD56-R-PE, CD19-TRI-COLOR® (all from Life Technologies), and CD4-V500 (BD Biosciences) dyes. Gating was performed on CD45-positive lymphocytes to generate bivariate plots. The Attune® Acoustic Focusing Cytometer with red laser option shows excellent segregation of populations in immunophenotyping experiments of up to six colors. There is strong signal separation for more data clarity, and six-color detection is easily performed with the automated compensation module.

Materials: CD3 APC-Alexa Fluor®750 Conjugate (Cat. No. MHCD0327); □CD4 PE-Cy5.5 Conjugate (Cat. No. MHCD0418); CD8 R-PE Conjugate (Cat. No. MHCD0844); □CD19 Alexa Fluor® 647 Conjugate (Cat. No. MHCD1921); CD45 PE-Cy7 Conjugate (Cat. No. MHCD4512); □CD56 Alexa Fluor®488 Conjugate (Cat. No. MHCD5620); Normal Mouse IgG (Cat. No. 10400C); Attune® Acoustic Focusing Cytometer with red laser option; □AbC™ Anti-Mouse Bead Kit (Cat. No. A-10344); □Human peripheral blood collected in anticoagulant; Flow cytometry tubes; Phosphate Buffered Saline (PBS); PBS-BSA (PBS+1% BSA+2 mM Nan3, pH 7.4); □Ammonium chloride lysis buffer.

Methods: Human whole blood was collected with sodium heparin anticoagulant, and ammonium chlorite lysis buffer was used to lyse the red blood cells. Titration of each antibody conjugate was performed for all six conjugates to determine the optimal titer to use for the assay. Fluorescence-Minus-One (FMO) controls were used to determine marker placement. AbC™ anti-mouse beads were used for single-color compensation controls. Normal human blood cells were labeled for six-color immunophenotyping with the following directly labeled mouse anti-human conjugates: CD3 APC-Alexa Fluor®750, CD4 PE-Cy5.5, CD8 PE, CD19 Alexa Fluor®647, CD45 PE-Cy7, and CD56 Alexa Fluor®488. Data acquisition was performed on an Attune® Acoustic Cytometer with red laser option, collecting 10,000 lymphocyte events with the Standard 100 μL/min collection rate. Data analysis was performed using the Attune® Cytometric Software. Gating was performed on CD45-positive lypmphocytes to generate all histogram and bi-variate plots (data not shown).

Results: Complete lymphocyte immunophenotyping is demonstrated with CD3 T-cells, CD19 B-cells, and CD56-Natural Killer (NK) cells. Further T-cell subsets are defined using CD4 for T-helper and CD8 for T-suppressor cells (data not shown). Lin-log scaling is used to display bi-variate plots for improved visualization of the data. The Attune® Acoustic Focusing Cytometer generates expected lymphocyte immunophenotyping results, demonstrating the utility of the instrument.

Another example describing a six-color immunophenotyping analysis performed by methods described herein on the Attune® Acoustic Focusing Cytometer is described below for mouse whole blood cells that were labeled for six-color immunophenotyping

Materials: □Direct conjugated anti-mouse monoclonal antibodies; Rat anti-mouse CD16/32 purified (Cat. No. MFCR00); □Attune® Acoustic Focusing Cytometer with red laser option; AbC™ Anti-Rat/Hamster Bead Kit (Cat. No. A-10389); AbC™ Anti-Mouse Bead Kit (Cat No. A-10344); Mouse peripheral blood collected in anticoagulant; Flow cytometry tubes; PBS (Phosphate Buffered Saline); PBS-BSA (PBS+1% BSA+2 mM NaN3, pH 7.4); Ammonium chloride lysis buffer.

Methods:

Stain Protocol:

1. Block Fc binding receptors by pretreating with 0.1 ug of rat anti-mouse CD16/32 per 10 uL of whole peripheral blood and incubating for a minimum of 10 minutes prior to antibody labeling.
2. Pipet antibody conjugates into labeled sample tubes; the volume should be 30 uL.
3. Add 20 ul of anticoagulated mouse whole blood to the antibody solution and mix well.
4. Incubate protected from light for 30 minutes (or reagent manufacturer's recommendation).
5. Add 3 ml ammonium chloride lysis buffer to the tubes and incubate for 10 minutes at room temperature.
6. Centrifuge at 400×g for 5 minutes
7. Carefully discard the supernatant with disturbing the pelleted cells.
8. Wash 1× with PBS.
9. Resuspend the cells in 1 ml of PBS

Compensation Controls:

1. Add one drop of AbC™ anti-Rat/Hamster (or anti-Mouse depending on host species of monoclonal) to a labeled sample tube for each antibody conjugate included in the panel.
2. Add the recommended amount of each antibody conjugate to the AbC™ Capture Beads.
3. Incubate for 15 minutes at room temperature, protected from light.
4. Add 3 ml PBS and centrifuge for 5 minutes at 200 g.
5. Carefully remove the supernatant and resuspend the bead pellet by adding 1 ml PBS.
6. Prepare one AbC™ Contro Beads (Component B) sample tube along with 1 ml PBS.

Data Collection:

1. Create a new experiment using instrument settings optimized for mouse lysed whole blood.
2. Create compensation controls for each fluorescence parameter.
3. Modify the workspace to include a FSC vs SSC bivariant plot with a polygon gate to select on the lymphocyte population.

4. Create additional bivariant plots to analyze the subpopulations as shown in FIGS. 1 and 2.

5. Optimize PMT voltages using an unstained sample of the blood. Use of unstained compensation control sample is convenient for this purpose as it provides histograms for each parameter. Adjust the P1 gate to include lymphocytes and adjust PMT voltages to place cell populations at the desired autofluorescence levels (nominally around 100-1000).

6. Run the compensation controls after optimizing the PMT voltages.

7. Select a first specimen and sample

8. Set the recording conditions to collect 20,000 lymphocyte events at an collection rate of 100-200 μL/min.

9. Proceed with collecting data for samples.

Results: The major lymphocyte T cell subpopulations, B cells, and NK1.1 expressing cells are first identified using bivariant plots (data not shown). The NK1.1 expressing cells are further classified into NK and NKT cells by generating NK1.1 vs CD11c child bivariant plots on non T,B cell population (CD3-19−) and the T cell populations CD4+, CD8+ plus DN (double negative). The Attune® Acoustic Focusing Cytometer shows excellent segregation of populations in immunophenotyping experiments (with up to 6 colors) and has the following advantages: Six-color detection with minimal compensation; Strong signal separation for more data clarity; and Less need for difficult tandem dyes.

### Detection of Phosphoproteins

Some embodiments describe methods for detecting phosphoproteins on a cell disposed within a fluid medium, comprising: stimulating or inhibiting the cell with a kinase or a kinase inhibitor respectively to phorsporylate or de-phosphorylate one or more proteins on the cell; contacting the cell with one or more antibody specific to detect the one or more phosphorylated protein; acoustically focusing the cell in the fluid medium; optically exciting the cell with an excitation source; detecting an optical signal from the cell; and analyzing the optical signal, wherein the optical signal is indicative of the presence or absence of the one or more phosphorylated protein.

One illustrative example for detecting phosphoproteins on a cell is detection of mitogen-activated protein kinase (MAPK) which is important for investigations of human diseases such as cancer. MAPK signaling cascades play important roles in the critical decision processes within a cell, including cellular responses to environmental stimuli and disease progression. MAPKs regulate diverse cellular programs including embryogenesis, proliferation, differentiation, and apoptosis based on cues derived from the cell surface and on the metabolic and environmental state of the cell. Multiparameter flow cytometry provides an important tool for dissecting signaling pathways in cell populations using intracellular staining with fluorescent antibodies against phosphorylation site-specific proteins. While reagents and techniques aimed at phosphoprotein-specific detection have progressed, the signals that result from these experiments are usually dim and difficult to distinguish. Advancements in instrumentation using acoustic focusing allow improved detection of dim signals. The Attune® Acoustic Focusing Cytometer employs high-frequency sound waves to maintain a tightly focused sample stream, allowing greater precision at the laser interrogation point. By using the High Sensitive transit time setting to slow the sample stream, longer laser interrogation time is permitted, which increases the sensitivity of detection.

In a specific example here three phosphoproteins, Akt, Erk1/2, and P38, were analyzed using flow cytometry methods as described here using the Attune® Acoustic Focusing Cytometer, which enables the detection of dim signals through highly sensitive and precise data gathering capabilities. Typically signals that result from these experiments are usually dim and difficult to distinguish. Advancements in instrumentation using acoustic focusing allow improved detection of dim signals. The Attune® Acoustic Focusing Cytometer employs high-frequency sound waves to maintain a tightly focused sample stream, allowing greater precision at the laser interrogation point. By using the High Sensitive transit time setting to slow the sample stream, longer laser interrogation time is permitted, which increases the sensitivity of detection.

Jurkat cells were treated with LY294002 and stained with Akt Alexa Fluor® 488 direct conjugate, comparing all collection rates on the Attune® Acoustic Focusing Cytometer to the low flow rate of the LSRII™ and FACSCalibur™ instruments. Red traces represent the unstained, untreated Jurkat cells, purple traces represent untreated, Akt Alexa Fluor® 488 stained Jurkat cells, blue traces represent LY294002 treated Akt Alexa Fluor® 488 stained Jurkat cells (data not shown). Improved separation, demonstrated by higher SI values, of LY294002 treated vs. untreated cells stained with Akt Alexa Fluor® 488 is observed at higher standard collection rates on the Attune® Acoustic Focusing Cytometer as compared to conventional cytometers using hydrodynamic focusing. This allows for faster collection while maintaining data integrity.

Jurkat cells were also treated with PMA/ionomycin and stained with Erk1/2 Alexa Fluor® 488 direct conjugate, comparing the High Sensitive and Standard transit times (each using 25 μL/min sample injection rate) on the Attune® Acoustic Focusing Cytometer to the low flow rate (12 μL/min) of the LSRII™ and FACSCalibur™ instruments. Purple traces represent untreated, Erk1/2 Alexa Fluor® 488 stained Jurkat cells, blue traces represent PMA/ionomycin treated Erk1/2 Alexa Fluor® 488 stained Jurkat cells (data not shown). The Attune® Acoustic Focusing Cytometer demonstrates improved separation of low-expressed proteins using the Highly Sensitive mode as compared to the conventional instruments using hydrodynamic focusing.

Jurkat cells treated with anisomycin and stained with P38 Alexa Fluor® 488 direct conjugate, comparing the High Sensitive and Standard transit times (each using 25 μL/min) of the Attune® Acoustic Focusing Cytometer to the low flow rate (12 μL/min) of the LSRII™ and FACSCalibur™ instruments. Purple traces represent untreated, P38 Alexa Fluor® 488 stained Jurkat cells, blue traces represent anisomycin treated P38 Alexa Fluor® 488 stained Jurkat cells (data not shown). The Attune® Acoustic Focusing Cytometer demonstrates improved separation of low expressed proteins using the High-Sensitive mode as compared to the conventional instruments using hydrodynamic focusing.

### Detection of Fluorescent Protein Expression

In some embodiments, a method for analyzing labeled bioparticles comprises detecting fluorescent protein detection in a cell, wherein the bioparticle is a cell. A method for detecting fluorescent protein expression on a cell disposed within a fluid medium, comprises: transfecting the cell with one or more fluorescent proteins; acoustically focusing the cell in the fluid medium; optically exciting the cell with an excitation source; detecting one or more optical signals from the cell; and analyzing the optical signal, wherein the detection of an optical signal corresponding to one or more fluorescent protein is indicative of the presence of expression of the one or more fluorescent proteins and the absence of an optical signal corresponding to one or more fluorescent protein is indicative of the absence of expression of the fluorescent protein.

In some embodiments of this method, the detection of an optical signal corresponding to one or more fluorescent protein is indicative of successful transfection of the fluorescent protein (and any fusion protein/peptide attached thereto). In some embodiments, detection of a first optical signal corresponding to a first fluorescent protein and the detection of a second optical signal corresponding to a second fluorescent protein is indicative of transfection of the cell by the first and the second fluorescent proteins. In some embodiments, analyzing the optical signal further comprises analyzing the percentage of cells transfected with the one or more fluorescent proteins. Some embodiments therefore relate to quantifying the number of cells transfected. Various fluorescent proteins may be detected and analyzed by the methods described herein and can be but are not limited to a red fluorescent protein, a green fluorescent protein, a blue fluorescent protein, a yellow fluorescent protein.

### GFP Detection:

An example for the detection of green fluorescent protein using flow cytometry is described here using BacMam CellLight® reagents which are fluorescent protein-signal peptide fusions that provide accurate and specific targeting to cellular structures for live-cell applications, here used with human osteosarcoma (U2OS) cells. In this study, the ability to detect Green Fluorescent Protein (GFP) with the Attune® Acoustic Focusing Cytometer is demonstrated, and can be used to provide correlative imaging results, and also to demonstrate the ability of the Attune® Cytometric Software to easily create overlay plots.

Method: U2OS cells (1×105) were plated in separate 10 cm tissue culture dishes in complete medium. Including an unlabeled control, various GFP-expressing BacMam CellLight® reagents were added at 2% v/v and incubated for 24 hr (FIGS. 1 and 2). In parallel, cells were plated into 6 separate dishes and labeled with histone 2B (H2B)-GFP at concentrations of 10%, 5%, 2%, 1%, 0.5%, and 0.2% v/v, and incubated for 24 hr. Data was generated from cells that were transduced with H2B-RFP and mitochondria-GFP simultaneously (2% v/v each). After 24 hr, all cells were imaged, harvested, and resuspended in PBS at a concentration of 1×106 cells/mL for analysis on the Attune® Acoustic Focusing Cytometer.

Samples were acquired and analyzed on the Attune® cytometer using a 488 nm laser with 530/30 bandpass filters for GFP and 575/24 bandpass filters for RFP. The main population of cells was gated to exclude debris, and 50,000 gated events were collected at a rate of 200 μL/min in standard sensitivity mode. For single GFP results, histograms were generated and sample analysis with overlay plots was performed using the Attune® Cytometric Software. A dual-parameter plot was generated for the dual-expressing GFP and RFP cells.

U2OS cells labeled with H2B-GFP BacMam CellLight® reagent at various concentrations and measured with the Attune® Acoustic Focusing Cytometer. The overlay plot, made with the Attune® Cytometric Software, displays unlabeled cells and the transduced GFP-expressing cells. U2OS cells transduced with a dilution series of H2B-GFP BacMam CellLight® reagents showed corresponding levels of GFP expression, from brightest to dimmest as follows: 10% v/v (navy), 5% v/v (gray), 2% v/v (aqua), 1% v/v (pink), 0.5% v/v (green), 0.2% v/v (red), and untransduced (black). An inverse relationship exists where the unstained population for each transduction increases as the amount of CellLight® reagent introduced decreases. Fluorescence quantitation data were obtained from analysis of the various cell populations using the Attune® Acoustic Focusing Cytometer.

Visual confirmation of GFP expression from dilution series transduction. Prior to analysis on the Attune® Acoustic Focusing Cytometer, samples of each of the transduced cell populations were visualized using fluorescence microscopy to confirm GFP expression. Images were generated using a Zeiss microscope with an Alexa Fluor® 488 dye filter (10× magnification and 100 ms). (A) 10%, (B) 5%, (C) 2%, (D) 1%, (E) 0.5%, and (F) 0.2%.

Sensitive detection of GFP expression: The Attune® Acoustic Focusing Cytometer delivers rapid and sensitive analysis of GFP-expressing and GFP/RFP co-expressing cell populations, providing a quick and reliable method to quantitatively evaluate cell transfection with fluorescent proteins. Overlay plots can be made directly in the Attune® Cytometric Software by using the simple drag-and-drop feature, where the legend displayed above the overlay plot has the names of each sample in the corresponding color.

U2OS cells labeled with various GFP-expressing BacMam CellLight® reagents and measured with the Attune® cytometer. Overlay plots, made with the Attune® Cytometric Software, display unlabeled cells and the transduced GFP-expressing cells. (A) Cells labeled with histone-2B GFP are divided into a large population of cells expressing GFP and a smaller segment that exhibits fluorescence slightly brighter than the unlabeled control. (B) Cells labeled with mitochondria-GFP separate into a large population of cells brightly expressing GFP and a very small population dimly expressing GFP (positioned to the right of the main peak next to the unlabeled sample). (C) Cells labeled with cytoskeleton-GFP show almost equal numbers of bright and dim GFP-expressing cells. (D) Cells labeled with golgi-GFP reveal a majority of the population to be expressing GFP and a very small population of dimly expressing cells. (E) Cells labeled with peroxisome-GFP exhibit a large, brightly fluorescent population and a minor population of dim GFP-expressing cells (data not shown). Visual confirmation of GFP expression in cells, Prior to analysis on the Attune® Acoustic Focusing Cytometer, samples of each of the transduced cell populations were visualized using fluorescence microscopy to confirm GFP expression. (A) Histone-2B GFP; (B) Mito-GFP; (C) Cyto-GFP; (D) Gold GFP; and (E) Peroxi-GFP. Images were generated on a Zeiss Horoscope using 10× magnification and 100 ms (data not shown),

RFP Detection: Another example for the detection of red fluorescent protein (RFP) using flow cytometry is described here using similar methodology as described above for GFP is described here.

Method: U2OS cells (1×105) were plated in separate 10 cm tissue culture dishes in complete medium. Including an unlabeled control, various RFP-expressing BacMam CellLight® reagents were added at 2% v/v and incubated for 24 hr. In parallel, cells were plated into 6 separate dishes and labeled with histone 2B (H2B)-RFP at concentrations of 10%, 5%, 2%, 1%, 0.5%, and 0.2% v/v, and incubated for 24 hr. Data was generated from cells that were transduced with H2B-RFP and mitochondria-GFP simultaneously (2% v/v each). After 24 hours, all cells were imaged, harvested, and resuspended in PBS at a concentration of 1×106 cells/mL for analysis on the Attune® cytometer. Samples were acquired and analyzed on the Attune® Acoustic Focusing Cytometer using a 488 nm laser with bandpass filters 530/30 for GFP and 575/24 for RFP. The main population of cells was gated to exclude debris, and 50,000 gated events were collected at a rate of 200 μL/min in standard sensitivity mode. For single RFP results, histograms were generated and sample analysis with overlay plots was performed using the Attune® Cytometric Software. A dual-parameter plot was generated for the dual GFP- and RFP-expressing cells (data not shown).

U2OS cells labeled with various RFP-expressing BacMam CellLight® reagents and measured by the Attune® cytometer. Overlay plots for unlabeled cells and the transduced RFP-expressing cells were made directly in the Attune® Cytometric Software. (A) Cells labeled with Golgi-RFP appear with a relatively tight fluorescence profile, with the tail of the main peak overlapping the unlabeled control. (B) Cells labeled with Mito-RFP exhibit a broader population of RFP-expressing cells, with a small population of dimly expressing RFP cells just inside the right shoulder of the unlabeled sample. (C) Cells labeled with Tubulin-RFP form a symmetrical distribution of fluorescent cells (with the dim shoulder coincident with the unlabeled control). (D) Cells labeled with Endo-RFP exhibit the widest distribution of RFP expression of the four constructs tested.

Visual confirmation of RFP-expressing cells. Prior to analysis on the Attune® Acoustic Focusing Cytometer, samples of each of the transduced cell populations were visualized using fluorescence microscopy to confirm RFP expression. (A) Golgi-2B RFP; (B) Mito-RFP; (C) Tubulin-RFP; and (D) Endo-RFP. Images were generated on a Zeiss microscope using 10× magnification and 100 ms.

U2OS cells labeled with H2B-RFP BacMam CellLight® reagent at various concentrations and measured with the Attune® Acoustic Focusing Cytometer. An overlay plot for unlabeled cells and the transduced RFP-expressing cells was made directly in the Attune® Cytometric Software. Cells were transduced using a dilution series of H2B-RFP BacMam CellLight® reagent as follows: 10% v/v (navy), 5% v/v (gray), 2% v/v (aqua), 1% v/v (pink), 0.5% v/v (green), 0.2% v/v (red), and untransduced (black). The cells showed corresponding levels of RFP expression. An inverse relationship exists where the unstained population for each transduction increases as the amount of BacMam introduced decreases. Fluorescence quantitation data were obtained for each cell population using the Attune® Acoustic Focusing Cytometer.

Visual confirmation of RFP-expressing cells from dilution series transductions. Prior to analysis on the Attune® Acoustic Focusing Cytometer, samples of each of the transduced cell populations were visualized using fluorescence microscopy to confirm RFP expression. Images were generated using a Zeiss microscope with an RFP filter (10× magnification and 100 ms). The percentages shown on the panels correspond to the concentrations of CellLight® reagent used for transduction,

Sensitive detection of RFP expression: The Attune® Acoustic Focusing Cytometer delivers rapid and sensitive analysis of GFP-expressing and GFP/RFP-co-expressing cell populations, providing a quick and reliable method to quantitatively evaluate cell transfection with fluorescent proteins. Overlay plots can be made directly in the Attune® Cytometric Software by using the simple drag-and-drop feature, where the legend displayed above the overlay plot has the names of each sample in the corresponding color.

Detection of both GFP and RFP co-expression: In some embodiments a method may comprise simultaneous detection of multiple fluorescent proteins that a cell is co-labeled and/or co-transfected with. For example, U2OS cells labeled with GFP- and RFP-expressing BacMam CellLight® reagents and measured with the Attune® Acoustic Focusing Cytometer. Cells were simultaneously labeled with Mito-GFP and H2B-RFP at 2% v/v each. (A) A dual-parameter plot shows that a large population is co-labeled with both RFP and GFP; however, a significant percentage is also unlabeled and/or expressing more GFP than RFP. (B) The same sample was imaged prior to flow analysis and confirms co-expression of both fluorescent proteins. Image was generated using a Zeiss microscope with 50 ms GFP and 250 ms RFP, 10× magnification (image not shown).

Detection and Discrimination of Natural Populations of Prochlorococcus spp. and Synechococcus spp. in Environmental Samples: Flow cytometry methods described here are useful for studying the biology, ecology, and biogeochemistry of marine photosynthetic picoplankton. Populations of photosynthetic picoplankton are intrinsically fluorescent due to their photopigment content, and differences in photopigment composition are used to distinguish the various groups. Prochlorococcus spp. and Synechococcus spp. are the two major groups of microbes that comprise photosynthetic picoplankton and have been extensively studied for their principal role in primary production. Prochlorococcus spp. are the smallest and most abundant photosynthetic organisms known, and, along with Synechococcus spp., have a large impact on the global carbon cycle. Prochlorococcus spp. are approximately 0.6 Mm in size and contain the red-fluorescent molecules divinyl-chlorophylls a and b. At 1 Mm, cells of Synechococcus spp. are larger and contain the orange-fluorescent phycoerythrin in addition to red-fluorescent chlorophyll. These differences allow the present methods to detect and discriminate between natural populations of Prochlorococcus spp. and Synechococcus spp. in environmental samples.

Conventional cytometers employ large sheath-to-sample flow rates to hydrodynamically focus particles. In contrast, the Attune® Acoustic Focusing Cytometer uses ultrasonic waves to focus particles and requires significantly lower sheath fluid flow rates. The Sensitive mode on the Attune® cytometer further reduces the instrument sheath flow rate, thereby slowing the particle velocity. By slowing the particle velocity, a researcher can increase the laser interrogation and photon collection times for dim, low-background populations (e.g., the inherently dimly fluorescent Prochlorococcus spp. from oligotrophic surface water samples). The 405 nm laser enables better excitation of divinylchlorophylls from Prochlorococcus spp. and enhances separation of distinct picophytoplankton populations from background signal. Syringe driven sample fluidics permits the direct counting of cells in a given population. Combining syringe-driven sample handling with excitation of divinyl-chlorophylls with the 405 nm laser allows for direct enumeration of Prochlorococcus spp. in SYBR® Green I-stained samples.

### Detection of a Rare Event

In some embodiments, a method of the disclosure for analysis of a labeled bioparticle comprises detection of a rare event in a population of cells, wherein the labeled bioparticle is the population of cells. In one embodiment, a method for detection a rare event within a population of cells, the method comprises: acoustically focusing the population of cells; optically exciting the population of cells with an excitation source; detecting one or more optical signals from the population of cells; and analyzing the optical signal, wherein the detection of an optical signal corresponding to a rare event is indicative of the presence of the rare event and the absence of an optical signal corresponding to a rare event is indicative of the absence of the rare event.

In some embodiments, the rare event is the detection of a rare subset of cells within a population of cells. In some embodiments, a rare subset of cells comprises less than 5% the population of cells. In some embodiments, a rare subset of cells may comprise less than 0.5% of the total cell population.

In some embodiments a rare subset of cells comprises from about less than 0.5% to about 5% of a total cell population, and may include 0.4%, 0.5%, 0.6%, 0.7%, 0.8%, 0.9%, 1%, 1.5%, 2%, 2.5%, 3%, 3.5%, 4%, 4.5% and 5% of the total cells. In some embodiments, detection of a rare subset of cells can comprise detecting from about 1 cell to about 20 cells per milliliter of cell sample. For example, a rare subset may be 1 cell, 2 cells, 3 cells, 4 cells, 5 cells, 6 cells, 7 cells, 8 cells, 9 cells, 10 cells, 11 cells, 12 cells, 13, 14 cells, 15 cells, 16 cells, 17 cells, 18 cells, 19 cells, or 20 cells in one milliliter of a sample, such as a blood sample, a lymph sample, a bone marrow sample, a plasma sample, a bodily fluid sample, or a cell sample.

A method may additionally further comprise identification of the rare subset of cells (such as for example by phenotyping, immunophenotyping and/or biomarker identification). Examples of rare cell subsets that may be detected by the present methods include detection of plasmocytoid dendritic cells (pDc). Plasmocytoid dendritic cells are rare cells that produce type I interferon in response to viruses and comprise less than 0.5% of the total splenocyte population. Other examples of subsets of rare cells include, but are not limited to, human mesenchymal cells, CD34+ cells in a population of peripheral blood cells; angiogenic cells in human blood, circulating endothelial cells in human blood; and/or circulating hematopoietic progenitor cells in human blood. Some illustrative examples are described below describing examples of rare cell subsets that may be detected, analyzed and/or identified by the present methods. One of skill in the art, in light of the present teachings, will realize that the present methods are not limited to the illustrated example rare cells but may be used for the identification of any rare subset of cells.

Detection of Plasmocytoid dendritic Cells and Circulating Endothelial Cells: Analysis of rare cell populations by flow cytometry requires the collection of high numbers of events in order to attain a reliable measure of accuracy, which leads to long acquisition times using traditional hydrodynamic focusing. An accurate measurement of a population with a 1/1,000 probability requires the collection of 1.6×10⁶ events to detect the population with a CV of 2.5%. When the probability of event decreases to 1/10,000 the number of events needed for a CV of 2.5% increases to 1.6×10. The ability of acoustic cytometry to run samples at up to 1000 mL/min while maintaining precision and accuracy provides a distinct advantage over typical hydrodynamic focusing cytometers for collecting such large samples. Two panels were presently tested which demonstrate the ability of the present methods using acoustic cytometry to make sensitive and accurate measurements while greatly increasing the speed to acquire very large sample volumes.

pDCs Detection: First, a panel was developed to detect mouse plasmacytoid dendritic cells (pDCs). This specialized cell population that produces large amounts of type I interferons in response to viruses, and typically comprise less than 0.5% of the total splenocyte population in naïve mice. pDCs can be further identified by additional methods described herein such as methods for immunophenotyping based on their immunophenotype CD19−, B220high, CD317+.4

Method: A single cell suspension of mouse splenocytes were blocked with CD16/32. Cells were then stained with CD19-Pacific Blue™, CD317-Alexa Fluor® 488, CD45R/B220-PE direct conjugates and SYTOX® AADvanced™ Dead Cell Stain. A gate was made on live cells using SYTOX® AADvanced™ Dead Cell Stain, followed by gating on CD19 negative cells. A two parameter plot of CD45R/B220 vs. CD317 was used to identify pDCs. A collection rate of 500 μl/min was used to acquire 1.3 million total cells. Plasmacytoid dendritic cells were identified as dual B220+/CD317+ (upper right quadrant), and compromise 0.851% of live CD19− cells, which is 0.194% of total splenocytes. Total acquisition time was 23 minutes, three times faster than the same sample run on a traditional hydrodynamic focusing cytometer.

CEC Detection: A second panel was developed to detect extremely rare circulating endothelial cells (CECs) which have a typical range for healthy individuals of 1×10⁻⁷ to 1×10⁻⁵ CEC per leukocyte (1-20 cells/mL of venous blood). To reduce the risk of loss of CECs in processing, a no-lyse, no-wash procedure was developed which requires the speed and accuracy of acoustic focusing to process such dilute samples.

Identification of CD34+ cells from peripheral blood: Peripheral blood from a normal donor was stained and run on the Attune® Acoustic Focusing Cytometer at a collection rate of 1,000 μL/min with a stop gate set at 500,000 total cells. A rare population of 0.045% CD34+ cells was identified within the population of cells with an acquisition time of 4 minutes, 28 seconds (data not shown).

### Cell Cycle Analysis

In some embodiments, a method for analyzing labeled bioparticles comprises analyzing different phases of cell cycle of a cellular bioparticle and comprises: acoustically focusing one or more labeled cells for which cell cycle analysis is sought through an interrogation zone; optically exciting the one or more labeled cells in the interrogation zone with an excitation source; detecting an optical signal from the labeled cells; and analyzing the optical signal to characterize at least one quality or quantity parameter of the labeled cells, wherein different optical signals correspond to different cell cycle phases. In some embodiments, a method may additionally comprise quantitating the percentage of cells in one or more cell cycle phases.

Cell cycle analysis is another example of an embodiment method of the disclosure based on precisely detecting differences in fluorescence intensity between multiple cell populations. With the Attune® Acoustic Focusing Cytometer, minimal variation in results is seen regardless of sample throughput rate. Percent CV of G0/G1 at different flow rates on the Attune® cytometer and a competitor (C) at different sample rates. Note the minimal change in variability (% CV) for the Attune® Acoustic Focusing Cytometer, even at a high sample rate. In one example, Jurkat cells were fixed and stained with propidium iodide, treated with RNase, and analyzed at a concentration of 1×106 cells/mL on a high-end instrument that uses hydrodynamic focusing, and also on the Attune® Acoustic Focusing Cytometer at different sample rates. Cells in G0/G1 phase and cells in G2/M phase were detected using both instruments. As sample rates increased on the instrument that uses hydrodynamic focusing, the width of the G0/G1 and G2/M peaks increase, whereas for the Attune® cytometer the peaks are relatively stable, even at the highest sample rate of 1,000 μL/min (data not shown).

Human Mesenchymal Cell: Adult human mesenchymal stem cells (hMSCs) are rare fibroblast-like cells capable of differentiating into a variety of cell tissues, including bone, cartilage, muscle, ligament, tendon, and adipose. The International Society for Cellular Therapy (ISCT) has proposed a set of standards to define hMSCs for laboratory investigations and preclinical studies: adherence to plastic in standard culture conditions; in vitro differentiation into osteoblasts, adipocytes, and chondroblasts; and specific surface antigen expression in which P95% of the cells express the antigens recognized by CD105, CD73, and CD90, with the same cells lacking (Y2% positive) the antigens recognized by CD45, CD34, CD14 or CD11b, CD79a or CD19, and HLA-DR. Recent studies have shown that CD34 antigen may be present, but its expression is transient and present only in early passages of cells derived from some isolates. Direct measurement of proliferation combined with simultaneous detection of the ISCT-consensus immunophenotypic profile provides data that are used to determine the differentiation status and health of the cells. Here two examples of immunophenotyping and cell cycle analysis are shown.

Materials and Methods: hMSCs (derived from normal human bone marrow) at passage 7 were fixed with 2% formaldehyde, blocked using normal mouse serum, stained in three tubes for 30 minutes at room temperature in the dark, and washed and suspended in PBS. Compensation was set using the AbC™ Anti-Mouse Bead Kit (Invitrogen Cat. No. A10344). Fluorescence-minus-one (FMO) controls were used for gate placement. Samples were then acquired on the Attune® cytometer using a 405 nm laser with a 450/40 bandpass and 603/48 bandpass, and a 488 nm laser with a 530/30 bandpass, 575/24 bandpass, and 640 longpass. The panel configurations are as follows:
CD73 PE, CD19 Pacific Blue™, CD45 Pacific Orange™ direct conjugates
CD90 FITC, CD34 PE, CD14 Pacific Blue™ direct conjugates
CD105 PerCP-Cy®5.5, HLA-DR Pacific Blue™ direct conjugates

Undifferentiated hMSCs, passage 7 (hMSC P7), demonstrated a decrease in percentage in S phase as the cells remain in culture and become more confluent over time. Subculturing prior to 80% confluency or 3-5 days is suggested for an optimized growth rate without differentiation. After 72 hr, the percentage in S phase is reduced significantly, revealing a decrease in DNA replication and indicating cells should be subcultured according to recommended criteria. At 0 hr, hMSC P7 were plated into separate tissue culture dishes with DMEM, 10% hMSC FBS, and 2 mM L-glutamine. Cells were harvested using TrypLE™ Express at 24, 72, 96, and 120 hr, fixed in 70% EtOH, and stored at −20° C. For analysis, cells were washed and resuspended in DPBS with 0.1% Triton® X-100. Cells were adjusted to a concentration of 105/mL using the Countess® Automated Cell Counter and labeled with 500 nM FxCycle™ Violet stain. Samples were analyzed on the Attune® Acoustic Focusing Cytometer, and ModFit LT™ (Verity Software House) curve-fitting software was used to extract the cell cycle phase distributions. All phases of the cell cycle detected in hMSCs using FxCycle™ Violet stain and the Attune® Acoustic Focusing Cytometer. Cells were analyzed at 96 hr without subculturing. A significant decrease in percentage of cells in S phase as culture time is extended indicates a reduction in growth rate and emphasizes the need for earlier subculturing to optimize growth rate and nondifferentiation.

### Microbial Cell Analysis

In some embodiments, a method for analyzing microbial bioparticles comprises analyzing different microbial cellular events of a microbial bioparticle and comprises: acoustically focusing one or more microbial cells through an interrogation zone; optically exciting the one or more microbial cells in the interrogation zone with an excitation source; detecting an optical signal from the microbial cells; and analyzing the optical signal to characterize at least one quality or quantity parameter of the microbial cells, wherein different optical signals correspond to different types of microbial events.

Several microbes have intrinsic fluorescence also referred to as natural fluorescence and this property has been used in some embodiments of microbial analysis methods described in the present disclosure using acoustic focusing flow cytometry methods. In embodiments, where microbes may not be naturally fluorescent the microbe can be labeled prior to the acoustic focusing.

In some embodiments, detection of one or more optical signals are indicative of microbial cell events such as but not limited to microbial viability, number of microbial cells, detection of gram positive status of a microbe, detection of gram negative status of a microbe, microbial membrane potential, microbial metabolism and combinations thereof. In some embodiments, detecting and/or analyzing microbial viability comprises detecting live microbial cells separately from dead microbial cells.

Some example embodiments are described below using flow cytometry methods described herein such as, detection and quantification of viable and non-culturable organisms, analysis of host-microbe interactions, analysis of microbial cell cycle, and detailed spatial and temporal analysis of microbial metabolism in different environments. In some examples, methods of the present disclosure performed on the Attune® Acoustic Focusing Cytometer (Life Technologies) allow complete cytometric analysis of microbial physiology. The Attune® Acoustic Focusing Cytometer offers many advantages over traditional hydrodynamic focusing cytometers, including precise alignment of particles at increased collection rates (up to 1,000 TL/minute). Consistent fluorescence emission were detected in samples of fluorescently labeled Staphylococcus aureus (S. aureus) analyzed at all collection rates using the Attune® cytometer. In addition, the Attune® cytometer is a valuable tool for cell vitality assessment, membrane potential measurement, and cell viability assays. Consistent fluorescent detection at flow rates from 25 8 L/min to 1,000 8 L/min. S. aureus cells were stained with SYTO® 9 (Cat. No. S34854) and analyzed on the Attune® Acoustic Focusing Cytometer using 488 nm excitation and the 530/30 bandpass filter (BL1) to collect SYTO® 9 fluorescence emission. (A) Typical scatter observed using a BL1 fluorescence threshold. S. aureus cells can be shown in a color (green) and have a greater forward scatter signal than electronic noise/debris. (B) Fluorescence histogram overlay indicating SYTO® 9 fluorescence of the S. aureus population identified in (A), collected at Sensitive 25 TL/min (red), Sensitive 100 TL/min (blue), Standard 25 TL/min (green), Standard 100 TL/min (black), Standard 200 TL/min (purple), Standard 500 TL/min (burgundy), and Standard 1,000 TL/min (orange) collection rates. Unstained cells are can be shown in another color (grey), collected at Standard 25 TL/min. Little variation was observed across all collection rates (data not shown).

Analysis of relative cell viability within a bacterial culture using flow cytometry: In one example embodiment, Escherichia coli (E. coli) cells were stained with the LIVE/DEAD® BacLight™ Viability Kit (Cat. No. L7012) before analysis using the Attune® Acoustic Focusing Cytometer equipped with 488 nm laser for SYTO® 9 and propidium iodide excitation. Samples were run at a collection rate of Standard 25 TL/min, and fluorescence emission was detected using a 530/30 bandpass filter for SYTO® 9 fluorescence and 640 longpass filter for propidium iodide fluorescence. Both live (L) and dead (D) cells fluoresce green (SYTO® 9) but only dead cells fluoresce red.

Analysis of relative cell vitality within a bacterial culture using flow cytometry: In one example embodiment, untreated E. coli cells and cells treated with an electron transport chain uncoupler (sodium azide) were stained with the BacLight™ RedoxSensor™ Green Vitality Kit (Cat. No. B34954) before analysis using the Attune® Acoustic Focusing Cytometer equipped with 488 nm laser. Samples were run at a collection rate of Standard 25 TL/min, and fluorescence emission was detected using a 530/30 bandpass filter for BacLight™ RedoxSensor™ Green fluorescence. The histogram overlay indicates untreated cells have a brighter green fluorescence and greater redox potential than those treated with sodium azide (data not shown). Analysis of relative membrane potential in an S. aureus culture before and after disruption with a proton ionophore. S. aureus cells were diluted to ˜1×106 CFU/mL in PBS prior to staining with the BacLight™ Bacterial Membrane Potential Kit (Cat. No. B34950) and 20 TM SYTOX® Blue (Cat. No. S34862). Samples stained with 30 TM 3,3′-diethyloxacarbocyanine iodide (DiOC2) alone, and samples stained with DiOC2 and treated with 5 TM carbonylcyanide 3-chlorophenylhydrazone (CCCP, for disruption of membrane potential), were analyzed on the Attune® Acoustic Focusing Cytometer equipped with 488 nm laser for DiOC2 fluorescence excitation. At increased membrane potential, DiOC2 molecules self-associate in the cytosol and shift DiOC2 fluorescence emission from green (detected in the BL1 channel using a 530/30 bandpass filter) to red (detected in the BL3 channel using a 640 longpass filter). In this example, dead cells have been removed from analysis by excluding SYTOX® Blue-positive cells from analysis. A dot plot overlay indicates increased red-shifted DiOC2 fluorescence in the untreated sample (−CCCP, green) as compared to the CCCP-treated sample (+CCCP, red) (data not shown).

Staining of bacteria using BacLight™ Green: In one example embodiment, untreated and alcohol fixed E. coli (A) and S. aureus (B) cells were stained with BacLight™ Green (Cat. No. B35000) before analysis using the Attune® Acoustic Focusing Cytometer equipped with 488 nm laser. Samples were run at a collection rate of Standard 25 TL/min, and fluorescence emission was detected using a 530/30 bandpass filter for BacLight™ Green fluorescence. The histogram overlays indicate that both untreated (L) and alcohol-fixed (F) gram-negative (E. coli) or gram-positive (S. aureus) cells have increased fluorescence over unstained (U) cells when stained with BacLight™ Green. Fluorescence staining of fixed cells is greater than staining in both unfixed and unstained cells.

### Detection of Apoptosis

In some embodiments, the present disclosure describes methods for detecting cell apoptosis, the method comprising: acoustically focusing one or more cells disposed within a fluid; optically exciting the one or more cells with an excitation source; detecting one or more optical signals from the cells; and analyzing the detected optical signals to identify morphological or biochemical changes that are indicative of cell apoptosis. In some embodiments of the method, an optical signal corresponding to detecting an apoptotic event in the cell is indicative of an apoptotic cell and the absence of an optical signal corresponding to detecting an apoptotic event in the cell is indicative of the absence of apoptosis. In some example embodiments a, an optical signal corresponding to detecting an apoptotic event comprises detecting a change in the cells mitochondrial membrane potential, a change in the cells mitochondrial redox potential, a change in the protein composition in the cells plasma membrane and combinations thereof. For example, one illustrative example for detecting apoptosis is detecting an optical signal corresponding to detecting translocation of phosphatidylserine (PS) from the inner leaflet of the plasma membrane of the cell to the outermembrane of the plasma membrane of the cell is indicative of an apoptotic cell.

Apoptosis is a carefully regulated process of cell death that occurs as a normal part of development. Apoptosis is distinguished from necrosis, or accidental cell death, by characteristic morphological and biochemical changes, including compaction and fragmentation of nuclear chromatin, shrinkage of the cytoplasm, and loss of membrane asymmetry. Biochemically, apoptosis is distinguished by fragmentation of the genome and cleavage or degradation of several cellular proteins. As with cell viability, no single parameter fully defines cell death in all systems; therefore, it is often advantageous to use several different approaches when studying apoptosis. The present methods allow detection of apoptosis using flow cytometry. Illustrative methods are demonstrated on the Attune® Acoustic Focusing Cytometer as three apoptotic plasma membrane assays and a mitochondrial membrane potential assay.

Apoptotic plasma membrane assays for flow cytometry: Some of the earliest detectable apoptotic events involve the plasma membrane, including changes in membrane asymmetry and permeability. In addition to annexin V conjugates, Life Technologies provides unique assays to measure membrane changes under conditions where annexin V binding is problematic, such as in adherent cells, and without using special buffers. Annexin V conjugates In apoptotic cells, phosphatidylserine (PS) is translocated from the inner to the outer leaflet of the plasma membrane, thus exposing PS to the external cellular environment. Annexin V labeled with a fluorophore can identify apoptotic cells by binding to PS exposed on the outer leaflet of the membrane. The Alexa Fluor® series of dyes, used in Life Technologies Annexin V Dead Cell Apoptosis Kits provides brighter and more photostable bioconjugates than other organic dyes with similar spectral characteristics. Apoptosis detection with the Annexin V Dead Cell Apoptosis Kit. Jurkat cells (T-cell leukemia, human) treated with 10 μM camptothecin for 4 hr (B) or untreated control (A).

Cells were stained using the Annexin V Dead Cell Apoptosis Kit and analyzed by flow cytometry with 488 nm excitation on the Attune® Acoustic Focusing Cytometer with 530/30 Xnm and 575/24 nm bandpass filters. Data were acquired with a standard 100 XμL/min collection rate. Note that the camptothecin-treated cells (B) have a higher percentage of apoptotic cells than the basal level of apoptosis seen in the control cells (A). A=apoptotic cells, V=viable cells, N=necrotic cells. Monomeric cyanine dyes There are some situations in which staining cells with annexin V is not the optimal method for detecting apoptosis. These include assays where cells are sensitive to the high calcium concentrations required for annexin V binding, assays where phosphatidylserine detection on adherent cells is adversely affected by trypsinization, and assays where washing of samples is prohibitive. Three monomeric cyanine dyes (PO-PRO™-1, YO-PRO®-1, and TO-PRO®-3) (data not shown) have been shown to penetrate apoptotic cells because of permeability changes associated with the loss in asymmetry of the plasma membrane. These dyes enter apoptotic cells and bind to nucleic acids, while cell-impermeant dead cell stains are excluded. The three dyes have unique excitation wavelengths, providing enhanced flexibility in multiplexed assays.

Apoptosis detection using YO-PRO®-1 stain: Jurkat cells (human T-cell leukemia) were treated for 4 hr with 10 μM camptothecin. Cells were then stained with 1.5 μM propidium iodide (PI) and 0.1 μM YO-PRO®-1 and analyzed on the Attune® Acoustic Focusing Cytometer using 488 nm excitation. YO-PRO®-1 was collected with a 530/30 nm bandpass filter and PI was collected with a 575/24 nm bandpass filter. Populations are labeled as L=live cells, A=apoptotic cells, and D=dead cells.

Apoptosis detection using PO-PRO™-1 stain: Jurkat cells (human T-cell leukemia) were treated for 4 hr with 10 μM camptothecin. Cells were then stained with 1.5 μM 7-AAD and 0.1 μM PO-PRO™-1 and analyzed on the Attune® Acoustic Focusing Cytometer using 405 nm and 488 nm excitation. PO-PRO™-1 was collected with a 450/40 nm bandpass filter and 7-AAD was collected with a 640 nm longpass filter. Populations are colored as green=live cells, blue=apoptotic cells, and red=dead cells (data not shown).

Violet Ratiometric Membrane Asymmetry Probe/Dead Cell Apoptosis Kit: The Violet Ratiometric Membrane Asymmetry Probe/Dead Cell Apoptosis Kit provides a simple and fast method for detecting apoptosis with dead-cell discrimination by flow cytometry. The violet ratiometric membrane asymmetry probe F2N12S (4′-N,N-diethylamino-6-(Ndodecyl-N-methyl-N-(3-sulfopropyl))ammoniomethyl-3-hydroxyflavone) is a novel violet diode-excitable dye for the detection of membrane phospholipid asymmetry changes during apoptosis. This dye exhibits an excited-state intramolecular proton transfer (ESIPT) reaction, resulting in dual fluorescence with two emission bands corresponding to 530 nm and 585 nm, and producing a two-color ratiometric response to variations in surface charge. This ratiometric probe is therefore a self-calibrating absolute parameter of apoptotic transformation, independent of probe concentration, cell size and instrument variations.

The Violet Ratiometric Membrane Asymmetry Probe/Dead Cell Apoptosis Kit: Jurkat cells (T-cell leukemia, human) were treated with 10 μM camptothecin for 4 Xhr (B and D) or left untreated (A and C). Cells were stained according to the protocol and analyzed on the Attune® Acoustic Focusing Cytometer. For F2N12S, 405 nm excitation and 522/31 nm and 603/48 nm bandpass filters were used; for SYTOX® AADvanced™ dead cell stain, 488 nm excitation and a 640 nm longpass filter was used. In panels A and B, live cells can be discriminated from apoptotic and dead cells by the relative intensities of the two emission bands from F2N12S. In panels C and D, SYTOX® AADvanced™ dead cell stain fluorescence is plotted against a derived ratio parameter from the two emission bands (585/530 nm) of F2N12S. A=apoptotic cells, L=live cells, D=dead cells.

Mitochondrial JC-1 apoptosis assay for flow cytometry: A distinctive feature of the early stages of apoptosis is the disruption of the mitochondria, including changes in membrane and redox potential. The present disclosure offers a number of fluorescent probes for analyzing mitochondrial activity in live cells by flow cytometry (the MitoProbe™ assays, Life Technologies). Jurkat cells stained with 2 μM JC-1. Cells were stained for 20 min at 37° C. and 5% XCO2, washed with PBS, and analyzed on the Attune® Acoustic Cytometer using 488 Xnm excitation with 530/30 nm bandpass and >640 longpass emission filters. Untreated cultured cells (A)X are shown compared to treated cells (B), which were induced to undergo apoptosis with 10 μM camptothecin for 5 hr at 37° C. The JC-1 dye exhibits potential-dependent accumulation in mitochondria, indicated by a fluorescence emission shift from green (˜529 nm) to red (˜590 Xnm). Consequently, mitochondrial depolarization is indicated by a decrease in the red/green fluorescence intensity ratio, which is dependent only on the membrane potential and not on other factors such as mitochondrial size, shape, and density, which may influence single-component fluorescence measurements.

### A No-Lyse, No-Wash, and No-Cell Loss Method Using the Attune® Acoustic Focusing Cytometer

Analysis of small samples, such as rare samples as well as hard to obtain samples are always a challenge to analyze given sample loss during sample preparation steps during or prior to analysis such as washing, lysing etc. For example, immunophenotyping mouse whole blood presents a challenge due to the limited sample volume available (G100 JL/day/animal) particularly in longitudinal studies. Specifically, these small volumes limit the ability to perform multicolor phenotyping experiments with the required compensation and fluorescence-minus-one (FMO) controls. Methods previously described using a no-lyse, no-wash staining protocol for small sample volumes sacrifice light scatter resolution [Weaver J L, McKinnon K, Germolec D R (2010) Phenotypic analysis using very small volumes of blood. Curr Protoc Cytom 54:6.30.1-6.30.8]. In traditional flow cytometry (which uses hydrodynamic focusing to orient the cells in the flow stream), accurately identifying some cell populations depends on high-resolution scatter data, so no-lyse, no-wash protocols are not feasible on this type of platform. Moreover, the sample dilution required in no-lyse, no-wash methods (to achieve low coincidence with red blood cells and platelets) generally dilutes the cell sample to such an extent that the time required to acquire sufficient events at the flow rates available in those instruments is inordinately long.

Some embodiments of the present disclosure describe a no-lyse, no-wash method using acoustic focusing technology (for example, offered by the Attune® Acoustic Focusing Cytometer). The Attune® cytometer aligns cells in the core stream using acoustic forces that are independent of the fluid stream. This allows a precise alignment of cells in the core and much higher throughput than is possible with traditional flow cytometry. In this application, 5 μL of mouse blood is stained in a 50 μL total volume and then diluted 400-fold in PBS (2 mL final volume). To keep data set sizes manageable, a fluorescence threshold (CD45) is used to distinguish the white blood cell population from the much more abundant red blood cell population. In addition, at this dilution, the coincidence of the target population with red blood cells and platelets is reduced sufficiently so that scatter signals (necessary for accurate differentiation of the granulocyte and lymphocyte populations) are reliable. Acquisition times for such dilute samples are still completed in a reasonable 1-2 minutes, whereas a traditional hydrodynamic focusing cytometer requires 8-10 minutes per sample. This method delivers additional time savings by reducing the number of sample preparation steps and eliminating lysis and wash steps to avoid sample loss.

Materials: •Rat anti-mouse CD45 FITC (Invitrogen Cat. No. MCD4501); •Directly labeled anti-mouse antibodies; •Anti-mouse CD16/32 (Invitrogen Cat.; No. MFCR00); •Attune® Performance Tracking Beads; (Applied Biosystems Cat. No. 4449754); •Mouse peripheral blood; •PBS+1% BSA+2 mM NaN3, pH 7.2; (PBS-BSA); •PBS; •Anticoagulant-coated collection tubes; or 0.5 M EDTA, or 140 USP units/mL; sodium heparin, or 3.8% w/v sodium; citrate; •12×75 mm tubes or other flow; cytometry tubes; •Attune® Acoustic Focusing Cytometer; •AbC™ Anti-Rat/Hamster Bead Kit; (Invitrogen Cat. No. A10389).

Titration of antibodies: Titrate all antibody conjugates using the following staining protocol to determine optimal staining concentration. Antibody conjugates may be used at the manufacturer's recommended staining concentration with the AbC™ Anti-Rat/Hamster Bead Kit. For multicolor testing, premix antibody conjugates in 1×PBS-BSA to provide the final antibody mixture in a 45 μL total volume.

Prepare blood: 1. Collect peripheral blood following Institutional Animal Care and Use Committee (IACUC) acceptable practices. If an anticoagulant-coated collection device is not used, then add 1/10 the volume of anticoagulant (0.5 M EDTA, or 3.8% w/v sodium citrate, or 140 USP units/mL sodium heparin) to the whole blood and mix well. 2. Block Fc binding receptors by pretreating with 0.1 μg of CD16/CD32 per 10 μL of whole blood and incubating for a minimum of 10 minutes prior to antibody labeling.

Staining protocol: 1. Pipet antibody conjugates into labeled sample tubes; the volume should be 45 μL. 2. Add 5 μL of anticoagulated mouse whole blood to the antibody solution and mix well. 3. Incubate protected from light for 30 minutes (or reagent manufacturer's recommendation). 4. Add 2 mL PBS to the tubes immediately prior to loading on the Attune® Acoustic Focusing Cytometer.

Compensation controls: Prepare single-color compensation samples using the AbC™ Anti-Rat/Hamster Bead Kit. 1. Add one drop of AbC™ Anti-Rat/Hamster Capture Beads (Component A) to a labeled sample tube for each antibody conjugate included in the panel. 2. Add the recommended amount of each rat or hamster antibody conjugate to the AbC™ Anti-Rat/Hamster Capture Beads. 3. Incubate for 15 minutes at room temperature, protected from light. 4. Add 3 mL PBS and centrifuge for 5 minutes at 200×g. 5. Carefully remove the supernatant and resuspend the bead pellet by adding 1 mL PBS. 6. Prepare one AbC™ Anti-Rat/Hamster Control Beads (Component B) sample by adding 1 drop to a labeled sample tube along with 1 mL PBS.

Verify instrument performance with the Attune® Performance Tracking Beads: 1. Create a new experiment using instrument settings optimized for no-lyse, no-wash staining. 2. In the experiment browser, rightclick Compensation and select Compensation setup. 3. In the Compensation setup dialog box, select to compensate on height and select the parameters required for the selected panel. Note: Pulse height is used for both scatter and fluorescence signals, as it provides lower measurement standard deviations than pulse area. 4. There should be one single-color compensation sample matched to each parameter selected. 5. Click OK to create the compensation control samples. 6. Select the workspace for the specimen sample by double-clicking on the first tube for the specimen in the experiment browser. 7. Create a bivariate plot of the CD45+ threshold channel vs. SSC and additional plots as needed for data analysis. 8. Place a CD45+ polygon gate around the CD45-positive population (these steps were performed and results are not expressly shown herein). 9. Optimize the fluorescence threshold to include all of the lymphocyte and granulocyte populations. 10. Optimize the remaining fluorescence PMT voltage levels. 11. Once instrument optimizations are complete, select the first compensation control tube. 12. Enable forward scatter threshold for the compensation controls by changing the FSC threshold logic to “or”. 13. Beads require a forward scatter threshold. One may also need to adjust the forward and side scatter voltages to place the bead singlets on scale. There is no need to change fluorescence PMT voltages from those optimized for cells being tested. Compensation controls and the stained cell panel must be run at the same fluorescence voltages to obtain a valid compensation matrix. 14. Set the recording conditions to collect 5,000 R1 events, acquisition volume to 200 μL, and flow rate to 200 μL/min. 15. Run the Component B compensation control, adjust the R1 gate to include only bead singlets, and record the data. 16. Copy the R1 gate to the remaining compensation controls and run each tube in order. 17. After completing the compensation controls, return the FSC and SSC PMT voltages to values optimized for cells (if changed) and reset the FSC threshold logic back to ‘Ignore’ to threshold the whole blood only on fluorescence. 18. Select a first sample tube of stained cells. 19. Set the collection rate to 500 μL/min acquisition volume and recording criteria to obtain the desired events (recommend starting with 600 μL volume for 10,000 CD45+ events). 20. Proceed with collecting data for samples. CD45 FITC BL1oH SSCoH p106q

A density plot analysis of 5-color panel with (A) a daughter plot of CD45+ gate showing the B cells stained with rat anti-mouse CD45R Pacific Blue™ conjugate (parameter VL1-H, violet laser, filter 450/40 nm BP) and T cells stained with hamster anti-mouse CD3 PE-Cy®5 (parameter BL3-H, blue laser, filter 530/30 nm BP) was obtained (data not shown. A second (B) density dot plot, a daughter of the CD3−CD45R− gate, showing the double-positive granulocytes stained with rat antimouse CD11b PE (parameter BL2-H, blue laser, 575/24 nm BP filter) and rat anti-mouse GR-1 Pacific Orange™ conjugate (parameter VL3-H, violet laser, 603/48 nm BP filter) was also obtained (data not shown). The CD11b+GR1− population represents phagocytes (monocytes, macrophages, and any circulating dendritic cells).

Data analysis: 1. Gate on events expressing CD45 in a CD45 FITC vs. SSC dot plot. 2. Derive daughter plots from CD45+ gating to analyze the target populations. 5-color example In the following example, whole mouse (BALB/CJ) blood was stained with a 5-color panel comprising rat anti-mouse CD45 FITC (pan-leukocyte), rat anti-mouse CD11b PE (monocytes, granulocytes, macrophages, dendritic), hamster anti-mouse CD3e PE-Cy®5 (T cells), rat anti-mouse CD45R Pacific Blue™ (B220, B cells), and rat anti-mouse GR-1 (Ly-6C/G, granulocytes) conjugates. Titer for these five direct conjugates ranged from 0.008 μg to 0.125 μg per test. This five color staining example required five FMO control samples and one 5-color panel sample tube. The FMO controls were used to both fine-tune compensation levels and determine appropriate quadrant marker placement for the panel. Two density plots were created to identify the four subpopulations—B lymphocytes, T lymphocytes, monocytes, and granulocytes—from the 5-color panel. First, a daughter plot is created from the CD45+ gate for CD3 PE-Cy®5 vs. CD45R Pacific Blue™ conjugate (data not shown). Quadrant markers are set from the FMO controls minus the CD3 PE-Cy®5 dye and minus CD45R Pacific Blue™ dye to identify the CD45R+ (B lymphocyte) and CD3e+ (T lymphocyte) populations. Next, a second daughter density plot is created from the negative cell population CD3−CD45R− (non-B, non-T cells) to identify the granulocyte and monocyte population expression of GR-1 and CD11b.

Preparing antibodies: Successful results with this no-lyse, nowash method depend on testing titrated, directly conjugated antibodies with this staining method prior to preparing mixed antibody cocktails. Indirect staining is not compatible with the no-wash staining protocol, and only the use of directly labeled or Zenon® labeled antibodies is recommended. Antibody conjugates must be titered in a single color following this method, and optimal titer selected where there is maximal separation of positive cells with minimal background of the negative population. The use of antibody capture beads as a compensation control is recommended. This saves valuable sample in cases where the targeted population is rare for the marker requiring compensation. It is not necessary to titrate the conjugated antibodies on the AbC™ Anti-Rat/Hamster Beads separately from the mouse blood titration. Conjugated antibodies may be used per manufacturer's recommended amount for cell staining. Reagent consumption may be minimized by titrating conjugates antibodies on AbC™ Anti-Rat/Hamster Beads. The correct titer will provide a fluorescence median intensity of the AbC™ Anti-Rat/Hamster Beads at least as bright as the stained whole blood. Do not expect the titer determined for staining mouse blood to be appropriate for staining AbC™ Anti-Rat/Hamster Beads. Though the current example uses CD45 as the threshold to select all white blood cells, it may be more practical to use the major cell type included in a panel as the threshold parameter. For example, using a CD3 threshold for T cells or CD19 for B cells rather than CD45 would save an additional parameter for subtyping in multicolor experiments. When selecting a dye as a threshold, it is recommended to use one such as FITC, Alexa Fluor® 488 dye, or Pacific Blue™ dye, which have minimal spillover from other dyes into their channels.

### Computer Software Programs and Methods

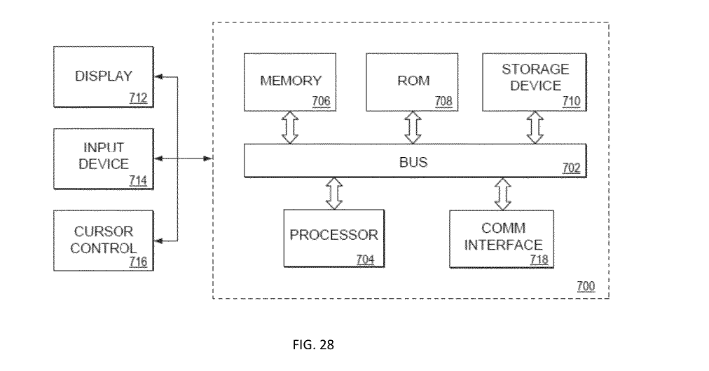

One or more methods of the disclosure described herein may be performed using a computer system having a computer program comprising a non-transitory computer-readable storage medium encoded with instructions, executable by a processor, the instructions comprising instructions for performing a method for analyzing a labeled bioparticle. FIG. 28 is a block diagram that illustrates a computer system 700 that may be employed to carry out processing functionality, according to various embodiments. Computing system 700 can include one or more processors, such as a processor 704. Processor 704 can be implemented using a general or special purpose processing engine such as, for example, a microprocessor, controller or other control logic. In this example, processor 704 is connected to a bus 702 or other communication medium. It should be appreciated that a computing system 700 of FIG. 28 may be embodied in any of a number of forms, such as a rack-mounted computer, mainframe, supercomputer, server, client, a desktop computer, a laptop computer, a tablet computer, hand-held computing device (e.g., PDA, cell phone, smart phone, palmtop, etc.), cluster grid, netbook, embedded systems, or any other type of special or general purpose computing device as may be desirable or appropriate for a given application or environment. Computer system 700 can be functionally linked to an acoustic cytometer or may be comprised in an acoustic cytometer. Additionally, a computing system 700 can include a conventional network system including a client/server environment and one or more database servers, or integration with LIS/LIMS infrastructure. A number of conventional network systems, including a local area network (LAN) or a wide area network (WAN), and including wireless and/or wired components, are known in the art. Additionally, client/server environments, database servers, and networks are well documented in the art.

Computing system 700 may include bus 702 or other communication mechanism for communicating information, and processor 704 coupled with bus 702 for processing information.

Computing system 700 also includes a memory 706, which can be a random access memory (RAM) or other dynamic memory, coupled to bus 702 for storing instructions to be executed by processor 704. Memory 706 also may be used for storing temporary variables or other intermediate information during execution of instructions to be executed by processor 704. Computing system 700 further includes a read only memory (ROM) 708 or other static storage device coupled to bus 702 for storing static information and instructions for processor 704.

Computing system 700 may also include a storage device 710, such as a magnetic disk, optical disk, or solid state drive (SSD) is provided and coupled to bus 702 for storing information and instructions. Storage device 710 may include a media drive and a removable storage interface. A media drive may include a drive or other mechanism to support fixed or removable storage media, such as a hard disk drive, a floppy disk drive, a magnetic tape drive, an optical disk drive, a CD or DVD drive (R or RW), flash drive, or other removable or fixed media drive. As these examples illustrate, the storage media may include a computer-readable storage medium having stored therein particular computer software, instructions, or data.

In alternative embodiments, storage device 710 may include other similar instrumentalities for allowing computer programs or other instructions or data to be loaded into computing system 700. Such instrumentalities may include, for example, a removable storage unit and an interface, such as a program cartridge and cartridge interface, a removable memory (for example, a flash memory or other removable memory module) and memory slot, and other removable storage units and interfaces that allow software and data to be transferred from the storage device 710 to computing system 700.

Computing system 700 can also include a communications interface 718. Communications interface 718 can be used to allow software and data to be transferred between computing system 700 and external devices such as an acoustic cytometer or an acoustic cytometery apparatus/system. Examples of communications interface 718 can include a modem, a network interface (such as an Ethernet or other NIC card), a communications port (such as for example, a USB port, a RS-232C serial port), a PCMCIA slot and card, Bluetooth, etc. Software and data transferred via communications interface 718 are in the form of signals which can be electronic, electromagnetic, optical or other signals capable of being received by communications interface 718. These signals may be transmitted and received by communications interface 718 via a channel such as a wireless medium, wire or cable, fiber optics, or other communications medium. Some examples of a channel include a phone line, a cellular phone link, an RF link, a network interface, a local or wide area network, and other communications channels.

Computing system 700 may be coupled via bus 702 to a display 712, such as a cathode ray tube (CRT) or liquid crystal display (LCD), for displaying information to a computer user. An input device 714, including alphanumeric and other keys, is coupled to bus 702 for communicating information and command selections to processor 704, for example. An input device may also be a display, such as an LCD display, configured with touchscreen input capabilities. Another type of user input device is cursor control 716, such as a mouse, a trackball or cursor direction keys for communicating direction information and command selections to processor 704 and for controlling cursor movement on display 712. This input device typically has two degrees of freedom in two axes, a first axis (e.g., x) and a second axis (e.g., y), that allows the device to specify positions in a plane. A computing system 700 provides data processing and provides a level of confidence for such data. Consistent with certain implementations of embodiments of the present teachings, data processing and confidence values are provided by computing system 700 in response to processor 704 executing one or more sequences of one or more instructions contained in memory 706. Such instructions may be read into memory 706 from another computer-readable medium, such as storage device 710. Execution of the sequences of instructions contained in memory 706 causes processor 704 to perform the process states described herein. Alternatively hard-wired circuitry may be used in place of or in combination with software instructions to implement embodiments of the present teachings. Thus implementations of embodiments of the present teachings are not limited to any specific combination of hardware circuitry and software.

The term “computer-readable medium” and “computer program product” as used herein generally refers to any media that is involved in providing one or more sequences or one or more instructions to processor 704 for execution. Such instructions, generally referred to as “computer program code” (which may be grouped in the form of computer programs or other groupings), when executed, enable the computing system 700 to perform features or functions of embodiments of the present disclosure. These and other forms of computer-readable media may take many forms, including but not limited to, non-volatile media, volatile media, and transmission media. Non-volatile media includes, for example, solid state, optical or magnetic disks, such as storage device 710. Volatile media includes dynamic memory, such as memory 706. Transmission media includes coaxial cables, copper wire, and fiber optics, including the wires that comprise bus 702.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 704 for execution. For example, the instructions may initially be carried on magnetic disk of a remote computer. The remote computer can load the instructions into its dynamic memory and send the instructions over a telephone line using a modem. A modem local to computing system 700 can receive the data on the telephone line and use an infra-red transmitter to convert the data to an infra-red signal. An infra-red detector coupled to bus 702 can receive the data carried in the infra-red signal and place the data on bus 702. Bus 702 carries the data to memory 706, from which processor 704 retrieves and executes the instructions. The instructions received by memory 706 may optionally be stored on storage device 710 either before or after execution by processor 704.

It will be appreciated that, for clarity purposes, the above description has described embodiments of the disclosure with reference to different functional units and processors. However, it will be apparent that any suitable distribution of functionality between different functional units, processors or domains may be used without detracting from various embodiments of this disclosure. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

In some embodiments of the present disclosure, methods are described for analyzing a bioparticle that may be performed (executed), by a user, to obtain data regarding the bioparticle analyzed, comprising one or more steps (e.g., a workflow or in some embodiments multiple workflows to obtain a pipeline of workflows) that may be accessible and controllable by the user via a Graphical User Interface (GUI) that is visible on Display 712. A user may enter data (e.g external data) and/or select options provided in the GUI using Input Device 714 and/or Cursor Control 716. In some embodiments, components of computer system 700 convert input data provided by a user into a computer readable format to one or more computer system components (such as a memory, a database, a processor etc.) to enable interpretation of input data received from a user and to initiate controller instructions to conduct one or more steps of the acoustic flow cytometry method.

In some embodiments, user input data may also be used for report generation of the particular method being performed. In some embodiments, components of computer system 700, such as Display 712, may also receive data from one or more processors/sensors/detectors following performing one or more steps of a method that are then converted into a user understood format to enable a user to monitor progress of the workflow steps and/or to obtain additional input from a user to determine the next course/step of the workflow in a method of the disclosure. Input of data from a user or translation of data received from various devices within computer system 700 may be mediated by components of a software (or computer program) of the disclosure (not expressly depicted) which comprises comprising a computer readable medium comprising computer readable instructions, which, when executed by the computer system, are configured to display on Display 712 (screen, LCD).

A software (or computer program) of the disclosure may be operable to receive user instructions, either in the form of user input into a set parameter fields, e.g., in a GUI, or in the form of pre-programmed instructions such as but not limited to pre-programmed instructions for performing a variety of different specific operations and/or for analyzing various parameters and/or for analyzing one or more data components (optical signal data). A software of the disclosure, in some embodiments, may be operable to convert pre-programmed instructions to appropriate computer language for instructing operation of system 700 to carry out a desired operation. A software of the disclosure, in some embodiments, may be operable to convert data signals or parameters received into appropriate computer language that may then be analyzed by a processor in computer and/or converted into user viewable format for a user to review or analyze.

In some embodiments, a software of the disclosure may comprise functional specifications as well as graphical user interface (GUI) specifications. GUI specifications enable user mediated methods. Exemplary GUI's of the present disclosure may comprise some general GUI specifications. In some embodiments, general GUI specifications may comprise all screens, with the exception of pop-up screens, being 800 pixels wide and 480 pixels high.

Other general GUI specifications may include without limitation, the availability of a Home button in all menu screens where Home button allows a user to navigate to a Main Menu; the availability of Breadcrumbs or a Breadcrumb Trail in all menu screens (breadcrumbs may be abbreviated when they are too long for display); the availability of Time and Date in all menu screens; the availability of a Back button in all menu screens where a Back button allows a user to navigate to a previous screen; the availability of a Save button in screens where a user can change and save one or more fields. Breadcrumbs refer to a navigation aid used in a user interface to show the path that a user has taken to arrive at a screen.

In some embodiments, in a screen where a Save button is available, a Back button may allow a user to either save or cancel a change, if any, before navigating to previous screen. In some embodiments, in a screen where a Save button is available, a Home button allows a user to either save or cancel a change, if any, before navigating to a Home screen. General GUI specifications also include the availability of a Keypad in screens where a user needs to enter an alpha-numeric string or special character keys.

### FURTHER EXAMPLES

The disclosure is further illustrated by the following non-limiting examples.

Probes that Particularly Benefit from Longer Transit Time

Probes useful in accordance with the present disclosure and that are uniquely enabled by the present disclosure, include but are not limited to the following:

Dimmer Labels

Extinction coefficient less than 25,000 cm⁻¹M⁻¹ e.g. Alexa 405 and 430 and or quantum efficiency less than 25% including but not limited to ruthenium, and Cy3.

Photobleach susceptible or triplet state prone dyes.

Lower Laser Power

Dyes that suffer from photobleaching including but not limited to blue fluorescent protein or triplet state quenching including but not limited to PerCP from medium power laser spots (>10,000 W/cm²).

For ultra-high power laser (>50,000 W/cm², as often used in stream in air sorters). This utility expands to include most dyes including Phycoerythrin and fluorescein.

Longer Lifetime (Continuous Wave Excitation)

Lifetimes greater than 10 nanoseconds including Q-dots, Q-dot tandems, lanthanides, lanthanide tandem dyes, transition metal ligand complexes and phosphor particles. The longer transit time allows more cycles of excitation and emission, resulting in more overall photons emitted.

Longer Lifetime (Pulsed or Modulated Excitation)

The same class of probes as for longer lifetime CW excitation benefit but there is particular utility for luminescent probes including but not limited to lanthanides/lanthanide tandems and phosphors that have lifetimes in excess of the transit time in conventional cytometry (>10 microseconds).

Bioluminescent, Chemiluminescent

Any probe that produces light from a chemical process (including photoactivated processes) that takes longer to emit the majority of photons in a conventional cytometry transit time (>10 microseconds) including but not limited to luciferin/luciferase, Ca⁺²/aequorin.

Radioactive Probes/Scintillation

For extremely slow transit>100 milliseconds per particle. These types of particles are not analyzed in cytometry (even slow flow) due to the long integration times necessary to gather enough photons.

Probes Resistant to Photobleaching with High Laser Power

Using very high laser power (>50,000/cm²) combined with photobleaching resistant probes including but not limited to dye loaded nanospheres, O-dots® or C-dots® and very tight spatial filtering (as in confocal microscopy) high signal to noise ratio can be achieved. Long transit times of greater than 10 microseconds combined with a very tight acoustically focused “core diameter”, the spatial filtering achieves superior signal to noise.

Dimmer Labels

Dimmer labels, i.e. those having low extinction coefficients and or low quantum yields will yield more photons with longer excitation times. Some dyes may have particular utility in certain applications including but not limited to UV excited dyes for multi-color analysis including but not limited to Alexa 405 and 430 or relatively dim long Stokes shift dyes including but not limited to APC-C7. Other dyes may offer more opportunity for developing assaying that normally use brighter labels including but not limited to using dimmer tandem dyes vs. phycoerythrin tandem dyes.

Also included in the category of dimmer labels are naturally occurring fluorescent species including but not limited to NAD(P)H. Some applications for monitoring of such low quantum yield species and slower transit time systems can make this easier with higher sensitivity and more importantly, greater fluorescence resolution between cells or particles.

Less Photostable Dyes

Lower laser power and longer transit time can increase the overall output such that dyes, including but not limited to fluorescent protein or BFP could become more useful in flow.

Probes Prone to Non-Radiative State Excitation

Fluorophores as diverse as Rhodamine Atto532 and green fluorescent protein or GFP can give off many more fluorescent photons before destruction when allowed to relax from long lived triplet states following intense laser pulses. There is greater emission for pulses with longer dead time corresponding to the dark time estimated for triplet states (˜1 microsecond). This emission is also significantly greater than for the equivalent power of continuous wave excitation. In conventional flow, one could not use a system with such long dead times between pulses, particularly for probes requiring longer relaxation times such as PerCP (˜7 microsecond triplet state lifetime). The present disclosure allows for these longer relaxation times.

Quantum Dots

With quantum dots, their long fluorescence lifetimes reduce the amount of light they can give off when their excitation time is limited. In a longer transit, field focused system however, as in the present disclosure this limitation does not exist. The long transit times can elicit very bright signals even from just a few Q dots. Another problem with Q dots is that they are made using toxic materials creating large volumes of potentially hazardous waste. With the present disclosure, fewer Q dots can be utilized and waste volumes in field focused systems are typically ˜100-1000 times smaller.

Luminescent Probes

In general, any long lifetime probe will not emit photons as quickly as a shorter lifetime probe so the performance of nearly all such probes can be tremendously improved with longer transit times as more photons per label can be emitted. Also, lanthanides can be loaded at very high concentrations in nanoparticles due to the fact that they do not self-quench easily. This allows particle tags to be much brighter than tags in solution.

Lanthanides have fluorescent lifetimes on the order of microseconds to milliseconds with the most common europium chelates having a lifetime around 0.7 milliseconds. Such probes are used for extremely sensitive assaying in which background fluorescence is gated out in time from the luminescent signal by pulsing the light source and waiting to collect light the background fluorescence has decayed. This type of luminescent assaying are useful in the field focused, long transit time system of the present disclosure. Tandem lanthanide fluorophores or assaying that use lanthanide based energy transfer to conventional fluorophores such as the europium/allophycocyanin based TRACE™ system (Perkin-Elmer, Waltham, Mass.) and the terbium based Lanthascreen™ (Invitrogen, Carlsbad, Calif.) can also be implemented effectively in the present disclosure. The lifetimes are shorter when energy transfer to the other fluorophore is possible. The lifetimes are much too long to be practical in conventional flow cytometry.

Medium switching can be applied to luminescent reactions for which exposure to luminescence reagents is controlled in time. In addition, serial or parallel reactions designed for permeation or lysis of membranes in order to facilitate diffusion of chemi or bioluminescent species can be implemented with precise timing and nearly equivalent exposure of cells to reagents. For example, cells expressing luciferase can be transferred into a medium containing both lysing/permeation reagent and luciferin. As the membrane becomes permeable to the luciferin substrate the cell will begin luminesceing.

This process can of course be extended to other procedures using cell permeation such as gene transfection or cell loading of other membrane impermeant molecules/constructs. This in-line permeation can be carefully controlled with regards to time exposure of cells to lysis reagents by transferring cells in and out of the reagents sequentially.

Absorptive Dyes/Axial Light/Less Absorptive Probes

Non-fluorescent absorptive dyes are used very commonly in microscopy but not in flow cytometry due to small signal to background. With increased transit time, integration is possible such that the signal to noise can be greatly increased. Additionally, advances in high speed linear array detectors make it possible to increase signal by spatial isolation of the axial (approximately 0 degrees relative to the excitation source) light loss. Such arrays can scan fast enough to suit a slow transit time system and can give information regarding not only axial light loss but several angles of light scatter.

With a slower transit time system, bead arrays using different color and concentration of absorptive dyes can be made practical for multiplex assaying. For multiplexing with more than one color, two lasers of different color are required such that differential color absorption can be observed. If the lasers are collocated, the detectors need to be made color sensitive unless the excitation is separated in time. One advantage of such arrays is that the absorptive dyes do not interfere significantly with fluorescence tagging. Alternatively, one laser can be used if absorption is combined with another parameter such as fluorescence. Still another embodiment uses absorptive dyes with a wide band excitation source such as an LED and at least two color sensitive detectors.

With much slower linear velocities (cells can even be stopped momentarily) imaging in flow for Pap smears becomes much easier such that both morphology and the information from absorptive dyes can be collected on a field focused system.

Radioactive Tracers

Radioactive labels are useful in the present disclosure due to the need for long exposure times. The exposure required is on the order of seconds. In particular, pharmaceutical screening assaying that use small molecule species or other species where a fluorescent tag might interfere with the specific action of the pharmaceutical candidate being tested can benefit from the present disclosure.

Bioluminescent and Chemiluminescent Probes

These probes can be extremely sensitive as their signal is created without background producing excitation light. The signal from such probes is integrated over times periods from 10 microseconds to 10 seconds or more, much longer than conventional flow transit times.

Raman Scattering Probes

The field-focused systems of the present disclosure allow signal integration and averaging of noise such that detection of Raman signals is possible.

### Example 2

Acoustic washing of the sample can eliminate most or nearly all centrifugation steps in flow assaying (or other types of assaying that would benefit from sample washing prior to analysis).

This not only saves tremendous amounts of technician time, but it also automates a tedious process that is prone to operator error particularly due to fatigue. It also reduces exposure of operator to potentially bio-hazardous materials.

Adjustable Concentrator/Washer

Acoustic concentration and washing can replace centrifugation operations for many assaying methods. It has many advantages including extremely clean and gentle separation and reduced operator variation. In addition it presents new opportunities for sample processing that cannot be achieved in conventional centrifugation. By adjusting the concentration ratios used in an acoustic washer, one can chose the output concentration of a sample. If for example, the sample's initial concentration is 105 particles per milliliter and the operator desires a final concentration of 106 particles per milliliter, the operator can select flow rates for the sample and collection channels that achieve a 10 fold concentration. This process can be automated such that the user need only enter a concentration factor. It can be further automated by adding a spectrometer on or off-line from the separator that determines initial sample concentration based on light scatter and calculates the necessary flow rates to achieve a concentration given by the operator. If a sample is too dilute for the separator to accomplish the desired concentration, it can also perform several concentrations in series. If for example the starting concentration is 103 particles per milliliter, the 10 fold concentration can be performed 3 times to achieve the desired concentration.

Immunophenotyping Wash

Depending on the protocol used, sample prep may include just one separation with one device or it might include a series of steps to automate a more complex protocol. FIG. 8 is a schematic of one embodiment of the present disclosure. In an automated system, cells and labels are added together and incubated initially. Then various steps including washing, lysis, fixation and concentration can all be accomplished using acoustic modules without centrifugation. For example, a first step includes the transfer of blood cells from serum to eliminate serum proteins and the wash medium can contain red cell lysing reagents. The next step includes transferring the remaining cells into a quench medium to stop lysis. This medium could contain staining antibodies or the cells could be concentrated into an incubation chamber where antibodies are added. After incubation cells would then be sent to analysis where they are washed in-line to eliminate background from unbound antibodies.

It is possible to add additional processes and or washing modules in order to automate further steps, e.g. adding more reagents, and/or provide for extra washes if necessary. The acoustic cytometer is coupled to the detector and can also be fitted with an in-line medium switcher if desired.

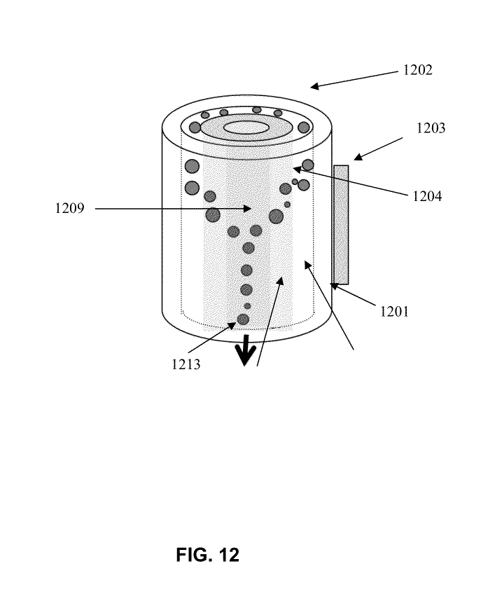

While FIG. 8 illustrates a serial process using more than one separator, it is also possible to do “parallel” media changes in which more than one wash or reaction medium is flowed into a single separator and particles or cells pass sequentially through the media lamina as they move toward the center (FIG. 12). This concept can be extended to include a continuous gradient type of fractionation in which several fluids of incremental contrast are simultaneously injected into the separator. One can envision several complex protocols automated by adding more and more serial or parallel media changes or combinations thereof.

FIG. 12 illustrates a schematic of parallel medium switching device. Multiple media can be used in laminar layers. Sample 1205 is added to capillary 1201. Sample 1205 contains a first particle 1204 and a second particle 1202. A second medium is added to capillary 1201. A third medium 1209 is added to capillary 1201. A line drive 1203 induces acoustic wave and first fluid, second fluid, third fluid, first particle and second particle are acoustically focused/reoriented based upon the acoustic contrast of each. Acoustically focused particles flow out of the capillary 1213. The third fluid is preferably introduced into a channel, the third fluid having a third acoustic contrast relative to the first fluid and the second fluid. The third fluid may contain particles, and the third fluid preferably moves in a third laminar flow stream. The third fluid can have an acoustic contrast that is greater than, lesser than, or the same as the acoustic contrast of the second fluid. The third stream can also be acoustically reoriented based upon the acoustic contrast of the third fluid. A portion of particles that may be in the first fluid can be acoustically focused from the first fluid to the third fluid. This portion of particles preferably passes through the second fluid, wherein the second fluid is preferably a reagent stream. A portion of particles may also be acoustically focused from the second fluid to the third fluid.

Immunophenotyping Panels

In clinical immunophenotyping laboratories, assaying is often done on a single patient's blood in order to classify a particular disorder. The amount of assaying can be reduced by increasing the number of markers that can be assayed at once. Assaying is mostly performed with no more than 4 antibodies because of overlapping spectra for fluorescent tags. Controls for compensation in which each assaying is run without one of the four antibodies greatly increase the amount of assaying that must be performed and add a huge burden in terms of technician time, reagent consumption and analysis time. Performing the current panels without need for compensation promises to greatly streamline the process and performing larger compensation free panels of, for example 6 or more antibodies at once, can reduce assaying significantly.

Compensation is simpler for assaying with fewer colors but they can also benefit from a compensation free panel of antibodies. A very common example is a panel of anti-CD45, CD4, and CD8 antibodies which is used for CD4 positive enumeration of T-cells in AIDS progression monitoring. CD3 is often added or substituted in this panel to aid with identification of T-cells.

Table 1 below is an example list of assaying for four colors done for new patient classification of leukemia/lymphoma. This screen is used for diagnosis of new patients where the disease classification is not known. The four cell markers are listed on the left and the utility of assaying is listed at right. Typically analysis is done on a blue (488 nm) and red (635 nm) laser cytometer with each antibody having a different fluorochrome. A very common combination is FITC, PE, PE-Cy5® and APC. In an acoustic cytometer equipped with long lifetime analysis capabilities, one or more probes can be replaced with a long lifetime probe for which overlapping spectral signal can be subtracted based on temporal measurements. Long transit time can also be used to make up for lost photons if fluorescence filters are narrowed to prevent overlapping spectra. In a system equipped with a violet laser and a blue channel that is not otherwise used to detect a short-lifetime probe, auto-compensation for autofluorescence can be done on a cell by cell basis.

TABLE 1
Table 1:

| | |
|---|---|
| CD3 | This antibody combination is designed to give a differential. CD45 vs SSC is used to define |
| CD14 | lymphocytes, monocytes and granulocytes. CD14 further defines monocytes while CD3 |
| HLADr | gives T-cells and HLADr identifies B-cells and NK cells. In bone marrow, progenitor cells |
| CD45 | are CD4 dim HLADr+ and erythroid cells or platelets are CD45 negative. |
| CD7 | This combination is used for three reasons. 1) Normal T-cells express CD2 and CD7, which |
| CD13 | are often expressed at abnormal levels by malignant T-cells. NK cell malignancy is usually |
| CD2 | CD7+CD2−. 2) CD2+CD7+CD13+ cells represent aberrant co expression of the myeloid |
| CD19 | antigen, CD13, on acute T lineage lymphocytic leukemias and lymphoproliferative disorders. |
| | 3) Co expression of CD2 or CD7 or CD13 with CD19 defines aberrant expression of these |
| | markers on B lineage malignancy. Finally, CD19 is often co-expressed on CD13 AML- |
| | FAB/M2 with a t(8,21) translocation. |
| CD5 | This combination is designed to resolve B-lineage lymphoproliferative disorders. Clonal |
| Lambda | excess of kappa or lambda on CD19 positive cells or CD19CD5 positive cells are explicitly |
| CD19 | defined. |
| Kappa | |
| CD20 | This combination is used to further characterize B-lymphoproliferative disorders and to |
| CD11c | define the degree of maturity of acute B-lineage leukemias. In addition, hairy cell leukemia |
| CD22 | can be classified by its unique high expression of CD11c. Aberrant expression of the T-cell |
| CD25 | marker CD25 on B-cells is diagnostic for lymphoproliferative diseases when it is expressed. |
| CD5 | This antibody combination is designed to resolve the cells within the maturation of both T |
| CD19 | and B-cell lineages. The earliest T-cells are CD5+CD10+CD34+, which differentiate into |
| CD10 | CD5+CD10+ by losing CD34 and finally into mature T-cells that express only CD5. This |
| CD34 | combination can be used to evaluate the maturity of a T lineage acute leukemia. In a like |
| | manner, the maturation of the two distinct B-cell lineages: CD19+CD5− and CD19+CD5+ |
| | can be defined. The earliest B-cells co express both CD34 and CD10. As maturation |
| | occurs, they lose CD34, then CD10 to become mature B-cells. |
| CD15 | This combination is used to define aberrant antibody expression on hematopoietic |
| CD56 | malignancies. CD56 is expressed early on progenitor cells (CD34+CD56+) that may also |
| CD19 | co-express CD15. The present inventors have shown that in acute leukemia, co-expression |
| CD34 | of CD15, CD56 and CD34 is associated with the t(8,21) translocation, which results in a |
| | very bad prognosis. CD15 is also expressed on granulocytes and some B-cells. |

Table 2 below illustrates examples of assaying six color leukemia/lymphoma cells that utilize six labels to reduce the amount of assaying that must be run. Each assaying is numbered on the left, the top column is the fluorochrome used for each antibody and the specificity of each antibody is listed left to right underneath its respective fluorochrome label. In this table there is significant spectral overlap. Again by replacing fluorochromes with a long-lifetime reagents and narrow band reagents, minimal compensation antibody panels are possible.

TABLE 2
Table 2:

| | FITC | PE | PerCP-CY5.5 | PE-CY7 | APC | APC-CY7 |
|---|---|---|---|---|---|---|
| 1 | CD7 | CD4 | CD2 | CD8 | CD3 | CD45 |
| 1. | Kappa | Lambda | CD5 | CD10 | CD34 | CD19 |
| 2. | CD38 | CD11c | CD22 | CD19 | CD23 | CD20 |
| 3. | CD57 | CD56 | CD33 | CD8 | CD161 | CD3 |
| 4. | CD11b | CD13 | CD33 | HLADr | CD34 | CD45 |
| 5. | CD71 | CD32 | CD41a | CD16 | CD64 | CD45 |

Table 3 below shows an example of labels that accomplish compensation minimized results that do not require compensation controls. The instrument uses 405 nm and 635 nm pulsed diode lasers.

TABLE 3
Table 3:

| | Qdot ®545 | Qdot ®800 | EuropiumDEADIT | PerCP | APC | AlexaFluor ®405 |
|---|---|---|---|---|---|---|
| 1. | CD7 | CD4 | CD2 | CD8 | CD3 | CD45 |
| 1. | Kappa | Lambda | CD5 | CD10 | CD34 | CD19 |
| 2. | CD38 | CD11c | CD22 | CD19 | CD23 | CD20 |
| 3. | CD57 | CD56 | CD33 | CD8 | CD181 | CD3 |
| 4. | CD11b | CD13 | CD33 | HLADr | CD34 | CD45 |
| 5. | CD71 | CD32 | CD41a | CD18 | CD64 | CD45 |

Temperature

Temperature can also affect specific gravity and therefore acoustic contrast. This feature can be manipulated by pre-cooling and/or pre-heating one or more of the input streams or by heating or cooling different parts of the separator so as to create a temperature gradient in the fluid stream.

### Example 3

### In-Line Red Cell/Cell Lysis

By incorporating a rapid red cell lysis reagent into the central wash stream, it is possible to lyse red cells in-line in a flowing separator. After lysis, the unlysed white cells can be quickly transferred to a quenching buffer in a subsequent separator. This operation can be performed in seconds, minimizing damage or loss of white cells. The second operation can also be used to exclude debris including lysed red cell “ghosts” that have decreased acoustic contrast resulting from the lysis process.

Concentration of Analytes/White Cells to Decrease Labels or Time

Often, staining of white blood cells for immunophenotyping is done in a small volume of blood prior to lysis. Alternatively, staining can be done after lysis but the sample volume and number of white cells must be carefully controlled in order to insure the proper immune-reaction. The acoustic wash system can be used to concentrate target cells or particles to a small volume for proper immunostaining. This feature is particularly valuable for samples with a low concentration of target cells as it allows a smaller staining volume and therefore less antibody. For example, such a system can be used to decrease the cost of assaying in CD 4+ T cell counting for AIDS progression monitoring. It is also valuable for removal of native serum antibodies that might interfere with proper white cell staining, particularly when coupled with acoustic washing. It is also valuable for removal of native serum antibodies that might interfere with proper white blood cell staining, particularly when coupled with acoustic washing.

Acoustic No Lysis Protocols

Immunophenotyping in blood is sometimes performed without red cell lysis by triggering detection on fluorescence signals rather than scatter signals. In these protocols, whole blood is stained with appropriate antibody and fed into a cytometer without lysis, in some cases with virtually no dilution. An acoustic cytometer according to one embodiment of the present disclosure is capable of performing this type of assaying with higher throughput of between approximately 100-500 μl of whole blood per minute since the blood cells can be concentrated into a central core with very little interstitial space. The white blood cells in normal patients usually make up less than 1% of the total number of cells in whole blood so coincidence of white cells in the dense blood core is rare. Hydrodynamic focusing cannot form such a solid core and can therefore not pass as many cells through a given cross sectional area. An additional advantage to formation of such a core is that all cells in the core travel at the same speed allowing for uniform transit times through a laser spot. The no lysis protocol can be further improved by adding an acoustic wash step that transfers the blood cells away from free antibody and into clean buffer. This reduces fluorescent background and increases sensitivity. In addition, the clean buffer can be adjusted to have an index of refraction that closely matches the cell's index. This has the effect of reducing scattering of the laser by the cell core.

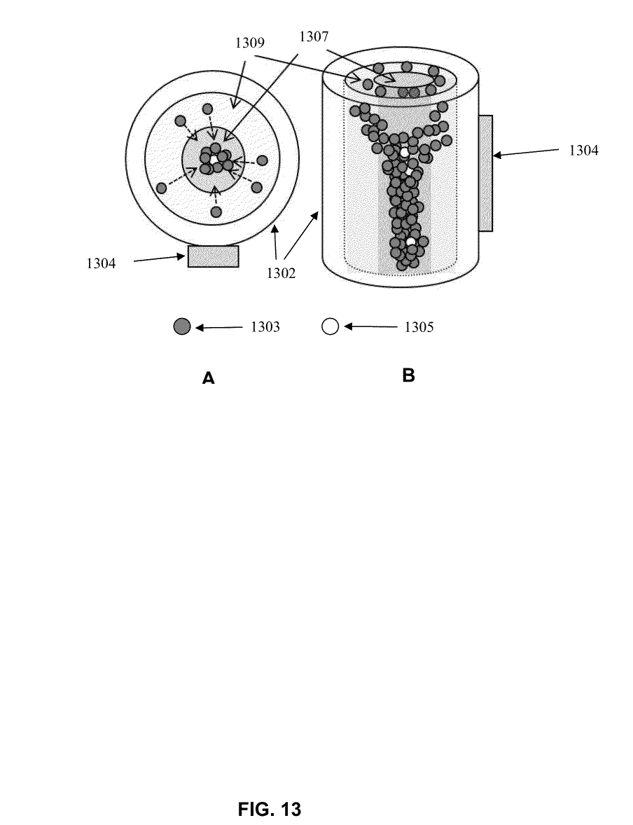

FIG. 13 is an illustration for stream switching of unlysed whole blood according to one embodiment of the present disclosure. Because of their relative low numbers white cells maintain separation in the rope like structure of focused blood. Capillary 1302 receives blood sample 1309 and wash buffer 1307 at different spatial locations of the capillary 1302. Red blood cell 1303 and white blood cell 1305 are acoustically focused and sample 1309 and wash buffer 1307 are acoustically reoriented upon activation of the transducer 1304 which produces an acoustic wave of a user defined mode within the capillary 1307. Cells are acoustically focused based upon their acoustic contrast.

### Example 4

### Urinalysis

Analysis of particles/cells in urine is a very common test used to screen and diagnose many conditions including urinary tract infections and urinary system cancers. Most commonly, particles/cells in urine are centrifuged to concentrate them and then they are examined using a microscope slide. This is time consuming, labor intensive and subject to operator error as well as error from the effects urine can have on cellular constituents.

Urine is a destructive environment for cells as it can have non-physiological osmotic pressures and pH as well as toxic metabolites. These conditions dictate a minimal post-collection delay for examination to avoid excessive degradation of cellular targets. This exposure can be minimized in an acoustic washing system by transferring the urine sample cells and particulates immediately into a cell friendly wash solution. The concentrating effect of the system is particularly well suited to urine processing where the cells and particulates tend to be of low concentration. Concentrated and washed fractions can be processed further as needed for a particular assaying. Reagents can be added, cells can be sent to culture or genetic analysis and/or an in-line analysis step can be added.

As urine density can vary widely, the wash fluid should be denser than the maximum density expected for the patient population tested (or compressibility should be adjusted accordingly). Urine sometimes contains mineral or other crystals that can be highly dense and a serial fraction that isolates these components with a very high density wash stream followed by a second, less dense wash to capture other components might be desirable in some cases.

### Example 5

### Coulter Volume Sensing/Electrical Measurements for Cells/Particles from Uncalibrated Solutions/Buffers

By acoustically transferring cells or particles into a solution that is calibrated for conductivity, particle counting can be done in line without centrifugation or dilution. This is of particular value for dilute samples where such manipulation by centrifuge may be difficult. It also enables automated continuous monitoring of some process. Monitoring particles in municipal water supplies is a good example as particulates are a very small volume fraction and continuous water monitoring might be desirable. One can envision a continuously operational system that might trigger more analysis as particles of certain size and characteristic increase. After triggering, for example, DNA dyes for revealing if the particles might be a biological threat can be added to the washing core and the particles would be sent to cytometric analysis.

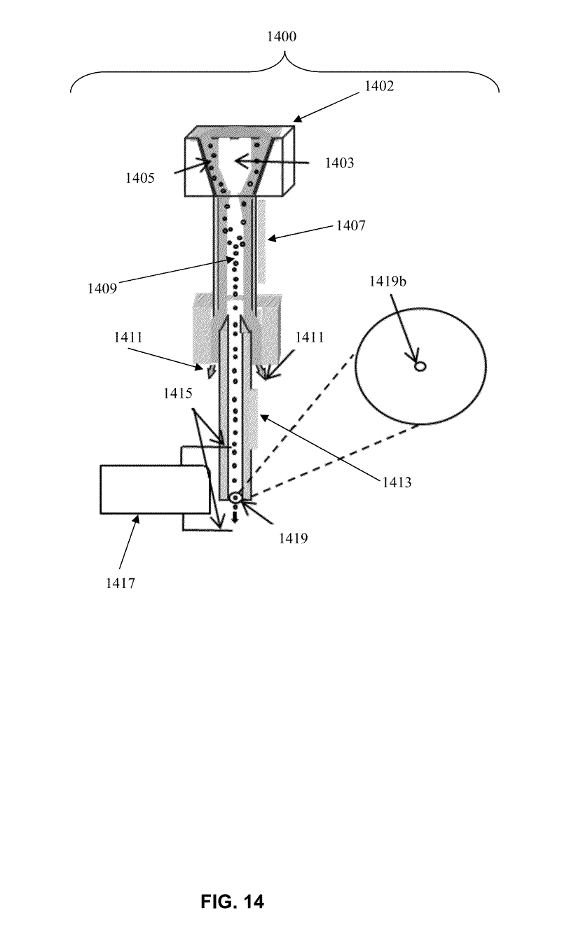

Acoustic focusing by itself provides further advantage to pore based electrical measurements as it ensures that particles to be analyzed pass through the measuring orifice in its center (FIG. 14). This makes measurements more precise and allows for smaller particles to be analyzed in larger less clog prone pores. This way, an instrument can cover a wider range of particle sizes without changing pore size as is the common practice.

FIG. 14 is a schematic example of an acoustic stream switching particle counting device 1400. The design allows for in-line analysis of samples 1405 in unknown or unusable conductivity buffer 1403. Even without stream switching the acoustic positioning of particles 1409 improves performance over a broader range of particle sizes for a given instrument pore size 1419. Transducer 1407 provides an acoustic wave to the flow cell. Particles 1409 are acoustically focused to buffer 1403. Sample medium is discarded at 1411. Electronics detection 1417 detects signals at electrodes 1415 after particles pass from the second transducer 1413 to the detection pore 1419.

### Example 6

### Bead Based Reactions, Purifications and Assays

Polymer beads, including but not limited to polystyrene beads, are very useful in embodiments of the present disclosure. Having a somewhat similar (slightly higher) positive acoustic contrast to cells, they can be manipulated in similar fashion. Being hardier than cells however, they can be subjected to harsher environments that might damage or disrupt cells. For example, high salt environments can be used in bead based immunoassaying to reduce non-specific antibody binding. This can be done with cells as well but salinity and or exposure time must be limited if membrane integrity is required.

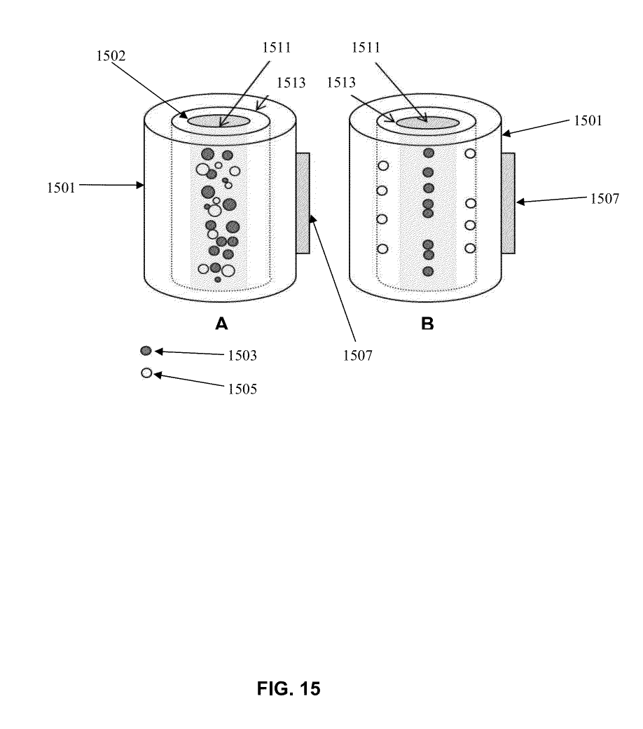

Beads of many different materials can be manipulated differently according to their acoustic properties. High specific gravity/low compressibility beads including but not limited to silica or ceramic beads can be acoustically focused through a high specific gravity central core that excludes the cellular debris in a lysis protocol. Negative acoustic contrast particles, e.g. silicon rubber, can be manipulated in opposite fashion such that they move to the outside wall of the capillary through a low specific gravity buffer, leaving cellular debris and uncaptured protein/nucleic acid behind in the center see (FIG. 15).

FIG. 15 is a schematic example of separation of negative contrast carrier particles 1505 from a core of blood sample 1503 and 1511. The negative contrast carrier particles 1505 leave the core 1502 and pass through clean buffer 1503 before approaching the capillary walls 1501. A transducer 1507 induces an acoustic wave that acoustically focuses the negative contrast carrier particle to the capillary walls and focuses the blood all to the center. Other acoustic modes exist that make it possible to invert the image. Blood cells can be driven to the walls and negative contrast carrier particles to the central axis

### Example 7

### Immunoassaying

Bead based sandwich immunoassaying benefit from acoustic wash in the same manner as immunostaining of cell surface markers. Centrifugation steps to eliminate excess antigen and reporter antibody are replaced with rapid in-line acoustic washing. The washed product is assayed in a conventional manner (e.g. bulk fluorescence, plate readers) or it is can be coupled to flow cytometry analysis, particularly if multiplexing using soluble bead arrays is desired. An apparatus useful to process samples for a plate reader provides all of the advantages of bead based assaying (inexpensive volume manufacture, better mixing and kinetics) with the existing infrastructure and easy calibration of plate reading assaying. Even enzyme linked assaying is carried out with the final amplification step being accelerated by active mixing with the beads.

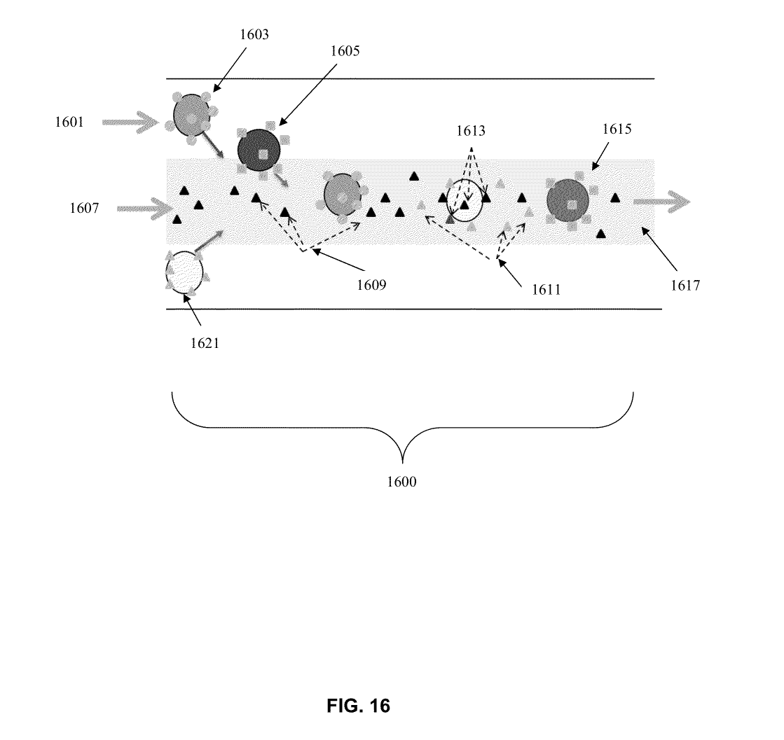

Competitive immunoassaying can be performed very quickly by flowing the analyte in the center stream and pushing beads pre-bound with fluorescent antigen into the stream. As the fluorescent antigen is displaced by native antigen from the analyte medium, the change in fluorescence of both the beads and the background stream can be monitored in real time. The beads become dimmer and the background becomes brighter as the fluorescent antigen is displaced and diffuses out into the stream (see FIG. 16). As flow rate in an acoustic cytometer is adjustable, the flow rate should be adjusted to match the kinetics of the assaying such that close to maximum signal is achieved at the detection point. By moving beads into the analyte stream and acoustically concentrating them therein, these problems of diluting the analyte, limiting assaying sensitivity, and long analysis time are eliminated. In addition, individual bead detection allows multiplexing for simultaneous detection of multiple analytes. Examples of such multi-analyte testing include but are not limited to blood donor screening, and/or STD testing.

FIG. 16 illustrates a schematic example of multi-plexed competitive immunoassaying in an acoustic wash system 1600.

### Example 8

### Staining of DNA/RNA Hybridized to Beads

Washing steps are eliminated for DNA/RNA prep and analysis as for protein analysis. Conventional labeling strategies using biotin or another linker are used with the final step being acoustic elimination of the reporter label prior to analysis. In addition, for native DNA or DNA without a linker, intercolating dyes are added to the wash stream in order to stain DNA hybridized to beads. Only double stranded DNA bound to beads are stained with this technique. This technique may be particularly useful for unamplified analysis of nucleic acid fragments (such as micro-RNA, plasmids or enzyme digested/mechanically fragmented genomic DNA).

These nucleic acids are extracted from a sample by hybridization combined with attachment of a high acoustic contrast label. The process includes hybridization of a probe with a linker including but not limited to biotin, followed by for example binding of streptavidin coated particles with high acoustic contrast including but not limited to silica, gold, or negative contrast silicone rubber. In this system, if multiplexing is desired, the probe itself may need to be coded in some readable fashion. Positive hits may only be recorded for signals that combined the coded fluorescence with signals from dyes intercolated into the hybridized nucleic acid.

Nucleic acids tests can be made more sensitive and specific if nucleic acid degrading enzymes are included in the transfer media (or a transfer medium in a previous step) and hybridized products are protected from degradation by protective modification of the DNA/RNA probes. In this way any nucleic acid not hybridized (including that non-specifically bound to beads) can be enzymatically degraded.

### Example 9

### New Cellular Analysis Tools/Applications

Acoustic manipulation of cells lends itself well to non-adherent cell line or cells that have been harvested from adherent culture. This combined with acoustic cell medium switching enables many new in-line manipulation techniques that enable new assaying, gentler and quicker handling of cells and lab automation.

Production of Fused Cell Lines

An important step in the production of fused cell lines such as tumor cell/dendritic cells or B-cell hybridomas for antibody production is getting the different cell types to contact each other prior to fusion. Line driven acoustic focusing in conjunction with optimization of sample concentration can be used to line up the different types of cells in separate lines of optimal spacing. These separate lines can then be joined by flowing them both into another focuser that then joins the two lines. Electric fields can then be used to fuse the cells either in flow or not. If desired this line can then be further analyzed or sorted prior to culture.

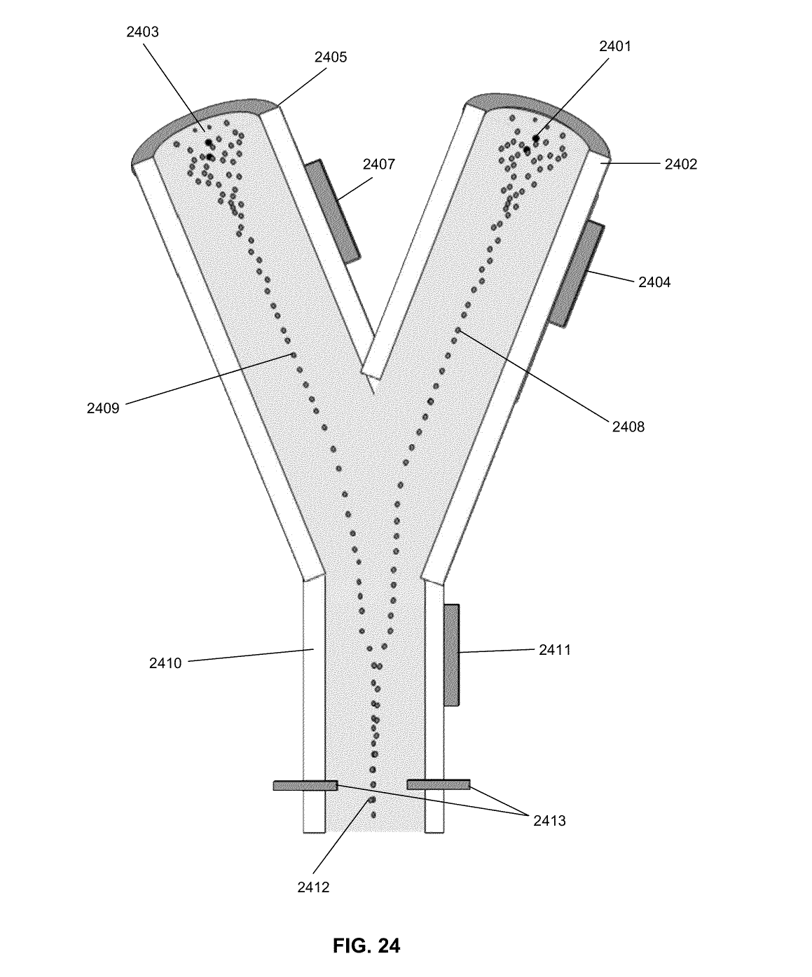

Referring now to FIG. 24, acoustic positioning of particles for fusion or reaction is illustrated. First sample 2401 containing a first cell or particle type is adjusted to an optimal concentration for interaction with a second particle type. The sample is pumped through first acoustic focuser 2402 driven by a PZT transducer 2404 and the particles are acoustically focused into a line 2408 with particles having a spacing according to the sample concentration. A second sample 2403 containing a second particle type is similarly adjusted for concentration and then pumped and focused into a line 2409 in the second acoustic focuser 2405 driven by PZT transducer 2407. The flow from both samples is flowed into a third acoustic focuser 2410 driven by PZT 2411 such that each focused line of particles flows parallel to the other. When the acoustic field from the acoustic focuser 2410 is switched on, the two separate lines of particles focus to form a single line where particles from sample 1 and sample two can interact. Acoustic Bjerknes forces in acoustic focuser 2410 act to bring close particles into contact. Downstream, after particles are in contact, the pass through an electric field produced using electrodes 2413. The electric field acts to fuse cells and the fused cells 2412 may be collected for culture or sent to another process such as analysis or triggered sorting. This device is useful for production of fused cells such as antibody producing hybridoma cells and can be applied to any process requiring interaction between suspended particles.

The method is not limited to fusing cell lines but can be applied wherever close interaction of particle populations is desired. Another important example is fusing aqueous drops in oil. In this case, the carrier medium is oil and the particles are the water droplets. Each drop population can be loaded with different reagents that interact upon fusion of the droplets. The droplets can also then be analyzed or sorted in flow. Other examples include exposing cells to beads with reactants as in T-cell activating antigens or exposing macrophage or monocytes to bacteria or other particles for phagocytocis.

Another embodiment of the present disclosure comprises joining three or more acoustically focused streams of particles in the same fashion by flowing them all into a single acoustic focuser where they are brought into close proximity be the acoustic field.

Still another embodiment of the present disclosure uses two acoustic focusers. The first focuser focuses the first particle population and feeds the line of particles into the center of a second acoustic focuser. The second population of particles is then fed around the axial edges of the second focuser and is acoustically focused into the center where they join the first line of particles.

Flow Through Affinity Harvesting of Cell Products

Affinity purification of cell products such as antibodies is typically accomplished using columns. Bead based methods using centrifugation or magnetic batch separation are also available. Acoustic separation can be used to accomplish this in a flow through fashion using affinity beads. For example, specific affinity beads such as those coated with an antigen of interest or protein A or G for capture of the Fc region of antibodies would be incubated and mixed with spent medium or anti-sera to capture antibodies. The beads can then be concentrated and collected in a flow separator and washed if desired. The wash medium may be formulated to discourage non-specific binding, e.g. high salt. The collected beads are then exposed to conditions which disrupt the specific binding after which they are again collected on a flow through separator where they can be recycled for the next purification. If desired the specific binding disruption can be accomplished in flow if minimal exposure to these conditions is desired. This is done by in-line acoustic medium exchange with the dissociating medium. The dissociated product is collected independently from the beads and processed as necessary, e.g. ammonium sulfate precipitation for antibodies or other proteins.

Harvesting of Cells or Particles

When the product to be harvested are the cells themselves, an acoustic separator can be used to concentrate and collect the particles with an axial collector or if the concentration of cells is high enough it can aggregate the cells into a continuous flowing line or line of clumps that can be fed into a collection vessel where flow is slow enough to allow settling by gravity or removal by other means. This method would be particularly useful for filterless continuous collection of microalgae for biodiesel production.

Harvesting of Lysis Products from Cells or Particles

If the cell or particle must be broken or lysed to harvest the material of interest, acoustic separators may be employed to separate lysis debris from the material and may also be used in-line to initiate lysis. Microalgae lysates are a special case where the product of interest, algael oil is separated and focused to the outside of the capillary and cellular debris and residual water goes to the center. If an appropriate lysis fluid is used, a simultaneous algae collection, lysis and algae oil step can be performed in which harvested algae are fed into one stream and lysis fluid fed into the other. Debris, lysis fluid and culture medium are collected in the center and oil is collected to the outside.

### Example 10

### Radio Ligands/Drug Candidates

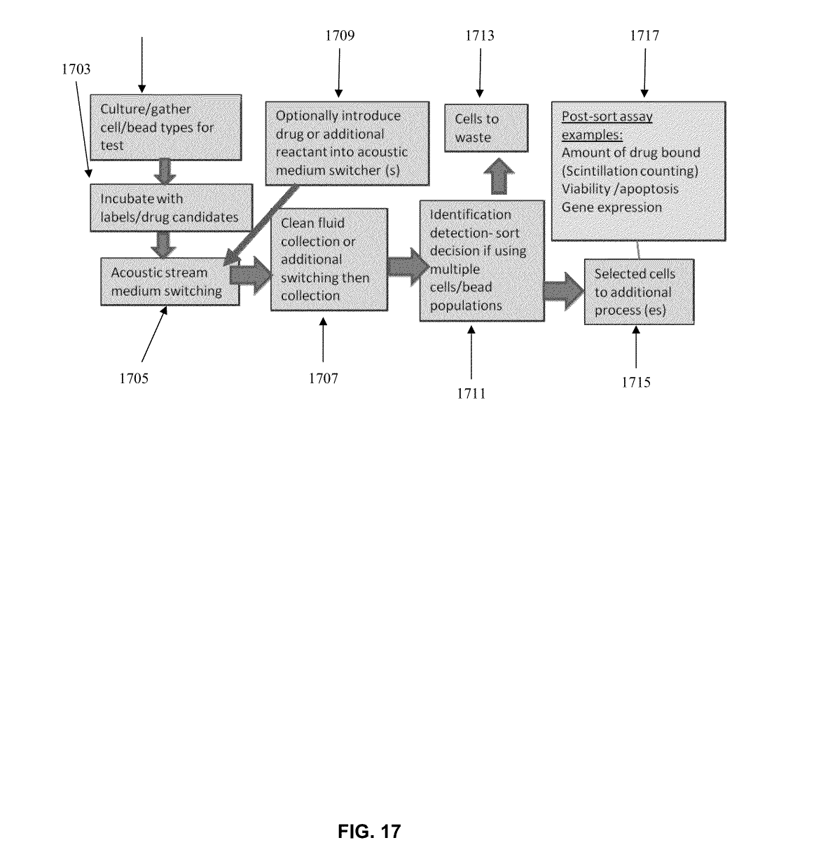

The ability to leave the original medium behind allows the combination analysis of long exposure time indicators such as radioactive ligands or drug candidates with single cell analysis and sorting techniques. For example, a radio labeled drug candidate can be added to a single well with several different cell types. Cell types incorporating positive and negative controls, including but not limited to cells from a parent line that have not been modified to contain the receptor of interest and cell lines with known activity. Relative cell size and granularity can be examined and multiple color analysis can be used to extract many parameters from each individual cell. Each cell type can be identified with cell specific fluorescent antibody combinations or with fluorescent fusion proteins/gene reporters, including stably expressing lines. A wide variety of intrinsically fluorescent reporter proteins and reporter protein systems that become stained with additional reagents may be used in the present disclosure. Many other reporters can be used to indicate cell conditions including but not limited to growth phase, pH, lipid related toxicity, etc. Receptor expression levels and internal fusion protein expression levels can be monitored, FRET interactions can be tracked. In short, any fluorescent parameter that can be monitored by flow cytometry can be utilized in the present disclosure. The multiplexed cell sample is washed in-line acoustically leaving excess radio ligands behind. The cells are then analyzed individually using acoustic flow cytometry and the analysis is sorted as to individual cell populations by fluorescence activated cell sorting (FACS). The collected population is then radioassayed for the amount of drug that remains with each population FIG. 17. If desired the cells can be acoustically transferred directly to a scintillation medium. With an acoustic flow cytometer, this process of washing, analyzing and sorting can be accomplished in fractions of a second, leaving little time for bound drug molecules to dissociate from cells. This process is particularly useful for drug candidates or other ligands that cannot be readily labeled with fluorescent or other large reporters without affecting activity.

FIG. 17 illustrates an example flow chart for high throughput/high content screening using acoustic medium switching. Cells or particles 1701 are incubated with labels and or drug candidates of interest 1703 after which they are sent to the acoustic focuser/stream switcher 1705 where they are separated from excess drug/ligand. Optionally a different reactant 1709 can be placed in the new medium such that cells/particles interact with it during acoustic separation. Additional acoustic switch steps can be added in serial as in FIG. 8. Cells are then collected 1707 for analysis and or sorting 1711. Unwanted cells/particles can be sent to waste 1713 while selected particles are sent to additional analysis or processes 1715. Some useful examples of such processes are listed in 1717.

Of course, the acoustic washing process can be used with most any reporter and is of particular utility for any application where the concentration of ligand should be maintained up until just prior to analysis. The process can also be extended to any ligand/drug candidate that can be assayed after analysis. If, for example, a library of proteins is synthesized with a sequence that allows fluorescent staining, the staining can be done after washing and sorting to determine how much was bound to the sorted population.

If only one cell population is used, sorting is not necessary, but data collected from the single cell analysis is useful in determining virtually any other parameter that can be monitored by fluorescent acoustic cytometry, e.g. number of live/dead or apoptotic cells.

### Example 11

### Calcium Activation

Acoustic washing of the present disclosure can be used to simultaneously simplify and improve calcium activation assaying or other ion probe assaying in flow cytometry. Calcium activation studies are normally done by preloading target cells with calcium sensitive reagents, washing away excess reagent and other media components that contribute to background fluorescence, exposing the cells to a calcium activator or drug compound under test and monitoring the cells for changes in optical signal. The assaying is often done quickly after the washing step in order to keep the concentration of the reagent within the cell high. “No wash” assaying has been developed to improve precision by maintaining equilibrium between intracellular and extracellular reagents but other reagents are used to reduce background such as probenecid which inhibits active transport of the reagent outside the cell or quencher dyes which reduce the fluorescence of extracellular dyes.

With acoustic washing, reagents and fluorescent media can be rapidly removed just prior to analysis, eliminating the need for quenchers or transport inhibitors. Washing need not be done prior to adding the calcium sensitive reagent. For example, cells may be maintained in a culture medium if desired and minimizes the use of other reagents that might interfere with the activator or the cell response is minimized. The calcium activator or test compound can be added to the acoustic wash solution or it can be added just prior to the acoustic wash depending on the users desired measuring time point. The reagents for use in an acoustically washed calcium activation assaying is then simply at least one calcium sensitive reagent and an acoustic wash buffer engineered to have an acoustic contrast higher than the cell sample medium. Examples of calcium probes are the Indo series, Bis-Fura, Fura series and FuraRed™, Bis Fura, MagFura series, BTC, Calcium Green™ Calcium Orange™, Calcium Crimson™, Calcium 3™, Rhod™ series and X-Rhod™ series, Magnesium Green™ and Oregon Green® BAPTA series. A second calcium indicator can be added to increase dynamic range measurements in a cell or a non-calcium dye can be added for reference. Combinations of dyes with reciprocal changes in fluorescence upon calcium activation have also been used to do ratiometric measurements with non-ratio-metric dyes, e.g. fluo-3 and Fura Red. Calcium indicators can be supplied in a number of different forms including cell membrane permeant AM esters and dextran conjugates designed to block internal cellular sequestration.

One embodiment of the present disclosure comprises a method for measuring cellular calcium concentration in an acoustic particle analyzer. This embodiment preferably introduces a calcium sensitive reagent into a population of cells to be analyzed. The population of cells is then moved through a channel wherein the population is acoustically focused in the channel. The population of cells is exposed to a reagent that may or may not induce a cellular calcium response. The population of cells is then preferably passed through an interrogation point and collecting signal to determine calcium concentration in the cell. In this embodiment, the population of cells can optionally be washed prior to analysis and/or diluted prior to analysis. The flow rate of this embodiment is preferably adjusted to achieve a desired time of analysis after the exposure to the reagent that may or may not induce a calcium response.

The power of flow cytometry to distinguish individual cells makes the prospect of engineering different cell types with the intent of simultaneously testing them for drug candidates or other activators very attractive. Each cell type can be engineered with different receptor types or receptor expression levels and can be uniquely identified using characteristic markers or other reporters. Different cell types can then be simultaneously mixed together and tested for response. Positive and negative controls can also be combined with various cell types being monitored.

The process can also be implemented in-line such that the ligand or an additional ligand(s) are serially injected into the core stream and the cells interact with the injected ligand. This is particularly useful for fast kinetic processes and can be combined with a kinetic analysis technique such as calcium sensitive fluorescence dye response. If radio labeled ligand is used, an additional in-line wash step might be required to eliminate the free ligand before sorting. Alternatively, a parallel system can be used where the cells pass through a layer of the ligand into the clean wash (see FIG. 12). In this system, interaction with the ligand will only occur as the cell passes through the ligand layer. Even if no radioligand is used, this medium switch method can be used to extract high information content as above in combination with calcium response or other kinetic analysis. The ability to adjust flow rates as desired enables tuning of each assaying to reach analysis at the desired time course. Kinetic response for a population of cells or beads can also be monitored by ramping flow rate up or down such that cells arrive at different times during the response curve.

A sensitivity problem for calcium response measurements lies in the ability to analyze quickly enough and for long enough after the calcium flux inducing ligand is added to catch and integrate the peak response of the cell population involved. The stimulant must be quickly mixed with the sample during analysis and analyzed cells end up with very different exposure times. With the acoustic media switching method of the present disclosure it is possible to precisely adjust flow rates such that cells arrive at analysis when desired and that they are monitored long enough to collect more signal. The method also insures that each cell in the population is exposed for the same length of time to the same concentration.

In addition to fluorescent parameters, any of these medium switch methods can also be combined with acoustic flow cytometric imaging which can provide additional valuable spatial fluorescence/luminescence information, morphology and spatially relevant absorbance information. The ability of the acoustic cytometer to drastically slow flow rates or even stop for a triggered event allows for high resolution imaging and high resolution spectroscopy, owing to the ability to integrate detection light for longer.

If cell population data is more important than individual cell data, many of the assaying protocols above can be performed in systems with simpler optics. Instead of probing single cells, a population can be monitored just after processing in a fluorescent/scintillation/luminescent plate reading device. Alternatively, reactions can be monitored inside the capillary by collecting light at either end.

### Example 12

### Low Affinity Drug Candidates/Ligands

Low affinity fluorescent ligands can be used in higher concentration as cells can be transferred from the high fluorescent background of excess ligand just before analysis in a time frame that does not allow significant dissociation. At high concentrations where non-specific binding of low affinity ligands may interfere with accurate analysis, acoustic washing into high salt buffers helps to favor specific reactions while accelerating non-specific dissociation.

### Example 13

### Multiple Ligands/Compounds and Serial/Parallel Processes

Much of biology and chemistry depends on the interaction of several different species of molecules or ligands. Acoustic medium switching provides a rapid and convenient means to expose cells or particles to a series of different compounds/ligands in rapid succession. This can be done in series and/or in parallel. It can also be readily modified to include changes in reactants or reactant concentration if the system is equipped to inject different media in the core streams or lamina of the devices.

Serial reactions can be performed for drug/ligand discovery in much the same way as shown in FIG. 11.

This can also be done in a triggered fashion in order to save resources. If for example a drug target is identified as a hit for inhibiting cell activity, it is then desirable to confirm the health and viability of the cells to exclude acute cytotoxicity of the compound. In this case, any “hits” can be diverted to a system performing viability testing. Alternatively of course, viability testing can be done simultaneously if a separately distinguishable health/viability marker is used.

### Example 14

### Enzyme Reactions

Enzymes are a special class of molecules that can be placed either in the original sample or in the medium that cells or particles are switched into. If cells or particles are acoustically transferred away from the enzyme, this will serve to stop an enzymatic reaction. If they are transferred into a medium containing enzyme, this will start the reaction. Enzymes are used for many protocols in sample prep, including but not limited to degradation of cell walls or unwanted nucleic acid. They are also used to detect or amplify detection of specific molecules including but not limited to fusion protein labeling or ELISA. They are also used as drug screening tools, e.g. candidates are monitored for their ability to block or inhibit the activity of specific enzymes. All of these applications are implemented in acoustic medium switching. In the last application for example, beads coated with fluorescent or FRET or quenched fluorescence enzyme substrate can be switched to a stream containing enzyme that was treated with a drug candidate. The fluorescence of the beads and the diffusing fluorescent substrate cleaved by the enzyme can be monitored much in the same way as described previously for the competitive immunoassaying (FIG. 16).

### Example 15

### Acoustic Medium Exchange for Bio/Chemical Synthesis, Bioreactors and Other Industrial Processes

Bio/Chemical Synthesis

Methods using cells or beads for sequential processes that require medium exchange can benefit greatly from the speed and automation possibilities of acoustically transferring particles across different media. Compounds synthesized on the surface of beads for example, can be transferred from medium to medium through the synthesis protocol. Another example is transfer of cells through media containing different growth factors designed to promote expression of a protein of interest in a sequential protocol.

Bioreactors

Ultrasonic concentration can be achieved, including but not limited to, antibody production using hybridomas and flocculation of microalgae for biodiesel production. The high-throughput, low-power capabilities of the round and elliptical radially concentrating systems described herein make these systems attractive for production scale bioreactor processing. The ability to perform media switching provide an additional benefit to many of these processes.

In antibody production, for example, spent antibody containing medium must be extracted from the valuable hybridoma cells, preferably without harm to the cells. Batch methods tend to produce higher concentrations of antibody but toxic metabolites generated during growth and centrifugation followed by resuspension of cells can be harmful to cells. This method also requires greater technician time and poses more contamination risks than continuous culture methods which use membranes or permeable capillary fibers for metabolite removal and nutrient replenishment

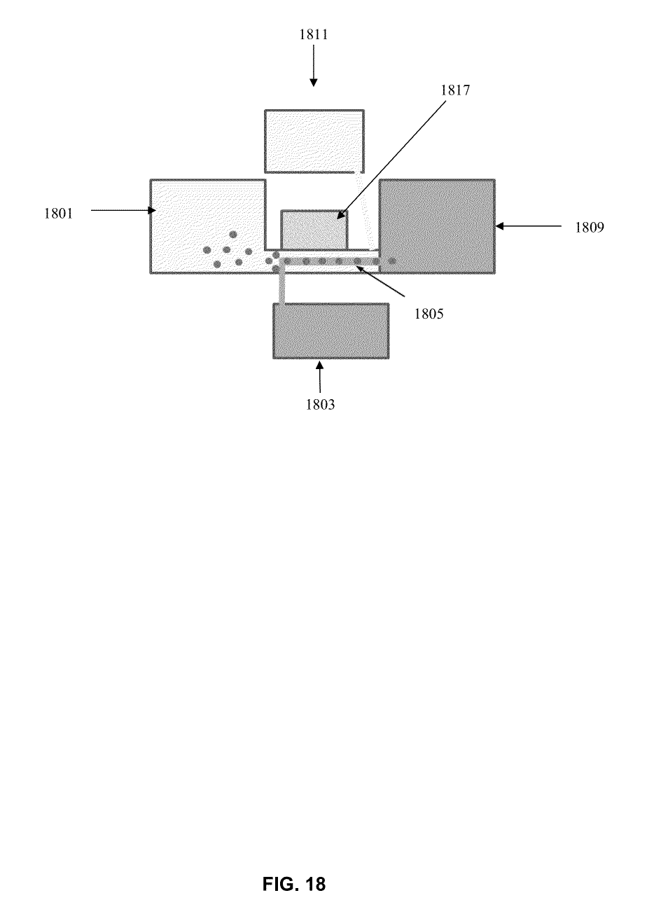

One embodiment of the present disclosure provides for acoustic medium switching to transfer cells directly into fresh medium at optimal cell growth times. If a second incubation chamber is used to begin a new culture, cultures can be continuously grown in a hybrid batch/continuous mode in which spent media is harvested while new media is seeded at optimal cell density. Excess cells can easily be removed from confluent cultures and dead cells can be removed acoustically as their acoustic contrast is lower. A simple light scatter detector can also be incorporated into the acoustic concentration device to monitor cell growth (see FIG. 18).

FIG. 18 illustrates a schematic example of a two chamber culturing/harvesting vessel using acoustic washing to harvest spent media and place cells in fresh media according to one embodiment of the present disclosure. The optical detector is used to non-invasively monitor cell growth at any time. Cells are cultured in chamber 1801 and periodically sent to be acoustically focused in the switching channel 1805. There they are examined for cell density/growth by the optical detector 1807. When growth and product production goals are met and the media is spent cells are sent through the channel 1805 and valves are activated to allow fresh media from the reservoir 1803 to be flowed along the axis of the channel and spent media to be harvested in a chamber 1811. The cells are focused into the fresh medium and transferred to the second culture chamber 1809 were the process can be repeated in reverse such that cells are cultured in the chamber 1809 and transferred into fresh media in the chamber 1801.

Any process requiring separation of viable cells to be recycled can be similarly implemented. Examples include but are not limited to cells, bacteria or yeast producing other secreted proteins, biofuel producing cultures such as ethanol and butanol producing yeast or bacteria. The acoustic separation process has been shown to be gentle on cultured cells and it enables automated continuously producing closed systems.

The high-throughput capabilities of round or oblate systems can also be put to good use in the harvesting of cells. For example, oil producing micro algae can be readily concentrated many fold at high flow rates with the process being readily scaled up by using multiple capillaries. The relatively large size of micro algae also permit high throughput for larger diameter, lower frequency capillaries.

### Example 16

### Bead Based Affinity Purifications

The utility of acoustic medium switching is not just limited to cell culture. Beads with selective coatings for the product of interest can also be envisioned in which for example the antibody is bound to protein A or G in an immunoreaction and is washed acoustically into a clean buffer where it can be removed and concentrated into the final product. This process can again be used for separation in further processes such as biotinylation or fluorescent conjugation.

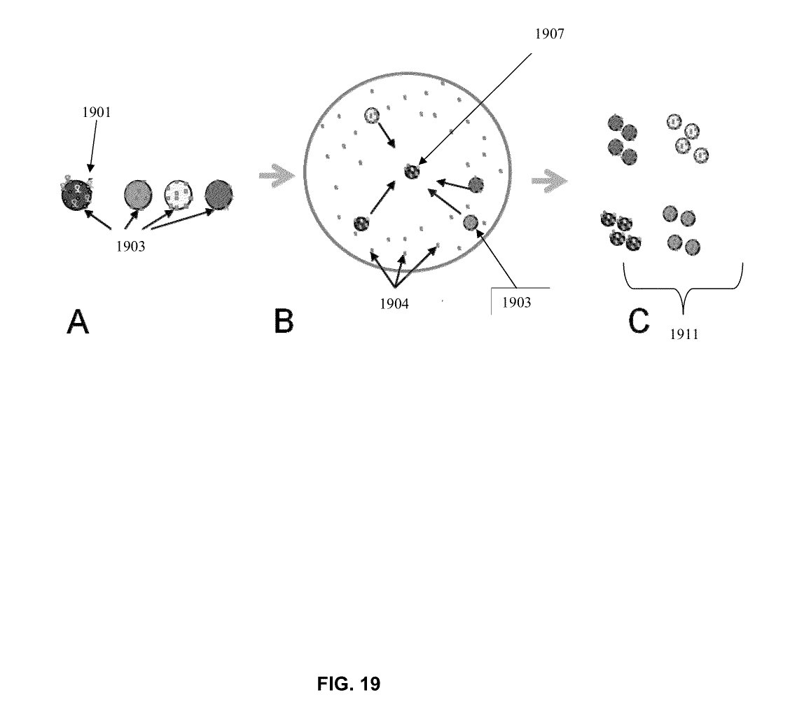

The system can be further extended for use in ligand library selection (e.g. aptamer or phage selection). The process also benefits from acoustically washing into a high salt or modified pH core where bead to ligand (e.g. aptamer or phage) affinity can be controlled to select the highest affinity ligands from a library. In this system the method of multiplexed fluorescent sorting can be applied to greatly increase the number of targets tested in a library, thereby saving time and utilizing expensive libraries to their fullest (see FIG. 19).

FIG. 19 illustrates a diagram of an aptamer selection from a library. FIG. 19A illustrates multiplexed beads or cells 1903 with target molecules 1905 incubated with aptamer library 1901. FIG. 19B illustrates in-line acoustic medium switching used to separate beads/cells 1903 from unbound aptamers 1904. Flow is into the page. Salt and or pH of the wash core (center circle) can be adjusted to select for higher affinity aptamers. Serial washes can be performed to increase purity. FIG. 19C illustrates beads 1903 and 1905 are sorted and the DNA/RNA 1901 bound to pure populations is amplified and process is repeated with the amplified aptamers. Subsequent rounds would focus on individual target molecules but other beads or cells might still be used to identify cross-reactivity of aptamers.

In general, beads with acoustic contrast to a medium can be used as an alternative to magnetic beads for virtually any purification process that magnetic beads are used. Beads used in acoustic separation can generally be made cheaper than magnetic beads. Larger magnetic beads also tend to clump together in a magnetic field and this can trap undesired materials. While few things in biological separations are magnetic and this gives magnetic beads a specificity advantage, the same can be said for negative acoustic contrast beads. Medium switching can also be accomplished for laminar flow systems with magnetic beads. There is utility in combining methods as well, particularly if more than binary separation is required. Three populations can be separated for example, if both negative and positive acoustic contrast particles were combined with magnetic particles.

### Example 17

### High Speed Valve Sorting

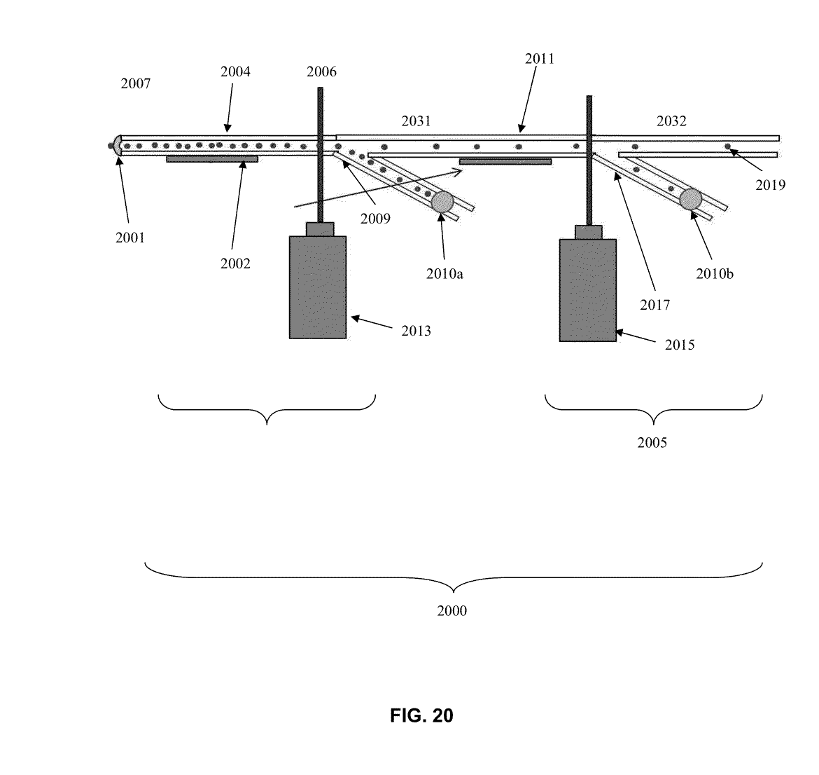

Acoustic medium switching of the present disclosure provides sorting of in-line washed cells and particles. While an acoustic medium switching module can easily be used with a conventional hydrodynamically focused flow cytometer, this new capability is made even more powerful by sorting methods that can be implemented in a sorting acoustic cytometer. Conventional cytometer sorting can be divided into two groups of instruments, droplet sorters and valve sorters. Most sorting tasks are performed by droplet sorters as they are generally much faster. Valve sorting cytometers do have advantages as they are gentler on delicate cell populations, they are less expensive and tend to operate more reliably without operator intervention. The shortcomings of conventional valve sorting cytometers include relatively slow sorting rates of 300-500 cells per second and dilution of sample with sheath fluid. Dilution with sheath fluid is also an issue for droplet sorters, but this is less problematic as the method allows capture of cells in very small droplets which can be diverted to tubes containing whatever medium is desired. The acoustic cytometer does not require sheath, so the dilutive can be eliminated. Also, since no sheath is required, sorting can be done in a sequential manner without further dilution of sample. For relatively rare cells, this enables a high speed initial valve sort that captures a cell of interest along with other cells for every sort decision. This enriches the ratio of desired cells and lowers the overall population in the sorted fraction. This sorted fraction can then be run again at a slower rate that will enable high purity of cells. If for example, cells are analyzed at a rate of 30,000 cells per second and the valve sort were capable of sorting at 300 cells per second, each initial sort decision should contain an average of about 100 cells. If these 100 cells are then transferred to a second sorter (or the same sorter after the initial sort) at a slower flow rate, the individual cell of interest can then be sorted with high purity. For an acoustic cytometer this repeated sorting can be done without additional sheath dilution and can even be done in an instrument that reanalyzes and resorts in-line (see FIG. 20). Dual or multistage sorting can be accomplished in-line because refocusing of cells or particles after the first sort can be accomplished using another acoustic focusing capillary. A conventional sorter would require a second sheath which would greatly increase fluidic complexity while continuing to dilute the sample.

FIG. 20 illustrates an example of a dual stage acoustic valve sorter 2000. This design enables in-line non-dilutive high speed sorting of rare cell populations. While similar “pre-sorting” strategies using repeated serial valve sorts are executed in conventional sorters, the hydrodynamic focusing of these instruments results in serial dilution. Sample 2001 comprising particle 2007 is introduced into part 2001 of system 2000. A first acoustic focusing system 2002 induces an acoustic wave in channel 2004. Interrogation source 2013, for example a light source interrogates the sample and/or particle at an interrogation point 2006. Particle of interest is sorted at 2009 and the unwanted particle is directed to waste 2012 paste valve 2010a. The kept particle or particles are directed or flowed in the stream to the second transducer 2002 where a second acoustic wave can be induced into the channel. The acoustically focused particle 2011 is interrogated at an interrogation point with a light source 2015 and optical information may be collected. A particle can be sorted to waste 2010b or sent for analysis or collection in the system 2019.

An alternative approach enabled by sheathless cytometry is the triggered capture of target cells. In this method a cell population can be analyzed at high speed and when a cell with the correct profile is identified, flow is stopped and the individual cell is collected.

In order for the many benefits of acoustic focusing to be fully realized, it is desirable for particle rates to be maximized for competitive throughput with conventional cytometers. This can be accomplished by adjusting sample concentration in the acoustic cytometer by prior dilution, in-line dilution or acoustic washing or a combination of these methods. For dilute samples, acoustic cytometry already has a distinct advantage, but this can be leveraged further by acoustic pre-concentration or washing prior to analysis.

Prior Dilution

The simplest method for reducing coincident rates in a large diameter acoustically focused channel for concentrated samples is to dilute the sample prior to processing such that the optimum concentration of particles is presented for analysis. Diluting the sample prior to processing gives the user tremendous flexibility in the initial concentration of samples that can be used with the instrument while insuring that the optimum rate of particle analysis for a given flow rate is achieved. Prior dilution can only be used in conventional cytometry for very concentrated samples before decreasing throughput. The particle analysis rates in an acoustic cytometer can be kept high not only because of the concentration effect but because systems have been developed that are capable of very high sample throughputs on the order of several milliliters a minute. It is the high volumetric sample rate possible in acoustic systems that makes this method fundamentally different from prior art methods.

### Example 18

For a 300 micron diameter acoustic focusing capillary, a 10 microsecond transit time through the interrogation laser, and a particle rate of 10,000 particles/s, a concentration of about 2.8×105 cells/ml or less is required to achieve a mean event rate of less than one in ten time windows. According to Poisson statistics, this corresponds to a probability of about 1% that a time window will contain more than one event meaning about 10% of events will be coincident. This sample is less than half the concentration than the example above for a conventional cytometer where particle rate was limited to 100 particles per second. The volumetric flow rate required for this 10,000 particle/s rate example is about 2.1 ml per minute. An acoustic cytometer can maintain similar precision of focus to the slow sample rate of a conventional cytometer for cell sized particles at this greater volumetric flow rate.

For an acoustic cytometer with a 300 micron diameter, a concentration of about 2.8×10⁵ cells/ml is optimal for maximum throughput with about 10% coincident events. For larger particles or larger laser beams or if fewer coincident events are desired, a user might choose to reduce coincident events by decreasing concentration. Samples run on an acoustic cytometer with a flow rate of 2.1 ml/min can be diluted up to 210 fold before more time is needed to process the sample than for a conventional cytometer running with a sample rate of 10 μl/min. Thus, with simple up-front dilutions, an acoustic cytometer can operate at higher throughput than a conventional cytometer for concentrations up to about 6×10⁷ cells/ml. For higher concentrations, throughput cannot be increased beyond the maximum particle rate of a given instrument.

The 6×10⁶ cells per ml concentration sample can be conventionally processed at a maximum rate of 1000 cells/s. An input rate of approximately 10 μl/min is typically diluted about 20 fold to reach the optimum concentration for an acoustic cytometer. By running at 2 ml/min, particles are analyzed at nearly 10 times the rate of a conventional cytometer. If a user prefers to take advantage of longer transit times through the laser, a sample could be slowed to 0.2 ml per minute where it would have similar particle analysis rates to the conventional cytometer but with much longer transit times.

Prior dilution of samples with concentrations greater than an optimal concentration for a given acoustic cytometer allows the use of nearly any concentration of starting sample and it allows pre-treatment of buffers in any number of ways including adding reagents or changing acoustic contrast, dissolved gas content or temperature. It also conveys other valuable assaying benefits. Among these are background reduction from unbound labeled ligands and decreasing the minimum size sample required.

Background Reduction

When staining cells or particles with fluorescent antibodies or other ligands, it is desirable to bind as much of the ligand to the target as possible while leaving as little as possible in solution. The remaining labeled ligand causes fluorescent background that reduces the signal to noise ratio during analysis.

It is usually advantageous to do the staining in a small concentrated volume to favor binding kinetics. Simply diluting the sample reduces the concentration of unbound antibody and therefore the background signal during analysis. Dilution is not generally performed in conventional cytometry because it decreases particle rate thereby increasing analysis time. For many samples in cytometry, the cell concentration is already greater than a typical optimum concentration for an acoustic cytometer.

After staining, centrifugation followed by resuspension in a new buffer or medium is often performed to eliminate unbound ligands. This can still be done for an acoustic cytometer but it can also be coupled to dilution by simply adding more buffer. This process makes the unbound ligand concentration even less than with centrifugation alone.

Sample Size

Conventional cytometers usually require volumes on the order of a few hundred microliters to function properly and often some of the sample cannot be analyzed when the level runs low. Even newer cytometers with smaller sampling capabilities boast a minimum sample volume of 10-25 μl. Using dilution, nearly any starting volume is possible in an acoustic cytometer of the present disclosure. For example, the 210 fold dilution factor mentioned above makes a 1 μl sample into a 210 μl sample which can easily be processed with an acoustic cytometer using standard sample tubes.

One embodiment of the present disclosure comprises very small samples in microtiter plates. Well plates typically only have a maximum volume up to about 20 μl so dilution of a 1 μl sample can only be done up to 20 fold. If however, diluent is fed to the well while the sample is being fed to the cytometer, a higher fold dilution can be accomplished.

Kinetic Dilution

The rapid volumetric processing rate of an acoustic cytometer of the present disclosure also allows dynamic experiments on time scales that conventional cytometers cannot achieve. If a diluent containing a reagent(s) or drug candidate is mixed with a sample just prior to analysis, processes triggered by this reaction can be monitored for the entire sample over a very short time period. If for example a 10 μl sample of cells with a starting concentration of 2×10⁶ cells per ml is primed with probes for monitoring calcium activation, and 90 μl of diluent containing a calcium activator or test compound is added just prior to analysis, the entire sample can be analyzed in an acoustic cytometer in approximately 6 to 30 seconds at a flow rate of 1 to 0.2 ml/min. For the same sample in a conventional cytometer, it would take at least 60 seconds to analyze with no dilution at the cytometer's top, less precise sample rate.

One advantage of the large dilution that is allowed in acoustic cytometry of the present disclosure is that rapid mixing can easily be accomplished. This ensures that all cells have equal exposure to the reagent and the cell reaction over time can be more accurately monitored.

Combining Pre-Dilution with In-Line Dilution

While prior dilution has many advantages, it is also sometimes desirable to combine predilution in the present disclosure. One example is when a large volume of concentrated sample must be processed from a small sample tube. A 1 ml sample with 2×10⁷ cells per ml needs to be diluted in a conventional cytometer 100 fold to achieve a 10% coincidence rate and this requires a 100 ml sample volume. With an acoustic cytometer, it is possible to drastically change the in-line dilution from nothing to ratios of diluents to sample similar to conventional hydrodynamic focusing without degrading the precise focus.

A variable diluent can be employed with pre dilution such that virtually any combination of acceptable coincidence is achieved. For the example above, if a 10 μl sample tube were available, 10 fold predilution could be combined with 10 fold in-line dilution, or 5 fold pre-dilution might be employed with 20 fold in-line dilution to accomplish the same thing.

The sample inputs can be configured in a number of ways for in-line dilution. If the sample flows in the center of the flow cell for acoustic focusing, and the diluents surround it coaxially, some of the benefit of unbound probe dilution will be lost but the in-line diluents will keep the sample from contacting the walls and will also force particles into starting positions in the flowed where the acoustic gradient is higher. This allows greater throughput and better focusing of smaller particles.

In-line acoustic washing of particles can also be employed to the same effect if the sample fluid is of lesser acoustic contrast than the diluent's fluid. In this case, depending on the initial flow configuration, the sample fluid itself moves toward or is maintained at the walls of the focuser, while the particles or cells are retained at or are moved to the central focus.

Combining Analysis with Off-Line Acoustic Washing/Concentration

In another embodiment of the present disclosure, analysis can be done after acoustic washing and/or acoustic concentration is performed offline. In an acoustic washer/concentrator cells or particles can be concentrated many fold while discarding most (concentration) or nearly all (washing) of the original medium. This is of course of particular utility when the original concentration is sub-optimal for the desired particle analysis rate, but it is also of great utility for samples with very high background or for samples that require a high degree of background reduction.

Once a sample is washed or concentrated, it can then be diluted or not depending on concentration and desired particle coincidence vs. analysis rate. It is often difficult or impractical to keep careful track of the precise concentration of a sample so a user may employ an aid such as a spectrometer to determine concentration based on light scatter. Alternatively, another embodiment of the present disclosure includes an on-board spectrometer that can calculate the proper dilution and possibly also execute the dilution automatically. Still another embodiment of the present disclosure, allows a user to take a portion of the sample or a diluted portion and run it on the instrument to determine concentration and dilution prior to the main analysis.

Transit Time Advantage

For a conventional cytometer, transit times through an interrogation laser are usually about 1-6 microseconds. With an average event rate of 0.1 per unit time, 10 microseconds corresponds to an analysis rate of 10,000 particles per second. For acceptable coincidence and an event rate of 1000 particles per second, an acoustic system of the present disclosure can accommodate transit times of 100 microseconds, a range that greatly improves photon statistics and opens the field of application for the longer acting photo-probes.

Assaying for cells, particles and microbes can be improved using acoustic focusing with the pre-dilution method, in-line dilution method or in-line or offline acoustic concentration or washing method or combinations thereof. Both assaying with higher sensitivity/resolution and novel assaying made practical by acoustic cytometry greatly expand the capability of analysis in flow.

General examples of assaying that can use acoustic cytometry according to embodiments of the present disclosure include, but are not limited to cell sorting, apoptosis analysis, cell cycle studies, fluorescent protein detection, cell proliferation assaying, immunophenotyping, antigen or ligand density measurement, gene expression or transfection assaying, viability and cytotoxicity assaying, DNA/RNA content analysis, multi-plex bead analysis, stem cell analysis, nuclear staining detection, enzyme activity assaying, drug uptake and efflux assaying, chromosome analysis, membrane potential analysis, metabolic studies and reticulocyte and platelet analysis among others. Assaying can be improved using acoustic focusing fluid reorientation or a combination thereof using an acoustic cytometer with the additional steps of adjusting to the desired optimal throughput concentration through prior dilution, in-line dilution and or acoustic washing and selecting the appropriate transit time for best results. In addition, an acoustic cytometer that has slow or stopped flow imaging capabilities provides additional flexibility and advantage. Additionally, off-line concentration can improve throughput where cell concentrations are sub optimal. Off-line acoustic washing can also replace most centrifugation steps or can be added as a background reducing step.

Non-Compensation Protocols

An embodiment of the present disclosure comprises a method for reducing compensation in an acoustic cytometer. This embodiment includes flowing particles with at least 2 fluorescent labels through the acoustic cytometer and collecting fluorescent signals from the particles as they pass an interrogation point. Then overlap from different color fluorescent labels is reduced by using at least one fluorescent band filter with a narrowed band pass such that signal from at least one fluorescent label emission is reduced. The transit time is then slowed by reducing the flow rate such that at least as many photons are collected from the reduced signal as when the wider band pass filter is used with a faster transit time. Assaying of this embodiment preferably uses at least 2 fluorescent labels and can run without running compensation controls and without compromising results.

Analysis of Microbes

Analysis of very small particles and cells is a considerable challenge for conventional cytometry. Quantities of proteins and DNA are on the order of 3 orders of magnitude smaller then for organisms such as bacteria. The longer transit times in acoustic cytometry improve microbe analyses by improving the photon statistics of these dim measurements. This improvement provides for many new methods of microbe analysis in an acoustic cytometer that could not commonly be measured in conventional cytometers.

Reductions in instrument cost that are made possible by acoustic cytometers also make routine counting and live/dead type analyses of microbes much more accessible to more researchers.

Low cost analysis for mammalian cells may be done with the present disclosure, beyond counting and viability such as apoptosis and cell proliferation.

Methods for Increasing Dynamic Range

Increasing dynamic range can be important for assaying in which there is a wide range of signal intensity, there are increasing numbers of distinguishable populations in a bead set and using detectors that have a more limited dynamic range. Photo-multiplier tubes are dominant in cytometry. They have a wide dynamic range but lower quantum efficiency than some lower dynamic range detectors including but not limited to avalanche photodiodes (APDs). Multi-pixel APD devices known as silicon PMTs may also be used in the present disclosure.

With the long transit times in an acoustic cytometer, greater dynamic range is available than in fast transit time systems because of the added dimension of time. Two lasers of different power and different spatial location may be used to analyze the same particle twice. The stronger laser is used to quantify the dim particles while the weaker laser is used for stronger particles. For a longer transit system, a similar increase in dynamic range can be realized by using a single weaker laser, increasing transit time and measuring pulse area (integrated signal from photons). Alternatively, instead of, or in addition to, measuring peak height, the rate of signal increase or decrease of the pulse can be measured. For a large signal, if this information is taken prior to detector saturation, the expected brightness can be calculated rather than measured. This method is particularly useful for beadsets with set ratios of coding labels as the ratio(s) of rate increase can be used to decode the population without regard to intensity. Also, an extremely wide range of staining concentrations can be used without saturation of the detector.

The longer transit times also make increased dynamic range from a single modulated or pulsed system more practical. In a modulated system, dim particles are measured at peaks and bright particles are measured at valleys. To use a single laser for a pulsed system both stronger and weaker pulses need to be administered at different times. For a pulse rest method, this is practical for long transit times but not short transit times where the rest period is a significant fraction of the transit time. In a pulsed system, peak intensities can be very high and can damage certain types of photodetectors, so care must be taken in photodetector selection.

Still another method for increasing dynamic range is decreasing the color bandwidth of filters. The most common example of this is a linear detector array in a spectrometer used in conjunction with a dispersive element including but not limited to a grating or prism. While this limits the number of photons per detector and therefore decreases precision due to photoelectron statistics, it allows brighter signals without saturation and can also be used to reduce compensation requirements in multi-color assaying and reduce signal to noise by collecting a higher ratio of signal light to background light. If for example one constructed assaying using AlexaFluor® 405 and Qdots®: 545, 585, 655 and 800, there would be some spectral overlap of fluorophores but uncompensated detection can be used if some narrow band pass filters were to be used.

Multi-Parameter Detection

Acoustic cytometry according to systems and methods of the present disclosure not only adds to dynamic range but it can add dimensions to assaying multiplexing by allowing enough time for other optical phenomena to be monitored, including but not limited to luminescence and/or chemi/bio/electrical luminescence. With the previous example, if a metal ligand complex including but not limited to europium chelate is a sixth label and a pulsed light source, the first five colors can be monitored just after the pulse and the Europium can be measured throughout its decay lifetime of several hundred microseconds. The narrow primary emission of the europium at 613 nm overlaps some with the emission spectra of the 585 and 655 Qdots® but it would not be detected in these channels if a narrow emission bandpass filter is applied to the Qdot channels. With this combination, six colors are possible with virtually no compensation. In general, long emission fluors can be automatically compensated for even if there is bleed over because relative contributions can be determined on the basis of time and can be subtracted appropriately. Given the complexity of controls and computing power required in typical six color flow assaying, an embodiment of the present disclosure provides for compensation free or minimal compensation reagent kits, even down to two colors.

Assaying is often processed in a single sample, multi-parameter detection can have great utility. Short lived fluorescence intensity beads can be used as an assaying identifier and long lived fluorescence lifetime as a reporter. With longer transit times and optimized throughput, there are many useful applications. If, for example several shorter lived probes are incorporated into a beadset with varying intensities, the number of possible combinations is such that the beadset can compete with conventional high density nucleic acid arrays. With luminescent reporters, very high sensitivity is possible even with highly fluorescent beads. Additionally, the combination of Qdots® and metal ligand complexes can be efficiently excited with a single violet source including but not limited to a 375 nm laser diode.

Auto Fluorescence Correction

Auto-fluorescence is often a problem for sensitive detection of small numbers of labels. It has fairly broad emission and can spill over into many channels. In multi-colored applications, it adds another parameter that must be compensated outside of the multiple labels to be used. Just as longer transit times can help improve coefficients of variation for labels with better photoelectron statistics it can also help reduce variance from background such as auto-fluorescence. The net result is that signal to noise ratio is improved as the variance of both signal and background is narrowed.

Auto-fluorescence subtraction has been demonstrated using two lasers. The first laser excites auto-fluorescence above the wavelength of the excitation laser, and the signal detected above that wavelength is used to estimate the auto-fluorescence contribution expected for the primary detection laser. Auto-fluorescence can be done with a system having a violet laser and a blue laser. It can also be done with a system that has only a violet laser or is using a violet laser to excite more than one color, if there is a separate color band to monitor the auto-fluorescence. Only the blue fluorescence channel is monitored, and expected contribution in other channels is subtracted. For pulsed or modulated systems with long lifetime probes, the short lived contribution of the auto-fluorescence combined with the initial output of the long lifetime probe is measured. Fluorescence of the long lifetime probe after the auto-fluorescence has decayed is also measured and back calculated to determine the auto-fluorescence contribution in all channels.

### Example 19

Four color assaying with only auto-fluorescence compensation can be performed using four different Qdots®: 525, 585, 655 and 800 and a single violet diode laser. These Qdots® have very little spectral overlap and can be easily separated. If a second laser, including but not limited to an inexpensive diode such as 650 nm or 780 nm is added, other combinations that are virtually compensation free can be added with even more colors. For example, Qdots® 525, 565, 605, 705 and AlexaFluor750 which is excited very efficiently at 780 nm can be added. The 800 Qdot® is not chosen in this case as it has some excitation at 780 nm. For this five color combination, narrow band filters are used to prevent overlap between Qdots®. If elimination of compensation is not critical, similar strategies for employing low cost diodes can be used effectively with more conventional dye combinations such as pacific blue AlexaFluor405®/Cascade Blue® and pacific Orange® off the violet diode and APC and APC AlexaFluo®700 off a 650 nm diode. Alternatively, 473 nm DPSS blue lasers are reasonably inexpensive when they have RMS noise levels of a few percent or more. The long transit times afforded by an acoustic cytometer enable noise integration that can make these lasers attractive. These lasers can then be used in place of the most common 488 nm wavelength lasers where they are capable of exciting the most common fluorophores. Green DPSS modules e.g. (532 m) are even less expensive and less noisy and can be used to excite PE and its conjugates more effectively than even the 488 nm wavelength. In a system where emissions off of each laser are kept distinct, either by spatial or temporal separation, one can use several colors from each laser. If the pulse/rest method is used, lasers can be co-located and fired in sequence. Fluorophores that have little absorption in bands that are being pulsed are still able to rest. If, for example, the rest period is one microsecond and four different lasers are used with 10 ns pulses, each laser is triggered every microsecond with a pulse of a different wavelength hitting the target every 250 ns. A second low power pulse for each laser can be used to extend dynamic range (brightest signals are quantified from the low power pulse, dimmest from the high power pulse). Using lasers at 405 nm, 532 nm, 650 nm and 780 nm four colors and autofluorescence can be monitored with virtually no compensation: 405 nm-autofluorescence and Pacific Orange, 532 nm-PE or Cy®3, 635 nm-AlexaFluor®647 and 780 nm-AlexaFluor®790. There is some excitation of PE at 405 nm and some excitation of AlexaFluor®790 at 635 nm so slight compensation might be required. If compensation need not be eliminated, several colors can be excited off of each laser. With lasers collocated but separated temporally, one can use the same detectors where dye emissions from fluorophores excited by different lasers overlap.

Many of the 405 nm violet laser diodes are typically high quality with low noise. Since these diodes can be obtained inexpensively with high pulsed powers, they useful for implementing high power pulses with long rest times. The diode wavelength of the 405 nm violet laser can be very useful with or without pulses when coupled with long transit times. It is very efficient for excitation of quantum dots which is useful for many-colored assaying. This, coupled with narrow band emission filters, is useful for assaying with little or no need to compensate.

### Example 20

### Method for Luciferase Mediated Gene Detection in an Acoustic Cytometer

Another embodiment of the present disclosure provides form bio or chemiluminescence in an acoustic cytometer and the detection of gene expression, for example, using luciferase as a gene reporter. While gene expression detection can be accomplished with other means such as fluorescent protein expression, bio/chemi luminescence adds an additional parameter that can be separated in time from this or other flow cytometry fluorescence parameters. Light generated by the reaction of gene expressed luciferase and its substrates luciferin (or coelenterazine for Renilla luciferase) does not require external excitation and is therefore free of autofluorescence excited in the cell, flow cell or detection optics. This also makes luciferase especially useful for detection of low level gene expression where signal to noise is especially important. In general, cells expressing luciferase are loaded with luciferin which is generally cell impermeant except for specialized reagents such as caged DMNPE luciferin which can be loaded into the cell by incubation. They are then supplied ATP which completes the light producing reaction. For DMNPE luciferin can be uncaged using UV light.

Using an acoustic cytometer with a pulsed excitation system, it is possible to sensitively monitor the chemi-luminescence between laser pulses. Standard fluorescence flow cytometry parameters can be collected as desired to determine cell characteristics including cell surface markers, detection of fluorescent protein gene expression, calcium activation, nucleic acid analysis and so forth.

Luciferase Antibodies and Secondary Reagents

Luciferase can also be conjugated to antibodies and secondary reagents like protein A and G. Avidin and streptavidin recombinant protein A and streptavidin luciferase fusion proteins have also been developed. These reagents can be used to label cellular antigens or bead bound targets in order to add an additional parameter for analysis in acoustic cytometers. With an acoustic wash containing luciferin and ATP, systems with pulsed lasers can detect the luminescence between pulses and subtract this quantity of light from overlapping spectra of fluorophores used to measure other targets. This is especially useful for multiplex beads sets that rely on fluorescence for coding. It is also especially useful for measuring low levels of antigens on cells with high autofluorescent background. In addition, luciferase can be used in conjunction with any laser combination or even in the absence of lasers as it does not require excitation light.

Materials for Engineered Acoustic Media

According to another embodiment, methods of acoustic washing particles and reorienting fluid utilize media formulations that have higher acoustic contrast than the sample medium. For many biological samples the medium is buffered saline, often with protein, detergents or other additives. Many media with higher acoustic contrast than physiological saline have been developed for use in density gradient separations by centrifugation. The functional constituents of these media are salts and proteins combined with additives used to increase specific gravity without undue increase in salinity. Commonly used examples include sucrose, polysucrose, polydextran, glycerol, iodinated compounds like amidotrizoate, diatrizoate, iohexyl (Nycodenz®), iodixanol, ioxaglate, iopamidol, metrizoate, metrizamide, and nanoparticulate material such as polymer coated silica (Percoll® for example). In applications where high salinity is not a problem, the primary constituent is a heavy salt such as cesium chloride or potassium bromide.

For acoustic separations density and osmolarity are important but additional parameters such as compressibility and viscosity are more important than for centrifugation media. This makes the priorities for formulation of acoustic separation media different. Viscosity is of higher concern than for centrifugation as higher viscosity dissipates more acoustic energy relative to lower viscosity. Therefore, compounds that contribute to high viscosity are not preferred unless required by the application. In general sucrose/polysucrose, glycerol and dextran fit into this category. Nano silica coated with polyvinylpyrrolidone is also highly viscous and fluorescent as well. Preferable compounds to be added include the iodinated compounds above and are preferably selected not only on the basis of contribution to viscosity and osmolarity but also on compressibility. Metrizamide, Nycodenz®, diatrizoate and iodixanol are useful for altering the acoustic contrast of a fluid.

In preparations where physiological osmolarity is desired but short term loss of physiological ions will not be important, a heavy salt, such as cesium chloride but not limited thereto, can be substituted or partially substituted for other salts such as sodium chloride. Cesium chloride provides a benefit not only because it is an innocuous and relatively heavy but because it reduces viscosity of the medium. A cesium chloride solution with physiological osmolarity has about 3% lower viscosity than a comparable sodium chloride solution. This is an advantage for acoustic separations according to one embodiment where higher viscosity absorbs more acoustic energy.

Cesium chloride is useful for acoustic separations that can tolerate high salt such as separations of fixed cells and beads. High salt acoustic wash buffer combined with additives such as protein and surfactant or detergent can be used to minimize nonspecific binding in both protein and nucleic acid assaying. One preferred embodiment uses cesium chloride and a Pluronic® non-ionic surfactant such as Pluronic®F68. The Pluronic® has very low auto-fluoresence and is therefore well suited to flow analysis.

Beads with Lifetime Auto-Calibration

A difficult problem for assaying in flow cytometry is absolute quantification of analytes from instrument to instrument and day to day or even minute to minute. Differences in laser power and fluctuation, PMT adjustments and degradation and flow alignment are among the worst culprits in variability. Absolute quantification must typically be done using calibration beads that excite and emit in the same channels as the analyte to be detected. An alternative to this procedure can be accomplished in an acoustic cytometer with lifetime discrimination capability using beads loaded with a known amount of long lifetime fluorescent dye (preferably greater than 1 microsecond). The long lifetime dye is excited simultaneously with the analyte probe and after short-lived fluorescence dies down (typically 1-100 μs), the remaining signal of the long lifetime probe can be used to calculate the signal of the analyte probe. The initial signal of the long lifetime probe is calculated from the known lifetime curve of the dye and is subtracted from the combined fluorescence peak of the analyte probe and the long lifetime dye. Alternatively, if the analyte is probed with a long lifetime dye the bead reference dye can be short lived. Absolute calibration is easiest when the analyte and reference dye are excited by the same laser and detected by the same detector so the probes need to be selected with this in mind. The commonly used lanthanide chelates are generally UV excited so their utility is limited in systems with visible lasers. Other suitable candidates include but are not limited to metal ligand complexes using metal ions such as europium, terbium, samarium, iridium, ruthenium, neodymium, ytterbium, erbium, dysprosium, platinum, palladium, and gadolinium. The excitation, emission and lifetime properties of metal ligand complexes are dictated by the metal ion and its ligand. Coupling different ligands to different ions and/or modifying ligand structure has been and continues to be heavily researched. A wide variety of possibilities are available for tuning of lifetime and excitation and emission wavelengths.

In one embodiment of the present disclosure, reference beads can be formulated for any of the systems described previously by combining compatible optical parameters. For example absorptive dyes can be used for coding while a short-lived fluorescent dye is either used for reference or detection and a long-lived dye is used for reference or detection. A UV excitable short lifetime dye including but not limited to Pacific Orange®, or a quantum dot and long-lifetime probes including but not limited to terbium chelates or europium chelates or tandems thereof, are good choices for single source excitation of the reference and detection components. Absorptive dyes can be selected from a wide list of non-fluorescent species but they preferably absorb in spectral regions away from the reference and detection probes excitation and emission such that even very heavily dye loaded beads do not absorb the excitation or emission light. Good choices would absorb in the infrared or near infra-red region. Low cost diode lasers in this spectral range make this choice even more attractive. In this spectral region it also works well to use fluorescent dyes including but not limited to AlexaFluor®647 and AlexaFluor®790 for their absorption properties only since the emission wavelengths do not interfere with the coding and detection regions.

Another use is quantum dots as coding labels and terbium or europium chelates for detection with either one of the quantum dots as a reference or another organic UV excitable dye as reference. In this case the AlexaFluor®405, for example, can also function in the detector role. Qdot®545 can be used in conjunction with terbium chelate and Qdot®625 can be used in conjunction with europium chelate. Many combinations are useful with a single violet excitation source.

Still another example of reference beads that can be formulated for any of the systems described previously uses Ruthenium ligand complexes for the reference and a common fluorophore for the reporter such as PacificBlue® or PacificOrange® or Qdots® with violet excitation and or fluorescein/AlexaFluor®488, PE/PE conjugates or PerCP/PerCP conjugates with violet or blue excitation. The ruthenium ligand reference of this embodiment of the present disclosure has relatively broad band emission and is well excited by both violet and blue lasers. It can therefore be monitored in the same channels as many common fluorophores. Using different colored analyte reporters simultaneously is also possible and permits either simultaneously monitoring more than one analyte on one bead or increasing the size of the multiplex array by using differently tagged reporters for different analytes. A 20 analyte array can be made for example using a single coding color (e.g. Qdot®800) of 10 different intensities if 2 reporters are used (e.g. PacificBlue® and PE). The single color array can be expanded to 40 elements if for example 2 colors of reporters are monitored from each laser.

As DNA content in cells in resting phase can be very consistent, antigenic markers on cells can also be quantified relative to a fluorescent DNA stain by using pulsed excitation and measuring the overlap in signal over time with long-lifetime probes used to stain the antigenic markers. Effects not related to excitation that might cause variation in the DNA stain fluorescence relative to fluorescence of the antigenic probes should be minimized. These include temperature, pH and dye loading effects.

Referencing to a DNA stain can similarly be done with long-lifetime DNA probes and short lifetime antigenic probes given an appropriate DNA stain.

Lifetime coding can also be combined with lifetime reference if the emission colors or the excitation of the long-lifetime elements can be well separated. If for example, terbium chelate and a short lifetime UV excited dye are used for coding and ruthenium is used for reference, UV excitation light can be used for coding while violet and or blue light is used for reference and analyte detection.

One embodiment of the present disclosure comprises a method for quantifying an amount of analyte bound to a particle in an acoustic particle analyzer. This method preferably includes manufacturing a particle having a known amount of calibration dye with a long lifetime and a specificity for an analyte. The analyte is bound to the particles and passes the particle through an interrogation zone with a pulsed or modulated laser. The short-lifetime fluorescent signal which relates to the binding event in the interrogation zone is measured and the overlapping fluorescent signal is measured from the long lifetime reference probe. The amount of analyte is then preferably calculated by comparing the analyte related signal to the signal from the known amount of reference label. The analyte related signal is preferably generated by binding a fluorescent ligand specific for the analyte to the analyte such that the particle and analyte and fluorescent ligand form a complex.

Another embodiment of the present disclosure comprises a method for quantifying the amount of analyte bound to a particle in an acoustic particle analyzer. In this embodiment, a particle is manufactured having a known amount of calibration dye with a short lifetime and a specificity for an analyte. This analyte is bound to the particle and passes the particle through an interrogation zone with a pulsed or modulated laser. A long lifetime fluorescent signal that is related to the binding event in the interrogation zone is measured and the overlapping fluorescent signal from the short lifetime reference probe is also measured. The amount of analyte is then calculated by comparing the analyte related signal to the signal from the known amount of reference label. The analyte related signal of this embodiment is preferably generated by binding a fluorescent ligand specific for the analyte to the analyte such that the particle and analyte and fluorescent ligand form a complex.

### ENVIRONMENTAL AND INDUSTRIAL APPLICATIONS

In one embodiment of the present disclosure, acoustic concentration and washing can be used for sample treatment and analysis in a range of environmental and industrial samples, particularly where particles of interest are rare and require significant concentration to acquire a statistically meaningful population. The ability of acoustic concentrators to function as “filterless filters” that are not subject to clogging and periodic replacement requirements makes them very attractive in many applications. Analysis of microbes from municipal water supplies is a prime example. Specific nucleic acid probes and other microbe specific probes are used to confirm the presence of microbes in water samples but pre-concentration before staining is necessary to limit the amount of staining reagent and to process enough volume to be statistically significant. Similar microbial testing is done for a multitude of industrial products and foods from juice, milk and beer to mouthwash and these analyses can also benefit tremendously from acoustic concentration. Acoustic washing can be employed to separate environmental and industrial analytes from reagents such as the staining probes for more sensitive measurements and can also be used to replace the original sample medium with fluids containing different reagents or compositions. Acoustic washing using electrolyte buffer for impedance analysis is of particular utility for virtually any sample including those listed above which does not have the required conductivity for analysis. In an acoustically focused imager, analysis can extend to shape and size of particles which is important for a great deal of industrial processes as diverse as ink production for copiers and printers and quality control in chocolate making. Acoustic focusing and alignment of particles greatly enhances quality of imaging of particles by bringing particles into focus at the focal imaging plane and also orienting asymmetric particles with respect to the acoustic field. Acoustic focusing can be used to concentrate and/or remove particles from waste streams or feed streams. An acoustic focusing apparatus can be placed in-with other filtration systems, e.g. water purification systems, to extend the life of the filters. Such processing is not just limited to aqueous environments, removal of metal, ceramic or other particulates from machining fluids or particulates from spent oils such as motor oils and cooking oils is also possible.

Any of the methods above can be automated with a processor and a database. A computer readable medium containing instructions can preferably cause a program in a data processing medium (a computing system) to perform a method.

The preceding examples can be repeated with similar success by substituting the generically or specifically described components and/or operating conditions of this disclosure for those used in the preceding examples.

Although various embodiments disclosed herein have been described in detail with particular reference to preferred embodiments, other embodiments can achieve the same results. Variations and modifications of the present disclosure will be obvious to those skilled in the art and it is intended to cover in the appended claims all such modifications and equivalents. The entire disclosures of all references, applications, patents, and publications cited above are hereby incorporated by reference.

What is claimed is:

1. A method for analyzing bioparticles comprising:
acoustically focusing one or more bioparticles through an interrogation zone;
optically exciting the one or more bioparticles in the interrogation zone with an excitation source;
detecting an optical signal from the bioparticles; and
analyzing the optical signal to characterize at least one quality or quantity parameter of the bioparticles.

2. The method of claim 1, wherein the bioparticle is a cell, an organelle, a protein, a peptide, or a nucleic acid.

3. The method of claim 1 wherein the bioparticle is labeled.

4. The method of claim 1 wherein the bioparticle is intrinsically fluorescent.

5. The method of claim 1, wherein the type of analysis performed on the bioparticles is one of cell proliferation analysis, live/dead cell discrimination, cell cycle analysis, basic phenotyping, immunophenotyping, rare-event detection, apoptosis, phagocytosis, pinocytosis, detection of phosphoproteins, detection of one or more cellular markers, detection of one or more intracellular marker, detection of cancer cells, detection of pathological markers on a cell, microbial cell analysis or picophytoplankton analysis.

6. The method of claim 5, wherein cell proliferation analysis further includes subjecting the bioparticles to a cell proliferation stimulus prior to the acoustic focusing step.

7. The method of claim 5, wherein immunophenotyping analysis further includes labeling the bioparticles with one or more conjugated antibodies prior to the acoustic focusing step.

8. The method of claim 7, wherein certain optical signals are indicative of a particular immunophenotype.

9. The method of claim 7, wherein the labeled bioparticles are cells which are labeled with multiple conjugated antibodies.

10. The method of claim 7, wherein the cells are blood cells.

11. The method of claim 7, wherein the cells are human blood cells.

12. The method of claim 11, wherein the human blood cells may be immunophenotyped based on the expression of a CD45 marker, a CD3 marker, a CD4 marker, a CD8 marker, a CD19 marker or a CD56 marker.

13. The method of claim 11, wherein the human blood cells may be immunophenotyped as T-cells, B-cells, NK-cells, CD3 T-cells, CD19B-Cells, CD56-NK cells, CD4 T-helper cells, CD8 T-suppressor cells lymphocytes and combinations thereof.

14. The method of claim 9 further comprising performing a multi-color immunophenotyping.

15. A method for detecting phosphoproteins on a cell disposed within a fluid medium, comprising:
stimulating or inhibiting the cell with a kinase or a kinase inhibitor respectively to phorsporylate or de-phosphorylate one or more proteins on the cell;
contacting the cell with one or more antibody specific to detect the one or more phosphorylated protein;
acoustically focusing the cell in the fluid medium;
optically exciting the cell with an excitation source;
detecting an optical signal from the cell; and
analyzing the optical signal, wherein the optical signal is indicative of the presence or absence of the one or more phosphorylated protein.

16. A method for detecting fluorescent protein expression on a cell disposed within a fluid medium, comprising:
transfecting the cell with one or more fluorescent proteins;
acoustically focusing the cell in the fluid medium;
optically exciting the cell with an excitation source;
detecting one or more optical signals from the cell; and
analyzing the optical signal,
wherein the detection of an optical signal corresponding to one or more fluorescent protein is indicative of the presence of expression of the one or more fluorescent proteins and the absence of an optical signal corresponding to one or more fluorescent protein is indicative of the absence of expression of the fluorescent protein.

17. The method of claim 16, wherein the detection of an optical signal corresponding to one or more fluorescent protein is indicative of successful transfection.

18. The method of claim 16, wherein the detection of a first optical signal corresponding to a first fluorescent protein and the detection of a second optical signal corresponding to a second fluorescent protein is indicative of transfection of the cell by the first and the second fluorescent proteins.

19. The method of claim 16, wherein analyzing the optical signal further comprises analyzing the percentage of cells transfected with the one or more fluorescent proteins.

20. The method of claim 16, wherein the fluorescent protein is a red fluorescent protein, a green fluorescent protein, a blue fluorescent protein, a yellow fluorescent protein.

21. A method for detection a rare event within a population of cells, the method comprising:
acoustically focusing the population of cells;
optically exciting the population of cells with an excitation source;
detecting one or more optical signals from the population of cells; and
analyzing the optical signal,
wherein the detection of an optical signal corresponding to a rare event is indicative of the presence of the rare event and the absence of an optical signal corresponding to a rare event is indicative of the absence of the rare event.

22. The method of claim 21, wherein the rare event is the detection of a rare subset of cells within the population of cells.

23. The method of claim 22, wherein the rare subset of cells comprises less than 5% the population of cells.

24. The method of claim 22, further comprising identification of the rare subset of cells.

25. The method of claim 22, comprising the detection of plasmocytoid dendritic cells.

26. The method of claim 22, comprising the detection of CD34+ cells from a population of peripheral blood cells.

27. The method of claim 22, comprising detecting human mesenchymal cells, angiogenic cells, circulating endothelial cells or circulating hematopoietic progenitor cells in human blood.

28. The method of claim 5, wherein different optical signals correspond to different cell cycle phases.

29. The method of claim 28, further comprising quantifying the percentage of cells in one or more cell cycle phases.

30. The method of claim 5, wherein different optical signals correspond to different types of microbial events.

31. The method of claim 30, wherein the microbes are intrinsically fluorescent.

32. The method of claim 30, wherein detection of one or more optical signals are indicative of microbial cell events selected from the group consisting of microbial viability, number of microbial cells, detection of gram positive status of a microbe, detection of gram negative status of a microbe, microbial membrane potential, microbial metabolism and combinations thereof.

33. The method of claim 32, wherein microbial viability comprises detecting live microbial cells separately from dead microbial cells.

34. A method for detecting cell apoptosis, the method comprising:
acoustically focusing one or more cells disposed within a fluid;
optically exciting the one or more cells with an excitation source;
detecting one or more optical signals from the cells; and
analyzing the detected optical signals to identify morphological or biochemical changes that are indicative of cell apoptosis.

35. The method of claim 34, wherein an optical signal corresponding to detecting an apoptotic event in the cell is indicative of an apoptotic cell and the absence of an optical signal corresponding to detecting an apoptotic event in the cell is indicative of the absence of apoptosis.

36. The method of claim 34, wherein the optical signal corresponding to detecting an apoptotic event comprises detecting a change in the cells mitochondrial membrane potential, a change in the cells mitochondrial redox potential, a change in the protein composition in the cells plasma membrane and combinations thereof.

37. The method of claim 34, wherein an optical signal corresponding to detecting translocation of phosphatidylserine (PS) from the inner leaflet of the plasma membrane of the cell to the outermembrane of the plasma membrane of the cell is indicative of an apoptotic cell.
